(19) United States
(12) Patent Application Publication
(10) Pub. No.: US 2025/0122323 A1
(43) Pub. Date: Apr. 17, 2025

(54) ONIUM SALT, BLOCKING AGENT DISSOCIATION CATALYST FOR BLOCKED ISOCYANATE, BLOCKED ISOCYANTE COMPOSITION INCLUDING BLOCKING AGENT DISSOCIATION CATALYST, THERMOSETTING RESIN COMPOSITION, AND CURED PRODUCT AND MANUFACTURING METHOD THEREFOR

(71) Applicant: KOEI CHEMICAL COMPANY, LIMITED, Chiba (JP)

(72) Inventors: Kouya SUGAWARA, Chiba (JP); Motoyoshi MIYAGI, Chiba (JP); Mitsuki ONODA, Chiba (JP)

(73) Assignee: KOEI CHEMICAL COMPANY, LIMITED, Chiba (JP)

(21) Appl. No.: 18/291,365

(22) PCT Filed: Jul. 29, 2022
(86) PCT No.: PCT/JP2022/029359

§ 371 Date: Jan. 23, 2024

(30) Foreign Application Priority Data
Jul. 30, 2021 (JP) 2021-125394

(51) Int. Cl. C08G 18/20; C07C 211/63; C07C 217/08; C07C 233/07; C07C 271/12; C07C 271/28; C07C 275/06; C07D 233/58; C08G 18/18; C08G 18/42; C08G 18/80

(52) U.S. Cl. CPC C08G 18/2027; C07C 211/63; C07C 217/08; C07C 233/07; C07C 271/12; C07C 271/28; C07C 275/06; C07D 233/58; C08G 18/1875; C08G 18/1883; C08G 18/2018; C08G 18/42; C08G 18/8087

(57) ABSTRACT

This invention provides a blocking agent dissociation catalyst for blocked isocyanates comprising an onium salt represented by the following formula (1):

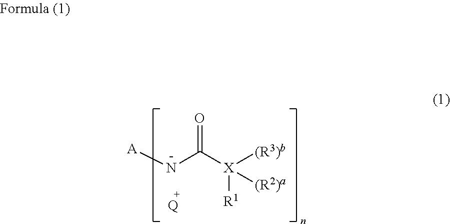

wherein n, R¹, R², R³, A, X, a, b, and Q⁺ are as defined in the specification.

### TECHNICAL FIELD

The present invention relates to an onium salt, a blocking agent dissociation catalyst for blocked isocyanates, a blocked isocyanate composition comprising the blocking agent dissociation catalyst, a thermosetting resin composition, and a cured product and a method for producing the same.

### BACKGROUND ART

Blocked isocyanates are compounds obtained by reaction of an isocyanate with a blocking agent containing active hydrogen groups that are capable of reacting with isocyanate groups. Blocked isocyanates are inactive at ordinary temperatures with highly reactive isocyanate groups being blocked by a blocking agent, and heating causes dissociation of the blocking agent to regenerate the isocyanate groups. Blocked isocyanates having such properties are more excellent in storage stability and easier to handle than isocyanates. Taking advantage of this feature, for example, they are widely used as raw materials for one-component polyurethane resins, which are obtained by curing polyol and isocyanate components, in applications such as paints and adhesives.

As described above, blocked isocyanates require heating to dissociate blocking agents; however, in recent years, there has been a demand for a lower dissociation temperature of blocking agents to reduce energy consumption and costs. Therefore, attempts have been made to use catalysts to lower the dissociation temperature of blocking agents of blocked isocyanates. Organotin compounds, such as dibutyltin dilaurate, are often used as such blocking agent dissociation catalysts; however, due to toxicity issues, the use of these compounds is not preferable. Further, quaternary ammonium methyl carbonate is known as a non-metal blocking agent dissociation catalyst (PTL 1). However, when the inventors used trimethyl-n-hexylammonium methyl carbonate, which is quaternary ammonium methyl carbonate, as a blocking agent dissociation catalyst to react a blocked isocyanate and a polyol, low-temperature curing properties were not satisfactory (see Comparative Example 1).

### CITATION LIST

### Patent Literature

PTL 1: JP2016-222891A

### SUMMARY OF INVENTION

### Technical Problem

The present invention was made in light of the above background art. An object of the present invention is to provide a catalyst that achieves excellent low-temperature dissociation of a blocking agent of a blocked isocyanate. Another object is to provide a blocked isocyanate composition comprising the blocking agent dissociation catalyst, a thermosetting resin composition comprising the blocked isocyanate composition and having excellent low-temperature curing properties, a cured product, and a method for producing the cured product.

### Solution to Problem

The present inventors conducted extensive research to solve the above problem, and found that excellent low-temperature dissociation was achieved when using an onium salt represented by formula (1) as a blocking agent dissociation catalyst for blocked isocyanates. The present invention has thus been completed.

The present invention provides the following blocking agent dissociation catalyst for blocked isocyanates, blocked isocyanate composition, thermosetting resin composition, cured product and method for producing the same, and onium salt.

[1]

A blocking agent dissociation catalyst for blocked isocyanates comprising an onium salt represented by the following formula (1):

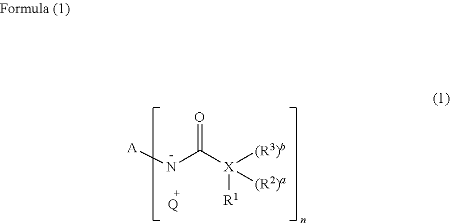

wherein
n represents an integer of 1 or more,
R¹, R², and R³ are the same or different, and each represents a hydrogen atom or a substituted or unsubstituted hydrocarbon group,
A represents a hydrogen atom or a substituted or unsubstituted hydrocarbon group when n=1, and represents a substituted or unsubstituted n-valent hydrocarbon group when n represents an integer of 2 or more,
   wherein some or all of R¹, R², and R³ may be bonded together to form one or more ring structures,
X represents a carbon atom, a nitrogen atom, or an oxygen atom,
a represents 0 or 1,
b represents 0 or 1,
   wherein when X represents a carbon atom, a=1 and b=1, when X represents a nitrogen atom, a=1 and b=0, and when X represents an oxygen atom, a=0 and b=0, and
Q⁺ represents a cation.
   [2]

The blocking agent dissociation catalyst for blocked isocyanates according to [1], wherein in formula (1), Q⁺ represents an organic cation.

[3]

The blocking agent dissociation catalyst for blocked isocyanates according to [1] or [2],
wherein
in formula (1), Q⁺ represents an organic cation represented by the following formula (2) or formula (3):

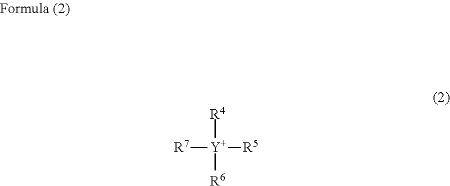

wherein
R⁴, R⁵, R⁶, and R⁷ are the same or different, and each represents a substituted or unsubstituted hydrocarbon group, wherein some or all of R⁴, R⁵, R⁴, and R⁷ may be bonded together to form one or more ring structures, and
Y⁺ represents a nitrogen cation or a phosphorus cation;

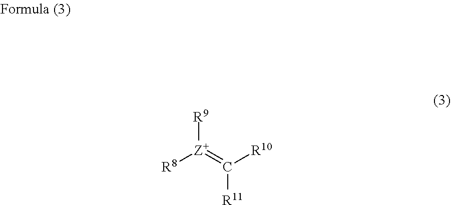

wherein
R⁸, R¹⁰, and R¹¹ are the same or different, and each represents a substituted or unsubstituted hydrocarbon group,
R⁹ represents a substituted or unsubstituted hydrocarbon group,
wherein some or all of R⁸, R⁹, R¹⁰, and R¹¹ may be bonded together to form one or more ring structures, and
Z⁺ represents a nitrogen cation or a phosphorus cation.
   [4]

The blocking agent dissociation catalyst for blocked isocyanates according to [3],
wherein
Y⁺ represents a nitrogen cation, or the organic cation represented by formula (3) represents an organic cation represented by formula (3-1) or formula (3-2):

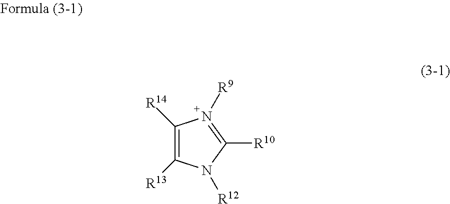

wherein
R⁹ and R¹² each represent a substituted or unsubstituted hydrocarbon group, and
R¹⁰, R¹³, and R¹⁴ are the same or different, and each represents a hydrogen atom or a substituted or unsubstituted hydrocarbon group, wherein some or all of R⁹, R¹⁰, R¹², R¹³, and R¹⁴ may be bonded together to form one or more ring structures;

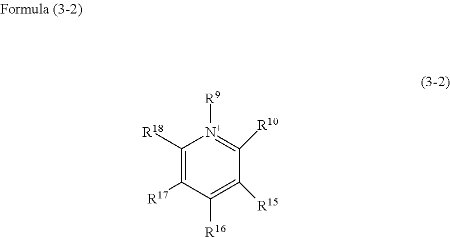

wherein
R⁹ represents a substituted or unsubstituted hydrocarbon group, and
R¹⁰, R¹⁵, R¹⁶, R¹⁷, and R¹⁸ are the same or different, and each represents a hydrogen atom or a substituted or unsubstituted hydrocarbon group, wherein some or all of R⁹, R¹⁰, R¹⁵, R¹⁶, R¹⁷, and R¹⁸ may be bonded together to form one or more ring structures.
   [5]

The blocking agent dissociation catalyst for blocked isocyanates according to [1],
wherein
A represents a hydrogen atom, a substituted or unsubstituted aliphatic hydrocarbon group, a substituted or unsubstituted alicyclic hydrocarbon group, a substituted or unsubstituted aromatic hydrocarbon group, or a substituted or unsubstituted aromatic aliphatic hydrocarbon group.
   [6]

The blocking agent dissociation catalyst for blocked isocyanates according to any one of [1] to [5], wherein n represents an integer of 1 to 20.

[7]

The blocking agent dissociation catalyst for blocked isocyanates according to any one of [1] to [6],
wherein
when X represents a carbon atom, R¹, R², and R³ are the same or different, and each represents a hydrogen atom or a substituted or unsubstituted alkyl group,
when X represents a nitrogen atom, R¹ and R² are the same or different, and each represents a hydrogen atom or a substituted or unsubstituted alkyl group, and
when X represents an oxygen atom, R¹ represents a substituted or unsubstituted alkyl group.
   [8]

A blocked isocyanate composition comprising the blocking agent dissociation catalyst for blocked isocyanates of any one of [1] to [7] and a blocked isocyanate compound.

[9]

The blocked isocyanate composition according to [8], wherein the blocked isocyanate compound is a blocked isocyanate compound blocked with at least one blocking agent selected from the group consisting of alcohol compounds, phenol compounds, amine compounds, lactam compounds, oxime compounds, keto-enol compounds, active methylene compounds, pyrazole compounds, triazole compounds, imide compounds, mercaptan compounds, imine compounds, urea compounds, and diaryl compounds.

[10]

The blocked isocyanate composition according to [8] or [9], wherein the blocked isocyanate compound is a blocked isocyanate compound blocked with a fluorinated alcohol compound.

[11]

A thermosetting resin composition comprising the blocked isocyanate composition of any one of [8] to [10] and a compound having an isocyanate-reactive group.

[12]

The thermosetting resin composition according to [11], wherein the compound having an isocyanate-reactive group is a polyol compound.

[13]

A cured product obtained by curing the thermosetting resin composition of [11] or [12].

[14]

A method for producing a cured product, comprising curing the thermosetting resin composition of [11] or [12] by heating.

[15]

An onium salt represented by the following formula (1):

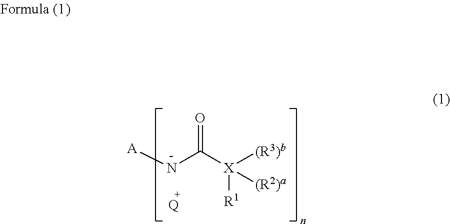

wherein
n represents an integer of 1 or more,
R¹, R², and R³ are the same or different, and each represents a hydrogen atom or a substituted or unsubstituted hydrocarbon group,
A represents a hydrogen atom or a substituted or unsubstituted hydrocarbon group when n=1, and represents a substituted or unsubstituted n-valent hydrocarbon group when n represents an integer of 2 or more,
   wherein some or all of R¹, R², and R³ may be bonded together to form one or more ring structures,
X represents a carbon atom, a nitrogen atom, or an oxygen atom, a represents 0 or 1, b represents 0 or 1,
   wherein when X represents a carbon atom, a=1 and b=1, when X represents a nitrogen atom, a=1 and b=0, and when X represents an oxygen atom, a=0 and b=0, and Q⁺ represents an organic cation represented by the following formula (2) or formula (3):

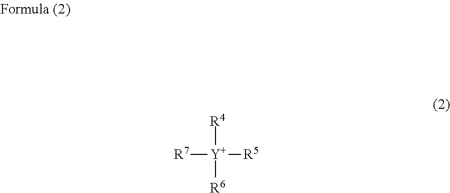

wherein
R⁴, R⁵, R⁶, and R⁷ are the same or different, and each represents a substituted or unsubstituted hydrocarbon group, wherein some or all of R⁴, R⁵, R⁶, and R⁷ may be bonded together to form one or more ring structures, and Y⁺ represents a nitrogen cation or a phosphorus cation;

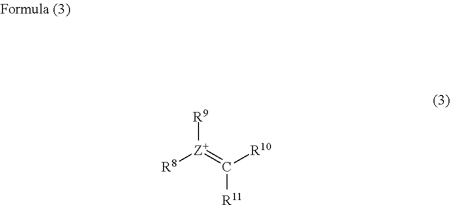

wherein
R⁸, R¹⁰, and R¹¹ are the same or different, and each represents a substituted or unsubstituted hydrocarbon group,
R⁹ represents a substituted or unsubstituted hydrocarbon group,
wherein some or all of R^{A}, R⁹, R¹⁰, and R¹¹ may be bonded together to form one or more ring structures, and
Z⁺ represents a nitrogen cation or a phosphorus cation; with the proviso that 1,3-di-isopropyl-4,5-dimethylimidazolium N,N′-diphenylureate is excluded.
   [16]

The onium salt according to [15],
wherein
Y⁺ represents a nitrogen cation, or the organic cation represented by formula (3) is an organic cation represented by formula (3-1) or formula (3-2):

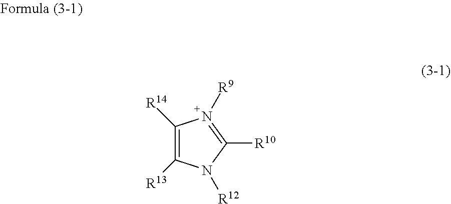

wherein
R⁹ and R¹² each represent a substituted or unsubstituted hydrocarbon group, and
R¹⁰, R¹³, and R¹⁴ are the same or different, and each represents a hydrogen atom or a substituted or unsubstituted hydrocarbon group, wherein some or all of R⁹, R¹⁰, R¹², R¹³, and R¹⁴ may be bonded together to form one or more ring structures;

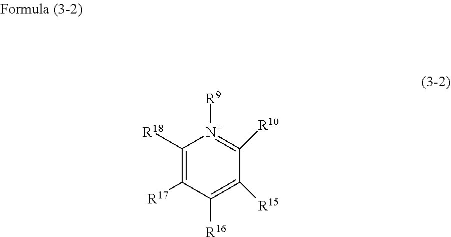

wherein
R⁹ represents a substituted or unsubstituted hydrocarbon group, and
R¹⁰, R¹⁵, R¹⁶, R¹⁷, and R¹⁸ are the same or different, and each represents a hydrogen atom or a substituted or unsubstituted hydrocarbon group, wherein some or all of R⁹, R¹⁰, R¹⁵, R¹⁶, R¹⁷, and R¹⁸ may be bonded together to form one or more ring structures.
   [17]

The onium salt according to [16],
wherein
A represents a substituted or unsubstituted n-valent aliphatic hydrocarbon group, a substituted or unsubstituted n-valent alicyclic hydrocarbon group, a substituted or unsubstituted n-valent aromatic hydrocarbon group, or a substituted or unsubstituted n-valent aromatic aliphatic hydrocarbon group.
   [18]

The onium salt according to any one of [15] to [17], wherein n represents an integer of 1 to 20.

[19]

The onium salt according to [15], containing one anion selected from (4-1) to (4-4) and one organic cation selected from (b-i) and (b-ii):

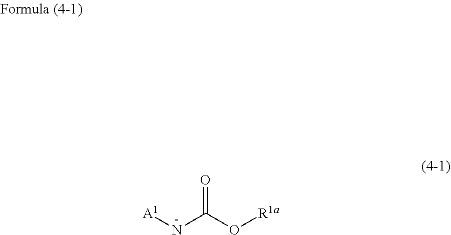

wherein
A¹ represents a substituted or unsubstituted aromatic hydrocarbon group, and
R^{1a} represents a substituted or unsubstituted aliphatic hydrocarbon group;

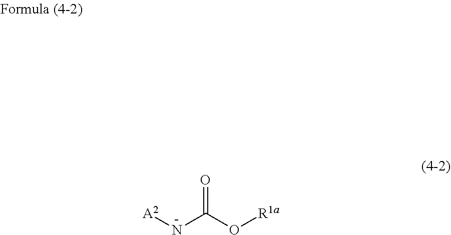

wherein
A² and R^{1a} are the same or different, and each represents a substituted or unsubstituted aliphatic hydrocarbon group;

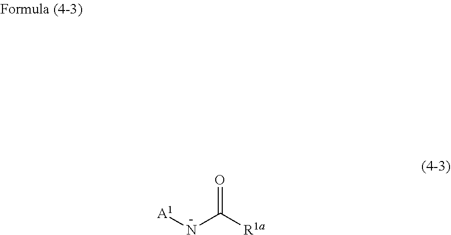

wherein A¹ and R^{1a} are as defined above;

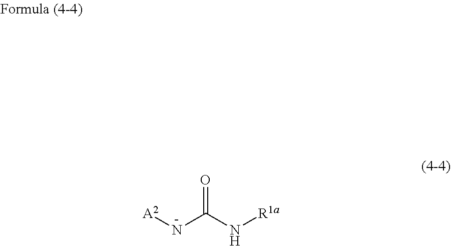

wherein A² and R^{1a} are as defined above;
(b-i) an organic cation represented by formula (2):

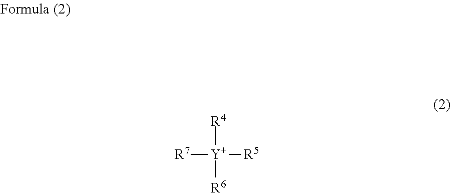

wherein
R⁴, R⁵, R⁶, and R⁷ are the same or different, and each represents a substituted or unsubstituted hydrocarbon group, wherein some or all of R⁴, R⁵, R⁶, and R⁷ may be bonded together to form one or more ring structures, and
Y⁺ represents a nitrogen cation;
(b-ii) an organic cation represented by the following formula (3-1):

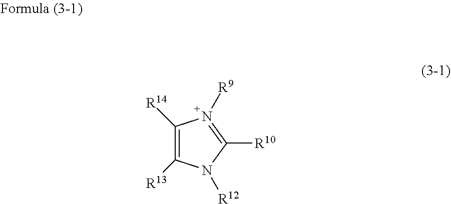

wherein
R⁹ and R¹² each represent a substituted or unsubstituted hydrocarbon group, and R¹⁰, R¹³, and R¹⁴ are the same or different, and each represents a hydrogen atom or a substituted or unsubstituted hydrocarbon group, wherein some or all of R⁹, R¹⁰, R¹², R¹³, and R¹⁴ may be bonded together to form one or more ring structures.
   [20]

The onium salt according to [15], containing one anion selected from (a-i) to (a-vi) and one organic cation selected from (b-i) and (b-ii):

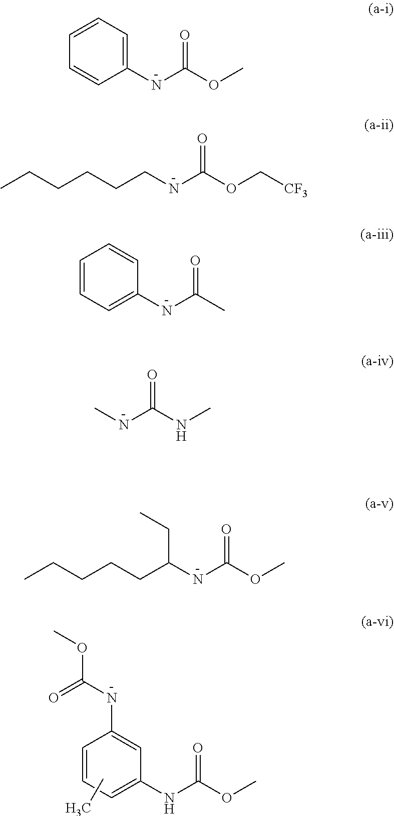

(b-i) an organic cation represented by formula (2):

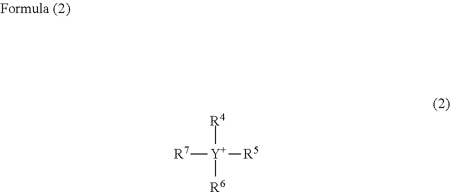

wherein
R⁴, R⁵, R⁶, and R⁷ are the same or different, and each represents a substituted or unsubstituted hydrocarbon group, wherein some or all of R⁴, R⁵, R⁶, and R⁷ may be bonded together to form one or more ring structures, and
Y⁺ represents a nitrogen cat ion;
(b-ii) an organic cation represented by the following formula (3-1):

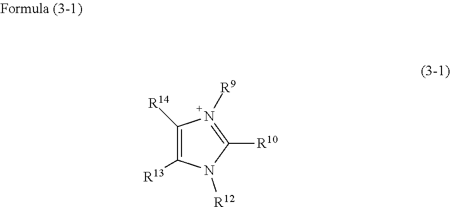

wherein
R⁹ and R¹² each represent a substituted or unsubstituted hydrocarbon group, and
R¹⁰, R¹³, and R¹⁴ are the same or different, and each represents a hydrogen atom or a substituted or unsubstituted hydrocarbon group, wherein some or all of R⁹, R¹⁰, R¹², R¹³, and R¹⁴ may be bonded together to form one or more ring structures.
   [21]

The onium salt according to [15], wherein the onium salt is represented by any one of the following:

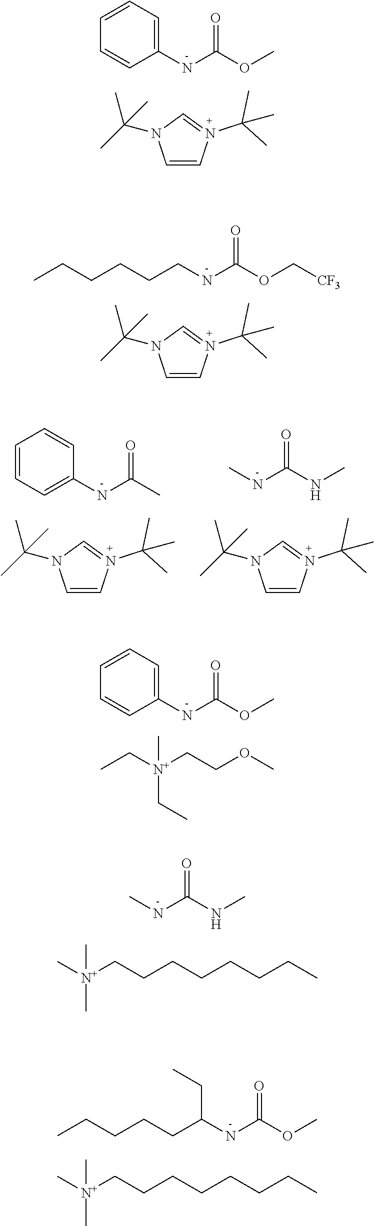

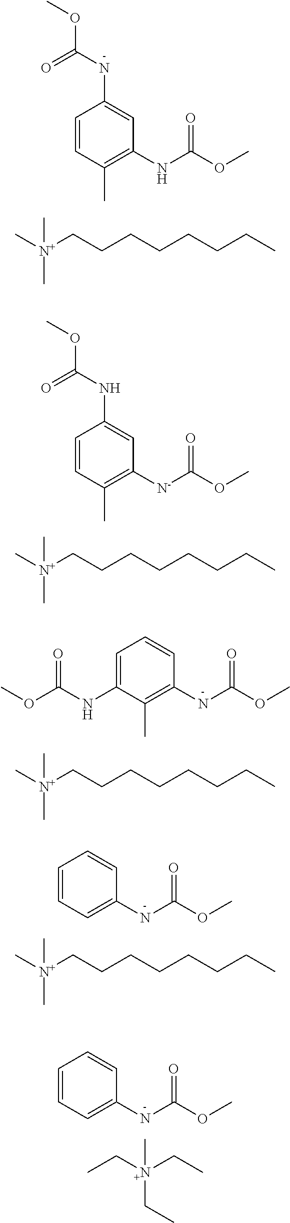

### Advantageous Effects of Invention

Provided is a catalyst that achieves excellent low-temperature dissociation of a blocking agent of a blocked isocyanate. Further provided are a blocked isocyanate composition comprising the blocking agent dissociation catalyst, a thermosetting resin composition comprising the blocked isocyanate composition and having excellent low-temperature curing properties, and a cured product and a method for producing the same.

### BRIEF DESCRIPTION OF DRAWINGS

FIG. 1 shows the results of ¹H-NMR analysis in Production Example 8 of the present application.

FIG. 2 shows the results of IR analysis in Production Example 8 of the present application.

FIG. 3 shows the results of ¹H-NMR analysis in Production Example A-10 of the present application.

FIG. 4 shows the results of IR analysis in Production Example A-10 of the present application.

### DESCRIPTION OF EMBODIMENTS

### Blocking Agent Dissociation Catalyst for Blocked Isocyanates

As a blocking agent dissociation catalyst for blocked isocyanates of the present invention, an onium salt represented by formula (1) (hereafter referred to as “the onium salt (1)”) can be used.

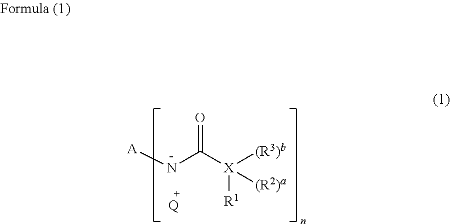

(In the formula, n represents an integer of 1 or more, R¹, R², and R³ are the same or different, and each represents a hydrogen atom or a substituted or unsubstituted hydrocarbon group,
A represents a hydrogen atom or a substituted or unsubstituted hydrocarbon group when n=1, and represents a substituted or unsubstituted n-valent hydrocarbon group when n represents an integer of 2 or more,
   wherein some or all of R¹, R², and R³ may be bonded together to form one or more ring structures,
X represents a carbon atom, a nitrogen atom, or an oxygen atom,
a represents 0 or 1,
b represents 0 or 1,
   wherein when X represents a carbon atom, a=1 and b=1, when X represents a nitrogen atom, a=1 and b=0, and when X represents an oxygen atom, a=0 and b=0, and Q represents a cation.)

In formula (1), the cation represented by Q⁺ may be an organic cation, preferably a nitrogen-containing organic cation or an organic phosphonium cation, more preferably an organic cation represented by the following formula (2) or formula (3), even more preferably an organic cation represented by the following formula (2), formula (3-1), or formula (3-2), and particularly preferably an organic cation represented by the following formula (2) or formula (3-1).

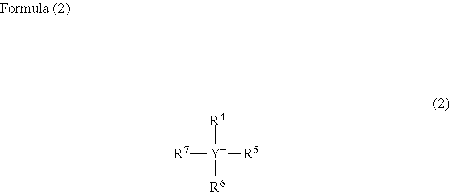

(In the formula,
R⁴, R⁵, R⁶, and R⁷ are the same or different, and each represents a substituted or unsubstituted hydrocarbon group, wherein some or all of R⁴, R⁵, R⁴, and R⁷ may be bonded together to form one or more ring structures, and Y⁺ represents a nitrogen cation or a phosphorus cation)

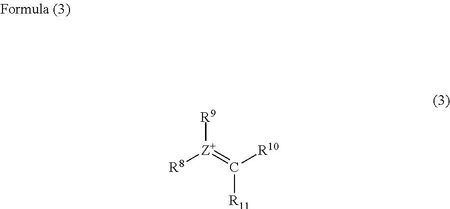

(In the formula,
R⁸, R⁹, and R¹¹ are the same or different, and each represents a substituted or unsubstituted hydrocarbon group,
R¹⁰ represents a hydrogen atom, or a substituted or unsubstituted hydrocarbon group, wherein some or all of R⁸, R⁹, R¹⁰, and R¹¹ may be bonded together to form one or more ring structures, and
Z⁺ represents a nitrogen cation or a phosphorus cation)

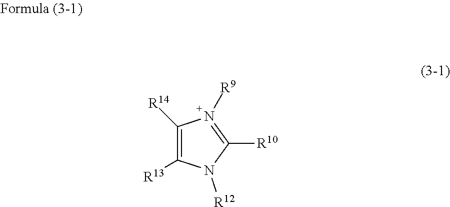

(In the formula,
R⁹ and R¹² each represent a substituted or unsubstituted hydrocarbon group, and
R¹⁰, R¹³, and R¹⁴ are the same or different, and each represents a hydrogen atom or a substituted or unsubstituted hydrocarbon group, wherein some or all of R⁹, R¹⁰, R¹², R¹³, and R¹⁴ may be bonded together to form one or more ring structures)

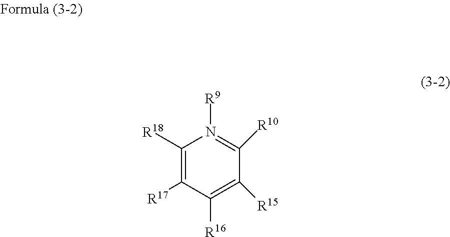

(In the formula,
R⁹ represents a substituted or unsubstituted hydrocarbon group, and
R¹⁰, R¹⁵, R¹⁶, R¹⁷, and R¹⁸ are the same or different, and each represents a hydrogen atom or a substituted or unsubstituted hydrocarbon group, wherein some or all of R⁹, R¹⁰, R¹⁵, R¹⁶, R¹⁷, and R¹⁸ may be bonded together to form one or more ring structures.)

In the onium salt (1), the anion is preferably an anion represented by formula (4), more preferably an organic anion represented by any one of the following formulas (4-1) to (4-4), and particularly preferably any one of the following formulas (a-i) to (a-vi).

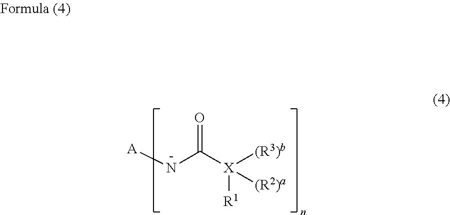

(In the formula, n, R¹, R², R³, A, a, b, and X are as defined above)

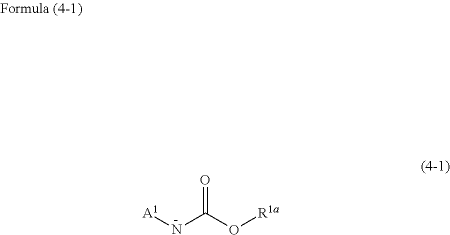

(In the formula,
A¹ represents a substituted or unsubstituted aromatic hydrocarbon group, and
R^{1a} represents a substituted or unsubstituted aliphatic hydrocarbon group)

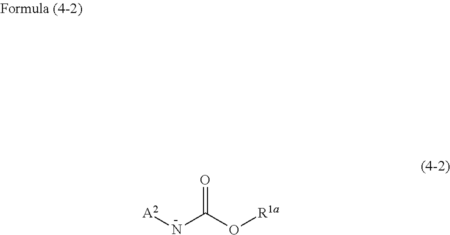

(In the formula, A² and R^{1a} are the same or different, and each represents a substituted or unsubstituted aliphatic hydrocarbon group)

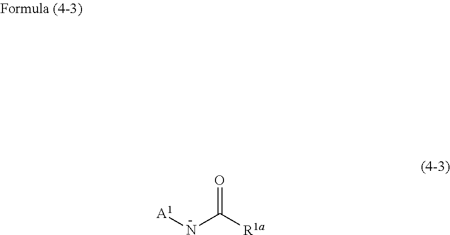

(In the formula, A¹ and R^{1a} are as defined above)

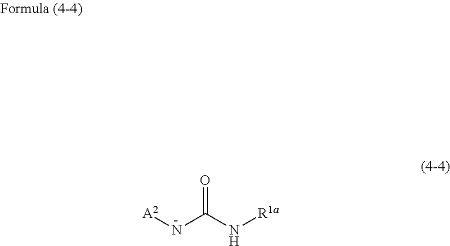

(In the formula, A² and R^{1a} are as defined above)

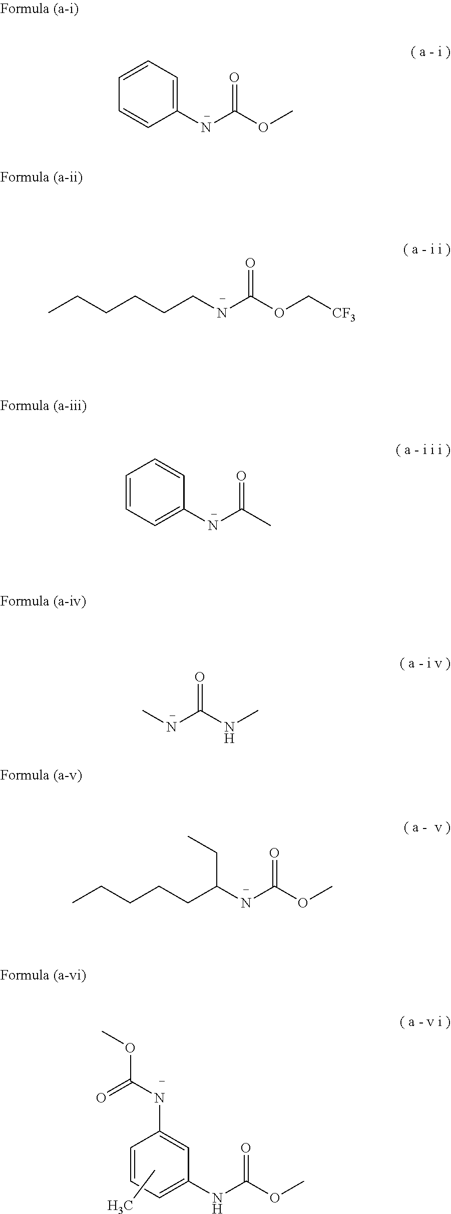

In one embodiment, in formula (1) and formula (4), R¹, R², and R³ each represent a hydrogen atom or a substituted or unsubstituted hydrocarbon group, preferably a substituted or unsubstituted C₁-C₁₀₀ hydrocarbon group, more preferably a substituted or unsubstituted C₁-C₅₀ hydrocarbon group, and particularly preferably a substituted or unsubstituted C₁-C₃₀ hydrocarbon group.

In another embodiment, in formula (1) and formula (4), R¹, R², and R³ each represent a hydrogen atom or a substituted or unsubstituted alkyl group, preferably a substituted or unsubstituted C₁-C₁₀₀ alkyl group, more preferably a substituted or unsubstituted C₁-C₅₀ alkyl group, and particularly preferably a substituted or unsubstituted C₁-C₃₀ alkyl group.

In another embodiment, in formula (1) and formula (4), R¹, R², and R³ each represent a substituted or unsubstituted aliphatic hydrocarbon group, alicyclic hydrocarbon group, aromatic hydrocarbon group, or aromatic aliphatic hydrocarbon group, preferably a substituted or unsubstituted C₁-C₁₀₀ aliphatic hydrocarbon group, alicyclic hydrocarbon group, aromatic hydrocarbon group, or aromatic aliphatic hydrocarbon group, more preferably a substituted or unsubstituted C₁-C₅₀ (aliphatic hydrocarbon group, alicyclic hydrocarbon group, aromatic hydrocarbon group, or aromatic aliphatic hydrocarbon group, and particularly preferably a substituted or unsubstituted C₁-C₃₀ aliphatic hydrocarbon group, alicyclic hydrocarbon group, aromatic hydrocarbon group, or aromatic aliphatic hydrocarbon group.

In one embodiment, in formula (1) and formula (4), A represents a hydrogen atom or a substituted or unsubstituted n-valent hydrocarbon group, and preferably a hydrogen atom, a substituted or unsubstituted n-valent C₁-C₃₀ aliphatic hydrocarbon group, a substituted or unsubstituted n-valent C₃-C₃₀ alicyclic hydrocarbon group, a substituted or unsubstituted n-valent C₅-C₂₀₀ aromatic hydrocarbon group, or a substituted or unsubstituted n-valent C₇-C₂₀₀ aromatic aliphatic hydrocarbon group.

The “n-valent hydrocarbon group” refers to a group obtained by removing n hydrogens from a hydrocarbon group. The “n-valent aliphatic hydrocarbon group” refers to a group obtained by removing n hydrogens from an aliphatic hydrocarbon group. The “n-valent alicyclic hydrocarbon group” refers to a group obtained by removing n hydrogens from an alicyclic hydrocarbon group. The “n-valent aromatic hydrocarbon group” refers to a group obtained by removing n hydrogens from an aromatic hydrocarbon group. The “n-valent aromatic aliphatic hydrocarbon group” refers to a group obtained by removing n hydrogens from an aromatic aliphatic hydrocarbon group.

In the present specification, the “substituted or unsubstituted (n-valent) hydrocarbon groups” include (i) an (n-valent) hydrocarbon group that may have a substituent, (ii) an (n-valent) hydrocarbon group that may be substituted with a heteroatom, and (iii) a hydrocarbon group having a substituent and substituted with a heteroatom.

Further, the “substituted or unsubstituted aliphatic hydrocarbon groups” include (iv) an (n-valent) aliphatic hydrocarbon group that may have a substituent, (v) an (n-valent) aliphatic hydrocarbon group that may be substituted with a heteroatom, and (vi) an (n-valent) aliphatic hydrocarbon group having a substituent and substituted with a heteroatom.

Further, the “substituted or unsubstituted (n-valent) alicyclic hydrocarbon groups” include (vii) an (n-valent) alicyclic hydrocarbon group that may have a substituent, (viii) an (n-valent) alicyclic hydrocarbon group that may be substituted with a heteroatom, and (ix) an (n-valent) alicyclic hydrocarbon group having a substituent and substituted with a heteroatom.

Further, the “substituted or unsubstituted (n-valent) aromatic hydrocarbon groups” include (x) an (n-valent) aromatic hydrocarbon group that may have a substituent, (xi) an (n-valent) aromatic hydrocarbon group that may be substituted with a heteroatom, and (xii) an (n-valent) aromatic hydrocarbon group having a substituent and substituted with a heteroatom.

Further, the “substituted or unsubstituted (n-valent) aromatic aliphatic hydrocarbon groups” include (xiii) an (n-valent) aromatic aliphatic hydrocarbon group that may have a substituent, (xiv) an (n-valent) aromatic aliphatic hydrocarbon group that may be substituted with a heteroatom, and (xv) an (n-valent) aromatic aliphatic hydrocarbon group having a substituent and substituted with a heteroatom.

The unsubstituted hydrocarbon group is, for example, a methyl, ethyl, propyl, isopropyl, butyl, sec-butyl, tert-butyl, pentyl, hexyl, heptyl, decyl, dodecyl, octadecyl, cyclopropyl, cyclopentyl, cyclohexyl, phenyl, naphthyl, benzyl, phenethyl, tolyl, or allyl group.

The unsubstituted aliphatic hydrocarbon group is, for example, a methyl, ethyl, propyl, isopropyl, butyl, sec-butyl, tert-butyl, pentyl, hexyl, heptyl, decyl, dodecyl, octadecyl, or allyl group.

The unsubstituted alicyclic hydrocarbon group is, for example, a cyclopropyl, cyclopentyl, or cyclohexyl group.

The unsubstituted aromatic hydrocarbon group is, for example, a phenyl or tolyl group.

The unsubstituted aromatic aliphatic hydrocarbon group is, for example, a benzyl, phenethyl, or xylylene group.

In the present specification, examples of substituents include halogen atoms, such as fluorine, chlorine, bromine, and iodine; alkylamino groups, such as methylamino; dialkylamino groups, such as dimethylamino; alkoxy groups, such as methoxy and ethoxy; aryloxy groups, such as phenoxy and naphthyloxy; aralkyloxy groups, such as benzyloxy and naphthylmethoxy; halogenated alkyl groups, such as trifluoromethyl; a nitro group, a cyano group, a sulfonyl group, alkylcarbonylamino groups, alkyloxycarbonylamino groups, (alkylamino) carbonylamino groups, (dialkylamino)carbonylamino groups, and the like. Each hydrocarbon group of R¹, R², and R³ may be substituted with at least one heteroatom, such as oxygen, nitrogen, or sulfur. When each hydrocarbon group of R¹, R², and R³ is substituted with at least one heteroatom, such as oxygen, nitrogen, or sulfur, the hydrocarbon group has at least one group, such as —O—, —N<, —NH—, —S—, or —SO₂—, and the hydrocarbon chain is interrupted by such a group.

Examples of the alkyl moiety of the above alkylamino groups, dialkylamino groups, alkoxy groups, halogenated alkyl groups, alkylcarbonylamino groups, alkyloxycarbonylamino groups, (alkylamino) carbonylamino groups, and (dialkylamino) carbonylamino groups include linear or branched C₁-C₁₂ alkyl groups, such as methyl, ethyl, propyl, isopropyl, n-butyl, isobutyl, sec-butyl, tert-butyl, pentyl, 1-ethylpentyl, heptyl, octyl, and 2-ethylhexyl. The number of carbon atoms in the alkyl group is preferably 1 to 8, and more preferably 1 or 2.

Examples of the aryl moiety of the above aryloxy groups include C₆-C₁₀ aryl groups. Specific examples of the aryl moiety include a phenyl group, a naphthyl group, and the like.

Examples of the aralkyl moiety of the above aralkyloxy groups include C₇-C₁₂ aralkyl groups. Specific examples of the aralkyl moiety include a benzyl group, a naphthylmethyl group, and the like.

In A, examples of the unsubstituted n-valent hydrocarbon group include groups obtained by removing n hydrogen atoms from methane, ethane, propane, isopropane, butane, sec-butane, tert-butane, pentane, hexane, heptane, 2-ethylhexane, decane, dodecane, octadecane, cyclopropane, cyclopentane, cyclohexane, benzene, naphthalene, toluene, phenylethane, and propylene. In terms of toluene and phenylethane, either one or more hydrogen atoms of the aromatic ring or one or more hydrogen atoms of the methyl group or ethyl group, or both, may have been removed. However, when n represents 2 or more, hydrogen atoms for removal are those bonding to different carbon atoms.

In another embodiment, A represents a substituted or unsubstituted branched-chain hydrocarbon group, preferably a substituted or unsubstituted C₁-C₁₀₀ chain hydrocarbon group, more preferably a substituted or unsubstituted C₁-C₅₀ chain hydrocarbon group, and particularly preferably a substituted or unsubstituted C₁-C₃₀ chain hydrocarbon group.

Examples of the “chain hydrocarbon group” include branched alkyl groups, branched alkenyl groups, branched alkynyl groups, and the like.

Specific examples of the “chain hydrocarbon group” include an isopropyl group, a 2-ethylhexyl group, and the like.

When R⁴, R⁵, R⁶, and R⁷ described below each represent an unsubstituted hydrocarbon group, in particular, an unsubstituted alkyl group, cycloalkyl group, aryl group, or aralkyl group, and when n=1, in one embodiment, A preferably represents a substituted or unsubstituted branched-chain hydrocarbon group.

In yet another embodiment, in formula (1) and formula (4), A represents a hydrocarbon group, excluding the following isocyanate groups of isocyanates of (i) to (v). In the present specification, isocyanates include monofunctional and polyfunctional isocyanates.
(i) Aliphatic isocyanates
(ii) Alicyclic isocyanates
(iii) Aromatic isocyanates
(iv) Aromatic aliphatic isocyanates
(v) Modified isocyanates formed from at least one member selected from the group consisting of aliphatic isocyanates, alicyclic isocyanates, aromatic isocyanates, and aromatic aliphatic isocyanates

In the present specification, specific examples of preferred groups represented by A are shown below.

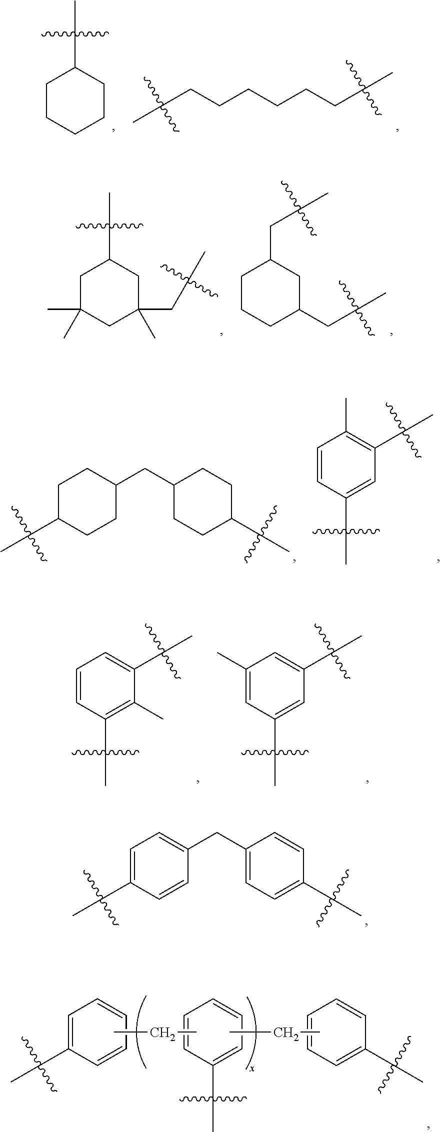

x represents an integer of 0 or more and 20 or less, and preferably an integer of 1 or more and 20 or less.

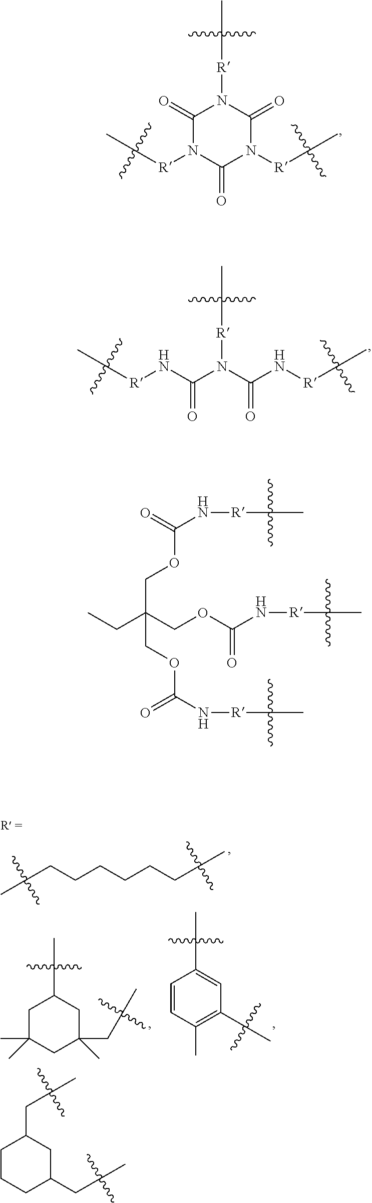

In formula (1) and formula (4), X represents a carbon atom, a nitrogen atom, or an oxygen atom, and preferably a nitrogen atom or an oxygen atom.

In formula (1) and formula (4), n represents an integer of 1 or more, preferably an integer of 1 to 20, more preferably 1 to 6, even more preferably 1 to 4, and particularly preferably 1 or 2.

In formula (4-1), formula (4-2), formula (4-3), and formula (4-4), A=represents a substituted or unsubstituted aromatic hydrocarbon group, preferably a substituted or unsubstituted C₁-C₁₀₀ aromatic hydrocarbon group, more preferably a substituted or unsubstituted C₁-C₁₀₀ aromatic hydrocarbon group, and particularly preferably a substituted or unsubstituted C₁-C₃₀ aromatic hydrocarbon group.

In formula (4-1), formula (4-2), formula (4-3), and formula (4-4), A² and R^{1a} each represent a substituted or unsubstituted aliphatic hydrocarbon group, preferably a substituted or unsubstituted C₁-C₁₀₀ aliphatic hydrocarbon group, more preferably a substituted or unsubstituted C₁-C₅₀ aliphatic hydrocarbon group, and particularly preferably a substituted or unsubstituted C₁-C₃₀ aliphatic hydrocarbon group.

In formula (2), R⁴, R⁵, R⁶, and R⁷ each represent a substituted or unsubstituted hydrocarbon group, preferably a substituted or unsubstituted C₁-C₁₀₀ hydrocarbon group, more preferably a substituted or unsubstituted C₁-C₅₀ hydrocarbon group, and particularly preferably a substituted or unsubstituted C₁-C₃₀ hydrocarbon group.

In another embodiment, R⁴, R⁵, R⁶ and R⁷ each represent a substituted or unsubstituted aliphatic hydrocarbon group, alicyclic hydrocarbon group, aromatic hydrocarbon group, or aromatic aliphatic hydrocarbon group, preferably a substituted or unsubstituted C₁-C₁₀₀ aliphatic hydrocarbon group, alicyclic hydrocarbon group, aromatic hydrocarbon group, or aromatic aliphatic hydrocarbon group, more preferably a substituted or unsubstituted C₁-C₅₀ aliphatic hydrocarbon group, alicyclic hydrocarbon group, aromatic hydrocarbon group, or aromatic aliphatic hydrocarbon group, and particularly preferably a substituted or unsubstituted C₁-C₃₀ aliphatic hydrocarbon group, alicyclic hydrocarbon group, aromatic hydrocarbon group, or aromatic aliphatic hydrocarbon group.

In formula (2), Y⁺ represents a nitrogen cation (N⁺) or a phosphorus cation (P⁺), and preferably a nitrogen cation.

In formula (2), when Y⁺ represents a nitrogen cation, at least one of R⁴, R⁵, R⁶, and R⁷ is preferably a hydrocarbon group having one or more substituents independently selected from the group consisting of halogen atoms, halogenated alkyl groups, alkoxy groups, aryloxy groups, alkyloxycarbonyl groups, aryloxycarbonyl groups, alkyloxycarbonylamino groups, aryloxycarbonylamino groups, monoalkylsiloxy groups, dialkylsiloxy groups, trialkylsiloxy groups, monoarylsiloxy groups, diarylsiloxy groups, triarylsiloxy groups, monoalkylsilyl groups, dialkylsilyl, trialkylsilyl groups, hydroxy, nitro, and cyano groups, and particularly preferably a hydrocarbon group having an alkoxy group.

Examples of the organic cation represented by formula (2) include tetramethylanonium, tetraethylammonium, tetrapropylammonium, tetrabutylannonium, tetrapentylamnonium, tetrahexylammonium, tetraheptylammonium, tetraoctylammonium, tetranonylammonium, tetra(decyl)ammonium, N-ethyl-N,N,N-trimethylammonium, N,N,N-trimethyl-N-propylamnonium, N-butyl-N,N,N-trimethylanonium, N-octyl-N,N,N-trimethylannonium, N,N,N-triethyl-N-methylamnonium, N,N,N-triethyl-N-decylammonium, N,N,N-triethyl-N-eicosylamnonium, N,N,N-tributyl-N-pentylammonium, N,N,N-tributyl-N-hexylammonium, N,N,N-tributyl-N-heptylammonium, N,N,N-tributyl-N-octylamnonium, N,N,N-tributyl-N-nonylammonium, N,N,N-tributyl-N-decylammonium, N,N,N-tributyl-N-eicosylammonium, N,N-diethyl-N-methyl-N-propylannonium, N-butyl-N,N-diethyl-N-methylannonium, N,N-dimethylpyrrolidinium, N,N-dimethylpiperidinium, N,N,N-trimethyl-N-(2-methoxyethyl)amnonium, N,N-diethyl-N-(2-methoxyethyl)-N-methylamnonium, N, N,N-triethyl-N-(2-ethoxyethyl)ammonium, N,N-diethyl-N-propyl-N-(2-ethoxyethyl)ammonium, N-ethyl-N-[2-(2-methoxyethoxy)ethyl]-N,N-dimethylannonium, N,N-diethyl-N-[2-(2-methoxyethoxy)ethyl]-N-methylammonium, N,N-di(2-methoxyethyl)-N,N-dimethylammonium, N,N-di(2-ethoxyethyl)-N,N-dimethylammonium, N-[2-(2-methoxyethoxy)ethyl]-N-(2-methoxyethyl)-N,N-dimethylaumonium, N-(2-ethoxyethyl)-N-[2-(2-methoxyethoxy)ethyl]-N, N-dimethylammonium, tetramethylphosphonium, tetraethylphosphonium, tetrapropylphosphonium, tetrabutylphosphonium, tetrapentylphosphonium, tetrahexylphosphonium, tetraheptylphosphonium, tetraoctylphosphonium, tetranonylphosphonium, tetra(decyl)phosphonium, tributyloctylphosphonium, tri-n-butyl-n-hexylphosphonium, tributyldodecylphosphonium, trihexyltetradecylphosphonium, trimethyl (2-methoxyethyl)phosphonium, diethyl (2-methoxyethyl)methylphosphonium, triethyl (2-ethoxyethyl)phosphonium, diethylpropyl (2-ethoxyethyl)phosphonium, ethyl[2-(2-methoxyethoxy)ethyl]dimethylphosphonium, diethyl[2-(2-methoxyethoxy)ethyl]methylphosphonium, di(2-methoxyethyl)dimethylphosphonium, di(2-ethoxyethyl)dimethylphosphonium, P-[2-(2-methoxyethoxy)ethyl](2-methoxyethyl)dimethylphosphonium, (2-ethoxyethyl) [2-(2-methoxyethoxy)ethyl]dimethylphosphonium, and the like.

Preferred are N,N,N-trimethyl-N-(2-methoxyethyl)ammonium, N,N-diethyl-N-(2-methoxyethyl)-N-methylammonium, N,N,N-triethyl-N-(2-ethoxyethyl)ammonium, N,N-diethyl-N-propyl-N-(2-ethoxyethyl)ammonium, N-ethyl-N-[2-(2-methoxyethoxy)ethyl]-N,N-dimethylammonium, N,N-diethyl-N-[2-(2-methoxyethoxy)ethyl]-N-methylammonium, N,N-di(2-methoxyethyl)-N,N-dimethylammonium, N,N-di(2-ethoxyethyl)-N,N-dimethylammonium, N-[2-(2-methoxyethoxy)ethyl]-N-(2-methoxyethyl)-N, N-dimethylamonium, N-(2-ethoxyethyl)-N-[2-(2-methoxyethoxy)ethyl]-N,N-dimethylammonium, and tri-n-butyl-n-hexylphosphonium, and particularly preferred are N,N-diethyl-N-(2-methoxyethyl)-N-methylammonium and tri-n-butyl-n-hexylphosphonium.

In formula (3), R⁸, R⁹, and R¹¹ each represent a substituted or unsubstituted hydrocarbon group, preferably a substituted or unsubstituted C₁-C₁₀₀ hydrocarbon group, more preferably a substituted or unsubstituted C₁-C₅₀ hydrocarbon group, and particularly preferably a substituted or unsubstituted C₁-C₃₀ hydrocarbon group.

In another embodiment, R⁸, R⁹, and R¹¹ each represent a substituted or unsubstituted aliphatic hydrocarbon group, alicyclic hydrocarbon group, aromatic hydrocarbon group, or aromatic aliphatic hydrocarbon group, preferably a substituted or unsubstituted C₁-C₁₀₀ aliphatic hydrocarbon group, alicyclic hydrocarbon group, aromatic hydrocarbon group, or aromatic aliphatic hydrocarbon group, preferably a substituted or unsubstituted C₁-C₅₀ aliphatic hydrocarbon group, alicyclic hydrocarbon group, aromatic hydrocarbon group, or aromatic aliphatic hydrocarbon group, and particularly preferably a substituted or unsubstituted C₁-C₃₀ aliphatic hydrocarbon group, alicyclic hydrocarbon group, aromatic hydrocarbon group, or aromatic aliphatic hydrocarbon group.

In formula (3), R¹⁰ represents a hydrogen atom or a substituted or unsubstituted hydrocarbon group, preferably a hydrogen atom or a substituted or unsubstituted C₁-C₁₀₀ hydrocarbon group, more preferably a hydrogen atom or a substituted or unsubstituted C₁-C₅₀ hydrocarbon group, and particularly preferably a hydrogen atom or a substituted or unsubstituted C₁-C₃₀ hydrocarbon group.

In another embodiment, R¹⁰ represents a hydrogen atom or a substituted or unsubstituted aliphatic hydrocarbon group, alicyclic hydrocarbon group, aromatic hydrocarbon group, or aromatic aliphatic hydrocarbon group, preferably a hydrogen atom or a substituted or unsubstituted C₁-C₁₀₀ aliphatic hydrocarbon group, alicyclic hydrocarbon group, aromatic hydrocarbon group, or aromatic aliphatic hydrocarbon group, more preferably a hydrogen atom or a substituted or unsubstituted C₁-C₅₀ aliphatic hydrocarbon group, alicyclic hydrocarbon group, aromatic hydrocarbon group, or aromatic aliphatic hydrocarbon group, and particularly preferably a hydrogen atom or a substituted or unsubstituted C₁-C₃₀ aliphatic hydrocarbon group, alicyclic hydrocarbon group, aromatic hydrocarbon group, or aromatic aliphatic hydrocarbon group.

In formula (3), some or all of R⁸, R⁹, R¹⁰, and R¹¹ may be bonded together to form one or more ring structures. For example, when R⁸ and R¹¹ are bonded together to form a ring structure, the structure can be represented by, for example, the following formula (3a), formula (3b), or formula (3c).

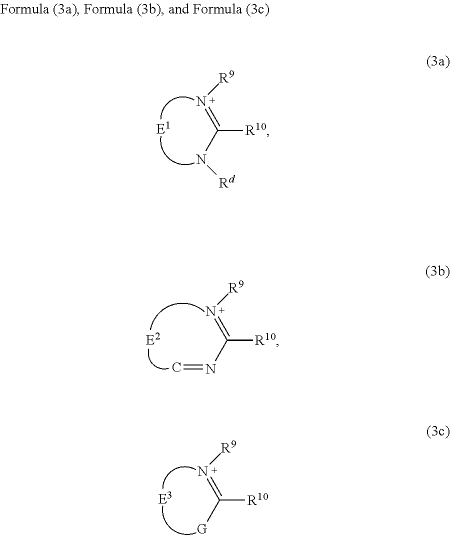

(In the formulas, R⁹ and R¹⁰ are as defined above, R^{d} represents a hydrogen atom or a substituted or unsubstituted hydrocarbon group, E¹, E², and E³ each represent a substituted or unsubstituted divalent hydrocarbon group, and G represents an oxygen atom or a sulfur atom.)

When E¹, E², and E³ each represent a divalent hydrocarbon group having one or more substituents, the substituents are preferably bonded to the carbon atom constituting the ring. The number of substituents bonded to E¹, E², or E³ is one, two, or three.

When E¹, E², and E³ each represent a divalent hydrocarbon group, the divalent hydrocarbon group may be substituted with at least one heteroatom, such as oxygen, nitrogen, or sulfur. When each hydrocarbon group of E¹, E², and E³ is substituted with at least one heteroatom, such as oxygen, nitrogen, or sulfur, the divalent hydrocarbon group has at least one group, such as —O—, —N<, —NH—, or —S—, and the divalent hydrocarbon chain is interrupted by such a group.

In R^{d}, the substituted or unsubstituted hydrocarbon group is preferably a substituted or unsubstituted C₁-C₁₀₀ hydrocarbon group, more preferably a substituted or unsubstituted C₁-C₃₀ hydrocarbon group, and particularly preferably a substituted or unsubstituted C₁-C₃₀ hydrocarbon group.

In another embodiment, when R^{d} represents a substituted or unsubstituted hydrocarbon group, the substituted or unsubstituted hydrocarbon group is a substituted or unsubstituted aliphatic hydrocarbon group, alicyclic hydrocarbon group, aromatic hydrocarbon group, or aromatic aliphatic hydrocarbon group,
preferably a substituted or unsubstituted C₁-C₁₀₀ aliphatic hydrocarbon group, alicyclic hydrocarbon group, aromatic hydrocarbon group, or aromatic aliphatic hydrocarbon group, more preferably a substituted or unsubstituted C₁-C₅₀ aliphatic hydrocarbon group, alicyclic hydrocarbon group, aromatic hydrocarbon group, or aromatic aliphatic hydrocarbon group, and particularly preferably a substituted or unsubstituted C₁-C₃₀ aliphatic hydrocarbon group, alicyclic hydrocarbon group, aromatic hydrocarbon group, or aromatic aliphatic hydrocarbon group.

In E¹, E², and E³, the substituted or unsubstituted divalent hydrocarbon group is preferably a substituted or unsubstituted C₁-C₂₀ divalent hydrocarbon group, more preferably substituted or unsubstituted C₁-C₁₂ divalent hydrocarbon group, and particularly preferably a substituted or unsubstituted C₁-C₆ divalent hydrocarbon group.

In another embodiment, when E¹, E², and E³ each represent a substituted or unsubstituted divalent hydrocarbon group, the substituted or unsubstituted divalent hydrocarbon group is a substituted or unsubstituted divalent aliphatic hydrocarbon group, alicyclic hydrocarbon group, aromatic hydrocarbon group, or aromatic aliphatic hydrocarbon group, preferably a substituted or unsubstituted C₁-C₁₂ divalent aliphatic hydrocarbon group, alicyclic hydrocarbon group, aromatic hydrocarbon group, or aromatic aliphatic hydrocarbon group, more preferably substituted or unsubstituted C₁-C₁₂ divalent aliphatic hydrocarbon group, alicyclic hydrocarbon group, aromatic hydrocarbon group, or aromatic aliphatic hydrocarbon group, and particularly preferably a substituted or unsubstituted C₁-C₅ divalent aliphatic hydrocarbon group, alicyclic hydrocarbon group, aromatic hydrocarbon group, or aromatic aliphatic hydrocarbon group.

Although the above is an example of a case in which R⁸ and R¹¹ are bonded together to form a ring structure, a ring structure can also be formed in a similar manner in cases in which R⁸ and R⁹, R⁹ and R¹⁰, and R¹⁰ and R¹¹ are bonded together to form a ring structure.

The organic cation represented by formula (3) is preferably an organic cation represented by formula (3-1) or formula (3-2).

In formula (3-1), R⁹ and R¹² each represent a substituted or unsubstituted hydrocarbon group, preferably a substituted or unsubstituted C₁-C₁₀₀ hydrocarbon group, more preferably a substituted or unsubstituted C₁-C₅₀ hydrocarbon group, and particularly preferably a substituted or unsubstituted C₁-C₃₀ hydrocarbon group.

In another embodiment, R⁹ and R¹² each represent a substituted or unsubstituted aliphatic hydrocarbon group, alicyclic hydrocarbon group, aromatic hydrocarbon group, or aromatic aliphatic hydrocarbon group,
preferably a substituted or unsubstituted C₁-C₁₀₀ aliphatic hydrocarbon group, alicyclic hydrocarbon group, aromatic hydrocarbon group, or aromatic aliphatic hydrocarbon group, more preferably a substituted or unsubstituted C₁-C₅₀ aliphatic hydrocarbon group, alicyclic hydrocarbon group, aromatic hydrocarbon group, or aromatic aliphatic hydrocarbon group, and particularly preferably a substituted or unsubstituted C₁-C₃₀ aliphatic hydrocarbon group, alicyclic hydrocarbon group, aromatic hydrocarbon group, or aromatic aliphatic hydrocarbon group.

In formula (3-1), R¹⁰, R¹³, and R¹⁴ each represent a hydrogen atom or a substituted or unsubstituted hydrocarbon group, preferably a hydrogen atom or a substituted or unsubstituted C₁-C₁₀₀ hydrocarbon group, more preferably a hydrogen atom or a substituted or unsubstituted C₁-C₅₀ hydrocarbon group, and particularly preferably a hydrogen atom or a substituted or unsubstituted C₁-C₃₀ hydrocarbon group.

In another embodiment, R¹⁰, R¹³, and R¹⁴ each represent a hydrogen atom or a substituted or unsubstituted aliphatic hydrocarbon group, alicyclic hydrocarbon group, aromatic hydrocarbon group, or aromatic aliphatic hydrocarbon group, preferably a hydrogen atom or a substituted or unsubstituted C₁-C₁₀₀ aliphatic hydrocarbon group, alicyclic hydrocarbon group, aromatic hydrocarbon group, or aromatic aliphatic hydrocarbon group, more preferably a hydrogen atom or a substituted or unsubstituted C₁-C₅₀ aliphatic hydrocarbon group, alicyclic hydrocarbon group, aromatic hydrocarbon group, or aromatic aliphatic hydrocarbon group, and
particularly preferably a hydrogen atom or a substituted or unsubstituted C₁-C₃₀ aliphatic hydrocarbon group, alicyclic hydrocarbon group, aromatic hydrocarbon group, or aromatic aliphatic hydrocarbon group.

Specific examples of the organic cation represented by formula (3-1) include 1,3-dimethylimidazolium, 1-ethyl-3-methylimidazolium, 1-methyl-3-propylimidazolium, 1-butyl-3-methylimidazolium, 1-methyl-3-pentylimidazolium, 1-hexyl-3-methylimidazolium, 1-heptyl-3-methylimidazolium, 1-methyl-3-octylimidazolium, 1-methyl-3-nonylimidazolium, 1-decyl-3-methylimidazolium, 1-allyl-3-methylimidazolium, 1-allyl-3-ethylimidazolium, 1-(2-methoxyethyl)-3-methylimidazolium, 1-(2-ethoxyethyl)-3-methylimidazolium, 1-ethyl-3-(2-methoxyethyl)imidazolium, 1-ethyl-3-(2-ethoxyethyl)imidazolium, 1,3-di(tert-butyl)imidazolium, 1,3-bis(1,1-dimethylethyl)imidazolium, 1,3-bis(1,1-dimethylpropyl) imidazolium, 1,3-bis(1,1,3,3-tetramethylbutyl)imidazolium, 1,3-bis(1-methyl-1-phenylethyl)imidazolium, 1,3-bis(1,1-dimethyl-2-phenylethyl)imidazolium, 1,3-bis(1-adamantyl)imidazolium, and the like. Preferred are 1,3-di(tert-butyl)imidazolium, 1,3-bis(1,1-dimethylethyl)imidazolium, 1,3-bis(1,1-dimethylpropyl)imidazolium, 1,3-bis(1,1,3,3-tetramethylbutyl)imidazolium, 1,3-bis(1-methyl-1-phenylethyl)imidazolium, 1,3-bis(1,1-dimethyl-2-phenylethyl)imidazolium, 1,3-bis(1-adamantyl)imidazolium, and particularly preferred is 1,3-di(tert-butyl)imidazolium.

In formula (3-2), R¹⁰, R¹⁵, R¹⁶, R¹⁷, and R¹⁸ each represent a hydrogen atom or a substituted or unsubstituted hydrocarbon group, preferably a hydrogen atom or a substituted or unsubstituted C₁-C₁₀₀ hydrocarbon group, more preferably a hydrogen atom or a substituted or unsubstituted C₁-C₅₀ hydrocarbon group, and particularly preferably a hydrogen atom or a substituted or unsubstituted C₁-C₃₀ hydrocarbon group.

In another embodiment, R¹⁰, R¹⁵, R¹⁶, R¹⁷, and R¹⁸ each represent a hydrogen atom or a substituted or unsubstituted aliphatic hydrocarbon group, alicyclic hydrocarbon group, aromatic hydrocarbon group, or aromatic aliphatic hydrocarbon group,
preferably a hydrogen atom or a substituted or unsubstituted C₁-C₁₀₀ aliphatic hydrocarbon group, alicyclic hydrocarbon group, aromatic hydrocarbon group, or aromatic aliphatic hydrocarbon group,
more preferably a hydrogen atom or a substituted or unsubstituted C₁-C₅₀ aliphatic hydrocarbon group, alicyclic hydrocarbon group, aromatic hydrocarbon group, or aromatic aliphatic hydrocarbon group, and
particularly preferably a hydrogen atom or a substituted or unsubstituted C₁-C₃₀ aliphatic hydrocarbon group, alicyclic hydrocarbon group, aromatic hydrocarbon group, or aromatic aliphatic hydrocarbon group.

Specific examples of the organic cation represented by formula (3-2) include 1-methylpyridinium, 1-ethylpyridinium, 1-propylpyridinium, 1-butylpyridinium, 1-pentylpyridinium, 1-hexylpyridinium, 1-heptylpyridinium, 1-octylpyridinium, 1-nonylpyridinium, 1-decylpyridinium, 1-hexadecylpyridinium, 1-allylpyridinium, 1-(2-methoxyethyl)pyridinium, 1-(2-ethoxyethyl)pyridinium, and the like. Preferred is 1-hexadecylpyridinium.

A, A¹, A², R¹ to R¹⁸, and R^{1a} may form a ring structure together with the carbon atom, oxygen atom, nitrogen atom, or phosphorus atom to which they are bonded.

For example, when R¹³ and R¹⁴ or R¹⁷ and R¹⁸ form a ring structure together with the carbon atom, oxygen atom, nitrogen atom, or phosphorus atom to which they are bonded, for example, a benzimidazolium ring structure or a quinolinium ring structure shown in the following formula (3-1a) or formula (3-2a) can be formed.

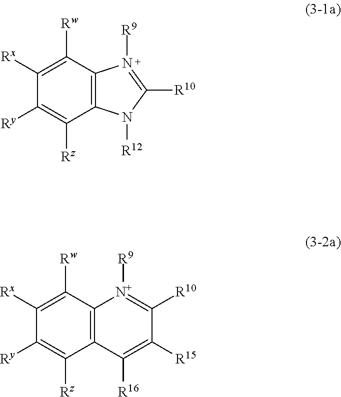

(In the formulas, R⁹, R¹⁰, R¹², R¹⁵, and R¹⁶ are as defined above. R^{x}, R^{x}, R^{y}, and R^{z} each represent a hydrogen atom or a C₁-C₂₀ hydrocarbon group, and R⁹ represents an adamantyl group, and may be, in particular, a 1-adamantyl group.)

Although the above is an example in which R¹³ and R¹⁴ or R¹⁷ and R¹⁸ form a ring structure, a ring structure can also be formed in a similar manner in cases of R¹ and R², R⁴ and R⁵, R⁸ and R⁹, R⁹ and R¹⁰, R¹⁰ and R¹², R¹² and R¹³, R⁹ and R¹⁴, R¹⁰ and R¹⁵, and R¹⁵ and R¹⁶.

Specific examples of formula (1) include the following.

TABLE 1

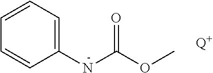

| Q⁺ | Compound No. |
|---|---|
| N,N,N-trimethyl-N-(2-methoxyethyl)ammonium | a-i-1 |
| N,N-diethyl-N-(2-methoxyethyl)-N-methylammonium | a-i-2 |
| N,N,N-triethyl-N-(2-ethoxyethyl)ammonium | a-i-3 |
| N,N-diethyl-N-propyl-N-(2-ethoxyethyl)ammonium | a-i-4 |
| N-ethyl-N-[2-(2-methoxyethoxy)ethyl]- | a-i-5 |
| N,N-dimethylammonium | |
| N,N-diethyl-N-[2-(2-methoxyethoxy) | a-i-6 |
| ethyl]-N-methylammonium | |
| N,N-di(2-methoxyethyl)-N,N-dimethylammonium | a-i-7 |
| N,N-di(2-ethoxyethyl)-N,N-dimethylammonium | a-i-8 |
| N-[2-(2-methoxyethoxy)ethyl]-N-(2-methoxyethyl)- | a-i-9 |
| N,N-dimethylammonium | |
| N-(2-ethoxyethyl)-N-[2-(2-methoxyethoxy)ethyl]- | a-i-10 |
| N,N-dimethylammonium | |
| tri-n-butyl-n-hexylphosphonium | a-i-11 |
| 1,3-di(tert-butyl)imidazolium | a-i-12 |
| 1,3-bis(1,1-dimethylethyl)imidazolium | a-i-13 |
| 1,3-bis(1,1-dimethylpropyl)imidazolium | a-i-14 |
| 1,3-bis(1,1,3,3-tetramethylbutyl)imidazolium | a-i-15 |
| 1,3-bis(1-methyl-i-phenylethyl)imidazolium | a-i-16 |
| 1,3-bis(1,1-dimethyl-2-phenylethyl)imidazolium | a-i-17 |
| 1,3-bis(1-adamantyl)imidazolium | a-i-18 |
| 1-hexadecylpyridinium | a-i-19 |

TABLE 2

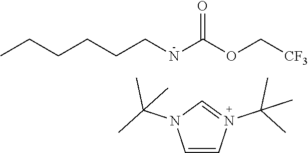

| Q⁺ | Compound No. |
|---|---|
| N,N,N-trimethyl-N-(2-methoxyethyl)ammonium | a-ii-1 |
| N,N-diethyl-N-(2-methoxyethyl)-N-methylammonium | a-ii-2 |
| N,N,N-triethyl-N-(2-ethoxyethyl)ammonium | a-ii-3 |
| N,N-diethyl-N-propyl-N-(2-ethoxyethyl)ammonium | a-ii-4 |
| N-ethyl-N-[2-(2-methoxyethoxy)ethyl]-N,N- | a-ii-5 |
| dimethylammonium | |
| N,N-diethyl-N-[2-(2-methoxyethoxy)ethyl]-N- | a-ii-6 |
| methylammonium | |
| N,N-di(2-methoxyethyl)-N,N-dimethylammonium | a-ii-7 |
| N,N-di(2-ethoxyethyl)-N,N-dimethylammonium | a-ii-8 |
| N-[2-(2-methoxyethoxy)ethyl]-N-(2-methoxyethyl)- | a-ii-9 |
| N,N-dimethylammonium | |
| N-(2-ethoxyethyl)-N-[2-(2-methoxyethoxy)ethyl]- | a-ii-10 |
| N,N-dimethylammonium | |
| tri-n-butyl-n-hexylphosphonium | a-ii-11 |
| 1,3-di(tert-butyl)imidazolium | a-ii-12 |
| 1,3-bis(1,1-dimethylethyl)imidazolium | a-ii-13 |
| 1,3-bis(1,1-dimethylpropyl)imidazolium | a-ii-14 |
| 1,3-bis(1,1,3,3-tetramethylbutyl)imidazolium | a-ii-15 |
| 1,3-bis(1-methyl-i-phenylethyl)imidazolium | a-ii-16 |
| 1,3-bis(1,1-dimethyl-2-phenylethyl)imidazolium | a-ii-17 |
| 1,3-bis(1-adamantyl)imidazolium | a-ii-18 |
| 1-hexadecylpyridinium | a-ii-19 |

TABLE 3

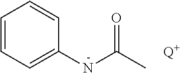

| Q⁺ | Compound No. |
|---|---|
| N,N,N-trimethyl-N-(2-methoxyethyl)ammonium | a-iii-1 |
| N,N-diethyl-N-(2-methoxyethyl)-N-methylammonium | a-iii-2 |
| N,N,N-triethyl-N-(2-ethoxyethyl)ammonium | a-iii-3 |
| N,N-diethyl-N-propyl-N-(2-ethoxyethyl)ammonium | a-iii-4 |
| N-ethyl-N-[2-(2-methoxyethoxy)ethyl]-N, | a-iii-5 |
| N-dimethylammonium | |
| N,N-diethyl-N-[2-(2-methoxyethoxy)ethyl]- | a-iii-6 |
| N-methylammonium | |
| N,N-di(2-methoxyethyl)-N,N-dimethylammonium | a-iii-7 |
| N,N-di(2-ethoxyethyl)-N,N-dimethylammonium | a-iii-8 |
| N-[2-(2-methoxyethoxy)ethyl]-N-(2-methoxyethyl)- | a-iii-9 |
| N,N-dimethylammonium | |
| N-(2-ethoxyethyl)-N-[2-(2-methoxyethoxy)ethyl]-N,N- | a-iii-10 |
| dimethylammonium | |
| tri-n-butyl-n-hexylphosphonium | a-iii-11 |
| 1,3-di(tert-butyl)imidazolium | a-iii-12 |
| 1,3-bis(1,1-dimethylethyl)imidazolium | a-iii-13 |
| 1,3-bis(1,1-dimethylpropyl)imidazolium | a-iii-14 |
| 1,3-bis(1,1,3,3-tetramethylbutyl)imidazolium | a-iii-15 |
| 1,3-bis(1-methyl-i-phenylethyl)imidazolium | a-iii-16 |
| 1,3-bis(1,1-dimethyl-2-phenylethyl)imidazolium, | a-iii-17 |
| 1,3-bis(1-adamantyl)imidazolium | a-iii-18 |
| 1-hexadecylpyridinium | a-iii-19 |

TABLE 4

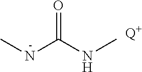

| Q⁺ | Compound No. |
|---|---|
| N,N,N-trimethyl-N-(2-methoxyethyl)ammonium | a-iv-1 |
| N,N-diethyl-N-(2-methoxyethyl)-N-methylammonium | a-iv-2 |
| N,N,N-triethyl-N-(2-ethoxyethyl)ammonium | a-iv-3 |
| N,N-diethyl-N-propyl-N-(2-ethoxyethyl)ammonium | a-iv-4 |
| N-ethyl-N-[2-(2-methoxyethoxy)ethyl]-N,N- | a-iv-5 |
| dimethylammonium | |
| N,N-diethyl-N-[2-(2-methoxyethoxy)ethyl]-N- | a-iv-6 |
| methylammonium | |
| N,N-di(2-methoxyethyl)-N,N-dimethylammonium | a-iv-7 |
| N,N-di(2-ethoxyethyl)-N,N-dimethylammonium | a-iv-8 |
| N-[2-(2-methoxyethoxy)ethyl]-N-(2-methoxyethyl)-N,N- | a-iv-9 |
| dimethylammonium | |
| N-(2-ethoxyethyl)-N-[2-(2-methoxyethoxy)ethyl]- | a-iv-10 |
| N,N-dimethylammonium | |
| tri-n-butyl-n-hexylphosphonium | a-iv-11 |
| 1,3-di(tert-butyl)imidazolium | a-iv-12 |
| 1,3-bis(1,1-dimethylethyl)imidazolium | a-iv-13 |
| 1,3-bis(1,1-dimethylpropyl)imidazolium | a-iv-14 |
| 1,3-bis(1,1,3,3-tetramethylbutyl)imidazolium, | a-iv-15 |
| 1,3-bis(1-methyl-i-phenylethyl)imidazolium | a-iv-16 |
| 1,3-bis(1,1-dimethyl-2-phenylethyl)imidazolium, | a-iv-17 |
| 1,3-bis(1-adamantyl)imidazolium | a-iv-18 |
| 1-hexadecylpyridinium | a-iv-19 |

Preferred specific examples of the onium salt (1) of the present invention are shown below.

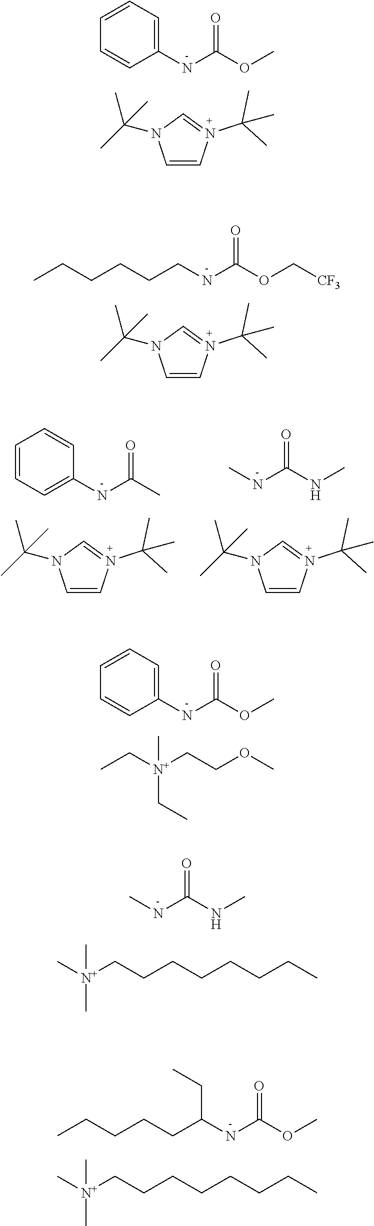

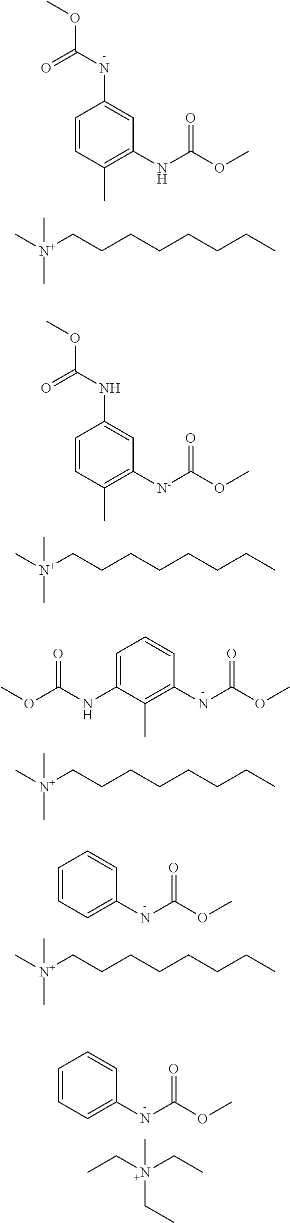

The onium salt (1) can be produced, for example, by a method comprising step 2 described below. A compound represented by formula (6) for use in step 2 (hereinafter referred to as “the carbonate salt (6)”) may be a commercial product or may be obtained by a known method, or can be produced, for example, by a method comprising step 1-1 or step 1-2 described below.

Step 1-1: An onium salt represented by the following formula (5) is reacted with sodium carbonate or potassium carbonate, and the formed sodium chloride or potassium chloride is removed to obtain a carbonate salt represented by the following formula (6-1) (hereinafter referred to as “the carbonate salt (6-1)”). The carbonate salt represented by the following formula (6-1) corresponds to a compound in which R represents a hydrogen atom in formula (6).

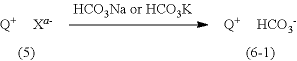

(In the formulas, X^{a} represents a chloride ion, a bromide ion, or an iodine ion. Q⁺ is as defined above)

Step 1-2: A compound represented by the following formula (8) or (9) is reacted with an ester carbonate represented by formula (11) (hereinafter referred to as “the ester carbonate (11)”) to obtain an onium salt represented by the following formula (6-2)) (hereinafter referred to as “the carbonate salt (6-2)”). The onium salt represented by the following formula (6-2) corresponds to a compound in which R represents R⁴ or R⁹ in formula (6).

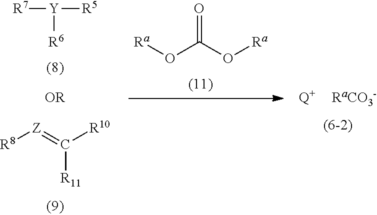

(In the formulas, R^{a} represents R⁴ or R⁹. R⁴, R⁹, R⁶, R⁷, R⁸, R⁹, R¹⁰, R¹¹, Q⁺, Y, and Z are as defined above)

Step 2: An onium salt represented by the following formula (6) is reacted with a compound represented by the following formula (7) to obtain an onium salt represented by formula (1).

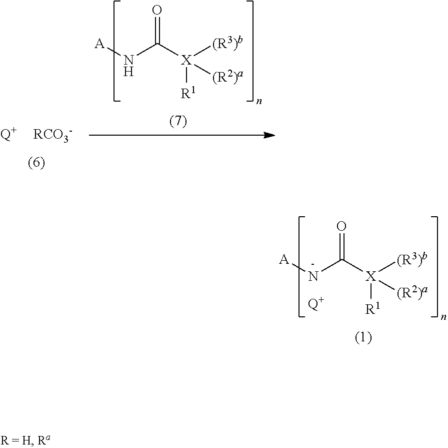

(In the formulas, R¹, R², R³, Q⁺, A, X, a, b, R^{a}, and n are as defined above)

Formula (5):

Q⁺ X^{a-} (5)

(In the formula, Q⁺ is as defined above. X^{a-} represents a chlorine ion, bromide ion, or iodide ion)

### Formula (6):

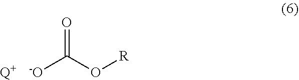

(In the formula, R represents a hydrogen atom, R⁴, or R⁹. Q*, R⁴, and R⁹ are as defined above)

### Formula (7):

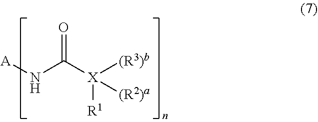

(In the formula, R¹, R², R³, A, a, b, and n are as defined above.)

Step 1-1 is described below.

Specific examples of the onium salt represented by the above formula (5) include N-octyl-N,N,N-trimethylammonium chloride, N,N,N-triethyl-N-methylammuonium chloride, N,N,N-trimethyl-N-(2-methoxyethyl)ammonium chloride, N,N-diethyl-N-(2-methoxyethyl)-N-methylammonium chloride, N,N,N-triethyl-N-(2-ethoxyethyl)ammonium chloride, N,N-diethyl-N-propyl-N-(2-ethoxyethyl)ammonium chloride, N-ethyl-N-[2-(2-methoxyethoxy)ethyl]-N,N-dimethylammonium chloride, N,N-diethyl-N-[2-(2-methoxyethoxy)ethyl]-N-methylammonium chloride, N,N-di(2-methoxyethyl)-N,N-dimethylammonium chloride, N,N-di(2-ethoxyethyl)-N,N-dimethylammonium chloride, N-[2-(2-methoxyethoxy)ethyl]-N-(2-methoxyethyl)-N,N-dimethylammonium chloride, N-(2-ethoxyethyl)-N-[2-(2-methoxyethoxy)ethyl]-N,N-dimethylammonium chloride, tri-n-butyl-n-hexylphosphonium chloride, 1,3-di(tert-butyl)imidazolium chloride, 1,3-bis(1,1-dimethylethyl)imidazolium chloride, 1,3-bis(1,1-dimethylpropyl) imidazolium chloride, 1,3-bis(1,1,3,3-tetramethylbutyl)imidazolium chloride, 1,3-bis(1-methyl-1-phenylethyl)imidazolium chloride, 1,3-bis(1,1-dimethyl-2-phenylethyl)imidazolium chloride, 1,3-bis(1-adamantyl)imidazolium chloride, 1-hexadecylpyridinium chloride, N-octyl-N,N,N-trimethylamnonium bromide, N,N,N-triethyl-N-methylammonium bromide, N,N,N-trimethyl-N-(2-methoxyethyl)ammonium bromide, N,N-diethyl-N-(2-methoxyethyl)-N-methylammonium bromide, N,N,N-triethyl-N-(2-ethoxyethyl)ammonium bromide, N,N-diethyl-N-propyl-N-(2-ethoxyethyl)ammonium bromide, N-ethyl-N-[2-(2-methoxyethoxy)ethyl]-N,N-dimethylammonium bromide, N,N-diethyl-N-[2-(2-methoxyethoxy)ethyl]-N-methylammonium bromide, N,N-di(2-methoxyethyl)-N,N-dimethylammonium bromide, N,N-di(2-ethoxyethyl)-N,N-dimethylammonium bromide, N-[2-(2-methoxyethoxy)ethyl]-N-(2-methoxyethyl)-N,N-dimethylammonium bromide, N-(2-ethoxyethyl)-N-[2-(2-methoxyethoxy)ethyl]-N,N-dimethylammonium bromide, tri-n-butyl-n-hexylphosphonium bromide, 1,3-di(tert-butyl)imidazolium bromide, 1,3-bis(1,1-dimethylethyl)imidazolium bromide, 1,3-bis(1,1-dimethylpropyl) imidazolium bromide, 1,3-bis(1,1,3,3-tetramethylbutyl)imidazolium bromide, 1,3-bis(1-methyl-1-phenylethyl)imidazolium bromide, 1,3-bis(1,1-dimethyl-2-phenylethyl)imidazolium bromide, 1,3-bis(1-adamantyl)imidazolium bromide, 1-hexadecylpyridinium bromide, N-octyl-N,N,N-trimethylammonium iodide, N,N,N-triethyl-N-methylammonium iodide, N,N,N-trimethyl-N-(2-methoxyethyl)ammonium iodide, N,N-diethyl-N-(2-methoxyethyl)-N-methylammonium iodide, N,N,N-triethyl-N-(2-ethoxyethyl)ammonium iodide, N,N-diethyl-N-propyl-N-(2-ethoxyethyl)ammonium iodide, N-ethyl-N-[2-(2-methoxyethoxy)ethyl]-N,N-dimethylammonium iodide, N,N-diethyl-N-[2-(2-methoxyethoxy)ethyl]-N-methylammonium iodide, N,N-di(2-methoxyethyl)-N,N-dimethylammonium iodide, N,N-di(2-ethoxyethyl)-N,N-dimethylammonium iodide, N-[2-(2-methoxyethoxy)ethyl]-N-(2-methoxyethyl)-N,N-dimethylammonium iodide, N-(2-ethoxyethyl)-N-[2-(2-methoxyethoxy)ethyl]-N,N-dimethylammonium iodide, tri-n-butyl-n-hexylphosphonium iodide, 1,3-di(tert-butyl)imidazolium iodide, 1,3-bis(1,1-dimethylethyl)imidazolium iodide, 1,3-bis(1,1-dimethylpropyl) imidazolium iodide, 1,3-bis(1,1,3,3-tetramethylbutyl)imidazolium iodide, 1,3-bis(1-methyl-1-phenylethyl)imidazolium iodide, 1,3-bis(1,1-dimethyl-2-phenylethyl)imidazolium iodide, 1,3-bis(1-adamantyl) imidazolium iodide, 1-hexadecylpyridinium iodide, and the like.

Specific examples of the carbonate salt represented by formula (6-1) corresponding to a compound in which R represents a hydrogen atom in formula (6) include N-octyl-N,N,N-trimethylammonium hydrogen carbonate, N,N,N-triethyl-N-methylammonium hydrogen carbonate, N,N,N-trimethyl-N-(2-methoxyethyl)ammonium hydrogen carbonate, N,N-diethyl-N-(2-methoxyethyl)-N-methylannonium hydrogen carbonate, N,N,N-triethyl-N-(2-ethoxyethyl)amnonium hydrogen carbonate, N,N-diethyl-N-propyl-N-2-ethoxyethyl)ammonium hydrogen carbonate, N-ethyl-N-[2-(2-methoxyethoxy)ethyl]-N,N-dimethylammonium hydrogen carbonate, N,N-diethyl-N-(2-(2-methoxyethoxy)ethyl)-N-methylammonium hydrogen carbonate, N,N-di(2-methoxyethyl)-N,N-dimethylammonium hydrogen carbonate, N,N-di(2-ethoxyethyl)-N,N-dimethylammonium hydrogen carbonate, N-[2-(2-methoxyethoxy)ethyl]-N-(2-methoxyethyl)-N,N-dimethylammonium hydrogen carbonate, N-(2-ethoxyethyl)-N-[2-(2-methoxyethoxy)ethyl]-N,N-dimethylammonium hydrogen carbonate, tri-n-butyl-n-hexylphosphonium hydrogen carbonate, 1,3-di(tert-butyl)imidazolium hydrogen carbonate, 1,3-bis(1,1-dimethylethyl)imidazolium hydrogen carbonate, 1,3-bis(1,1-dimethylpropyl) imidazolium hydrogen carbonate, 1,3-bis(1,1,3,3-tetramethylbutyl)imidazolium hydrogen carbonate, 1,3-bis(1-methyl-1-phenylethyl)imidazolium hydrogen carbonate, 1,3-bis(1,1-dimethyl-2-phenylethyl)imidazolium hydrogen carbonate, 1,3-bis(1-adamantyl)imidazolium hydrogen carbonate, 1-hexadecylpyridinium hydrogen carbonate, and the like.

Sodium carbonate or potassium carbonate is usually used in an amount of 1 mol or more, preferably 1 to 2 mol, and particularly preferably 1 to 1.5 mol, per mole of the onium salt represented by formula (5).

The reaction temperature is usually 0° C. to 100° C. or the boiling temperature of the solvent, preferably 10° C. to 100° C., and more preferably 20° C. to 80° C.

The reaction time is usually 1 to 100 hours, and preferably 1 to 20 hours.

Examples of solvents include tetrahydrofuran, ethyl acetate, methanol, ethanol, acetonitrile, toluene, acetone, and the like. The amount of the solvent when used is usually 100 parts by mass or less, and preferably 0.1 to 50 parts by mass, per part by mass of the compound represented by formula (4). The solvents may be a mixture of two or more different types of solvents, if necessary.

In step 1-1, the reaction may be performed, if necessary, in an inert gas atmosphere, such as nitrogen, argon, or helium, which do not affect the reaction.

The obtained carbonate salt (6-1) can be purified according to common methods, such as concentration and recrystallization, or can be used as a raw material for step 2 without purification.

The onium salt represented by formula (5) may be a commercial product. The onium salt represented by formula (5) for use may be obtained, for example, by the known method described below.

The onium salt represented by formula (5) can be obtained by reacting a compound represented by the following formula (8) or formula (9) with a compound represented by the following formula (10).

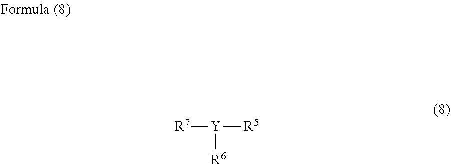

(In the formula, R⁵, R⁶, and R⁷ are as defined above. Y represents a nitrogen atom or a phosphorus atom)

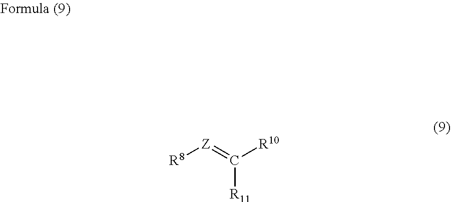

(In the formula, R⁸, R¹⁰, and R¹¹ are as defined above. Z represents a nitrogen atom or a phosphorus atom)

R^{a}X^{a} Formula (10)

(In the formula, R^{a} represents R⁴ or R⁹, R⁴, R⁹, and X^{a} are as defined above.)

In formula (8), Y represents a nitrogen atom or a phosphorus atom, and preferably a nitrogen atom.

Specific examples of the compound represented by the above formula (8) include
dimethyl-n-octylamine,
triethylamine,
N,N-dimethyl-N-(2-methoxyethyl)amine,
N, N-diethyl-N-(2-methoxyethyl)amine,
N-ethyl-N-(2-methoxyethyl)-N-methylamine,
N,N-diethyl-N-(2-ethoxyethyl)amine,
N-ethyl-N-propyl-N-(2-ethoxyethyl)amine,
N,N-diethyl-N-(2-ethoxyethyl)ammonium,
N-ethyl-N-[2-(2-methoxyethoxy)ethyl]-N-methylamine,
N-[2-(2-methoxyethoxy)ethyl]-N, N-dimethylamine,
N,N-diethyl-N-[2-(2-methoxyethoxy)ethyl]amine,
N-ethyl-N-[2-(2-methoxyethoxy)ethyl]-N-methylamine,
N,N-di(2-methoxyethyl)-N,N-dimethylamine,
N,N-di(2-ethoxyethyl)-N-methylamine,
N-[2-(2-methoxyethoxy)ethyl]-N-(2-methoxyethyl)-N-methylamine,
N-(2-ethoxyethyl)-N-[2-(2-methoxyethoxy)ethyl]-N-methylamine,
di-n-butyl-n-hexylphosphine, and the like.

In formula (9), Z represents a nitrogen atom or a phosphorus atom, and preferably a nitrogen atom. The compound represented by formula (9) is preferably a compound represented by the following formula (9-1) or formula (9-2).

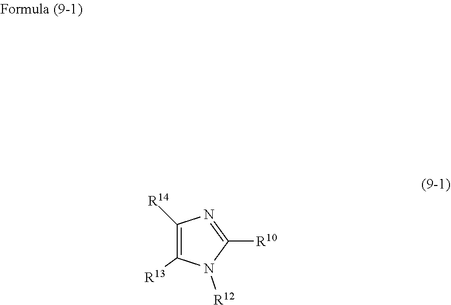

(In the formula, R¹⁰, R¹², R¹³, and R¹⁴ are as defined above)

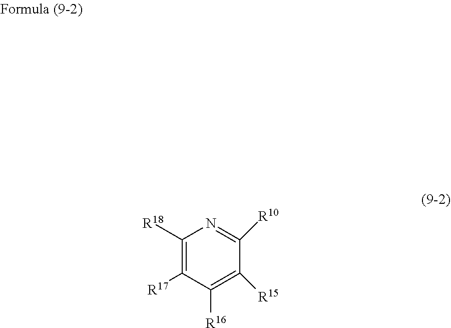

(In the formula, R¹⁰, R¹⁵, R¹⁶, R¹⁷, and R¹⁸ are as defined above.) Specific examples of the compound represented by formula (9-1) include 1-methylimidazole, 1-ethylimidazole, 1-propylimidazole, 1-butyl-3-methylimidazole, 1-pentylimidazole, 1-hexylimidazole, 1-heptyliimidazole, 1-octylimidazole, 1-nonylimidazole, 1-decylimidazole, 1-allylimidazole, 1-(2-methoxyethyl)imidazole, 1-(2-ethoxyethyl)imidazole, 1-(tert-butyl)imidazole, 1-(1,1-dimethylethyl)imidazole, 1-(1,31-dimethylpropyl) imidazole, 1-(1,1,3,3-tetramethylbutyl)imidazole, 1-(1-methyl-1-phenylethyl)imidazole, 1-(1,1-dimethyl-2-phenylethyl)imidazole, 1-(1-adamantyl)imidazole, and the like. Preferred are 1-(tert-butyl)imdidazole, 1-(1,1-dimethylethyl)imidazole, 1-(1,1-dimethylpropyl) imidazole, 1-(1,1,3,3-tetramethylbutyl)imidazole, 1-(1-methyl-1-phenylethyl)imidazole, 1-(1,1-dimethyl-2-phenylethyl)imidazole, and 1-(1-adamantyl)imidazole.

Specific examples of the compound represented by formula (9-2) include pyridine, 2-methylpyridine, 3-methylpyridine, 4-methylpyridine, α-picoline, β-picoline, γ-picoline, 2-ethylpyridine, 3-ethylpyridine, 4-ethylpyridine, 2-propylpyridine, 3-propylpyridine, 4-propylpyridine, 2-butylpyridine, 3-butylpyridine, 4-butylpyridine, 2-tert-butylpyridine, 3-tert-butylpyridine, 4-tert-butylpyridine, 2-amylpyridine, 3-amylpyridine, 4-amylpyridine, 2-chloropyridine, 3-chloropyridine, 4-chloropyridine, 2-fluoropyridine, 3-fluoropyridine, 4-fluoropyridine, 2,3-lutidine, 2,4-lutidine, 2,5-lutidine, 2,6-lutidine, 3,4-lutidine, 3,5-lutidine, 2,4,6-collidine, 2,3,5-collidine, 2-cyanopyridine, 3-cyanopyridine, 4-cyanopyridine, 2-acetylpyridine, 3-acetylpyridine, 4-acetylpyridine, 2-methoxypyridine, 3-methoxypyridine, 4-methoxypyridine, quinoline, isoquinoline, 1-methylisoquinoline, and the like. Preferred is pyridine.

Step 1-2 is described below.

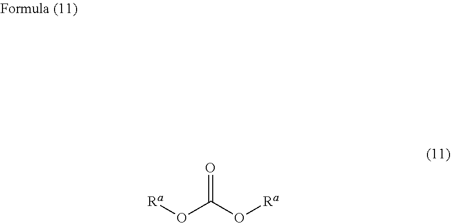

(In the formula, R^{a} represents R⁴ or R¹. R¹ and R⁹ are as defined above.)

Specific examples of the ester carbonate (11) include dialkyl carbonates, such as dimethyl carbonate, diethyl carbonate, dipropyl carbonate, dibutyl carbonate, dipentyl carbonate, and dihexyl carbonate; and alkylene carbonates, such as ethylene carbonate, propylene carbonate, and butylene carbonate. Preferred among these are dimethyl carbonate, diethyl carbonate, dipropyl carbonate, and dibutyl carbonate, and particularly preferred is dimethyl carbonate.

Specific examples of the carbonate salt represented by formula (6-2) corresponding to a compound in which R represents R^{a} or R⁹ in formula (6) include N-octyl-N, N,N-trimethylamionium methyl carbonate, N,N,N-triethyl-N-methylammonium methyl carbonate, N,N,N-trimethyl-N-(2-methoxyethyl)ammonium methyl carbonate, N,N-diethyl-N-(2-methoxyethyl)-N-methylammonium methyl carbonate, N,N-diethyl-N-(2-methoxyethyl)-N-methylamonriium ethyl carbonate, N,N,N-triethyl-N-(2-ethoxyethyl)amnonium ethyl carbonate, N,N-diethyl-N-propyl-N-(2-ethoxyethyl)ammonium ethyl carbonate, N,N-diethyl-N-propyl-N-(2-ethoxyethyl)ammonium propyl carbonate, N-ethyl-N-[2-(2-methoxyethoxy)ethyl]-N,N-dimethylammonium methyl carbonate, N-ethyl-N-[2-(2-methoxyethoxy)ethyl]-N,N-dimethylammonium ethyl carbonate, N,N-diethyl-N-[2-(2-methoxyethoxy)ethyl]-N-methylammonium methyl carbonate, N,N-diethyl-N-[2-(2-methoxyethoxy)ethyl]-N-methylammonium ethyl carbonate, N,N-di(2-methoxyethyl)-N,N-dimethylammonium methyl carbonate, N,N-di(2-ethoxyethyl)-N,N-dimethylammonium methyl carbonate, N-[2-(2-methoxyethoxy)ethyl]-N-(2-methoxyethyl)-N,N-dimethylammonium methyl carbonate, N-(2-ethoxyethyl)-N-2-(2-methoxyethoxy)ethyl)-N,N-dimethylammonium methyl carbonate, tri-n-butyl-n-hexylphosphonium butyl carbonate, and the like.

In the reaction, the ester carbonate represented by formula (11) is usually used in an amount of 0.8 mol to an excessive amount, and preferably 1.0 to 1.5 mol, per mole of the compound represented by formula (8) or formula (9).

The reaction temperature is usually 10° C. to 100° C. or the boiling temperature of the solvent.

The reaction time is usually 1 to 100 hours, and preferably 1 to 10 hours.

Solvents may or may not be used. The solvent for use may be the ester carbonate (11) in an excessive amount, or may be a solvent, such as tetrahydrofuran, ethyl acetate, acetonitrile, toluene, or acetone. The amount of the solvent when used is usually 100 parts by mass or less, and preferably 0.1 to 50 parts by mass, per part by mass of the compound represented by formula (4). The solvents may be a mixture of two or more different types of solvents, if necessary.

Step 2 is described below.

An onium salt represented by formula (6) is reacted with a compound represented by formula (7) to obtain the onium salt represented by formula (1). During this reaction, CO₂ and water or an alcohol represented by R^{a}OH are removed.

In formula (7), R¹, R², R³, A, a, b, and n are as defined above.

The compound represented by formula (7) is preferably a compound represented by any one of the following formulas (7-1) to (7-4).

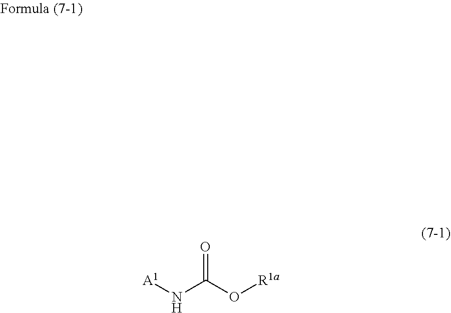

(In the formula, A¹ and R^{1a} are as defined above)

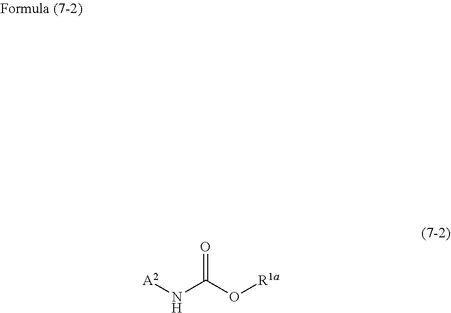

(In the formula, A² and R^{1a} are as defined above)

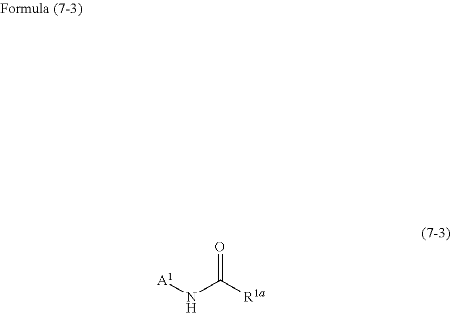

(In the formula, A¹ and R^{1a} are as defined above)

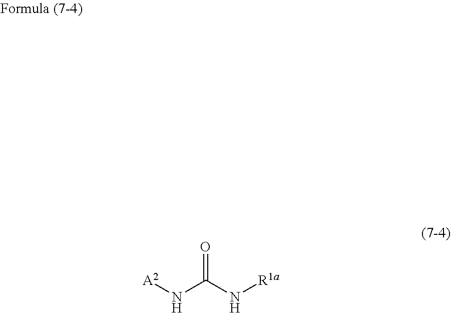

(In the formula, A² and R^{1a} are as defined above.) Specific examples of the compound represented by formula (7) include the following compounds. In the following specific examples, Ph represents a phenyl group, Me represents a methyl group, Et represents an ethyl group, Bu represents an n-butyl group, Hex represents an n-hexyl group, cHex represents a cyclohexyl group, and TFEt represents a 1,1,1-trifluoroethyl group.

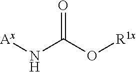

| A^{x} | R^{1x} | Compound No. |
|---|---|---|
| Ph | Me | 7-1-11 |
| Ph | Et | 7-1-12 |
| Ph | Bu | 7-1-13 |
| Ph | Hex | 7-1-14 |
| Ph | TFEt | 7-1-15 |
| Me | Me | 7-2-1 |
| Me | Et | 7-2-2 |
| Me | Bu | 7-2-3 |
| Me | Hex | 7-2-4 |
| Me | TFEt | 7-2-5 |
| Hex | Me | 7-2-6 |
| Hex | Et | 7-2-7 |
| Hex | Bu | 7-2-8 |
| Hex | Hex | 7-2-9 |
| Hex | TFEt | 7-2-10 |
| cHex | Me | 7-2-11 |
| cHex | Et | 7-2-12 |
| cHex | Bu | 7-2-13 |
| cHex | Hex | 7-2-14 |
| cHex | TFEt | 7-2-15 |

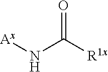

| A^{x} | R^{1x} | Compound No. |
|---|---|---|
| Me | Me | 7-3-1 |
| Me | Et | 7-3-2 |
| Me | Bu | 7-3-3 |
| Me | Hex | 7-3-4 |
| Me | TFEt | 7-3-5 |
| Hex | Me | 7-3-6 |
| Hex | Et | 7-3-7 |
| Hex | Bu | 7-3-8 |
| Hex | Hex | 7-3-9 |
| Hex | TFEt | 7-3-10 |
| cHex | Me | 7-3-11 |
| cHex | Et | 7-3-12 |
| cHex | Bu | 7-3-13 |
| cHex | Hex | 7-3-14 |
| cHex | TFEt | 7-3-15 |
| Ph | Me | 7-3-16 |
| Ph | Bt | 7-3-17 |
| Ph | Bu | 7-3-18 |
| Ph | Hex | 7-3-19 |
| Ph | TFEt | 7-3-20 |

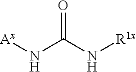

| A^{x} | R^{1x} | Compound No. |
|---|---|---|
| Me | Me | 7-4-1 |
| Me | Et | 7-4-2 |
| Me | Bu | 7-4-3 |
| Me | Hex | 7-4-4 |
| Me | TFEt | 7-4-5 |
| Hex | Me | 7-4-6 |
| Hex | Et | 7-4-7 |
| Hex | Bu | 7-4-8 |
| Hex | Hex | 7-4-9 |
| Hex | TFEt | 7-4-10 |
| cHex | Me | 7-4-11 |
| cHex | Et | 7-4-12 |
| cHex | Bu | 7-4-13 |
| cHex | Hex | 7-4-14 |
| cHex | TFEt | 7-4-15 |
| Ph | Me | 7-4-16 |
| Ph | Et | 7-4-17 |
| Ph | Bu | 7-4-18 |
| Ph | Hex | 7-4-19 |
| Ph | TFEt | 7-4-20 |

Particularly preferred are the following compounds.

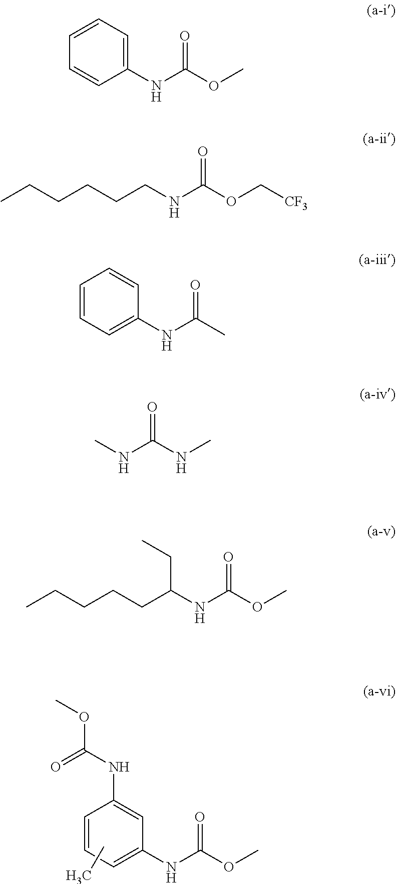

The compound represented by formula (7) is usually used in an amount of about 1 mol, preferably 0.8 to 1.5 mol, and particularly preferably 0.8 to 1.2 mol, per mole of the onium salt represented by formula (6).

The reaction temperature is usually 0 to 100° C. or the boiling point of the solvent, preferably 10 to 80° C., and more preferably 10 to 70° C.

The reaction time is usually 0.5 to 100 hours, and preferably 1 to 10 hours.

Examples of solvents include tetrahydrofuran, ethyl acetate, methanol, ethanol, acetonitrile, toluene, acetone, and the like. The amount of the solvent when used is usually 100 parts by mass or less, and preferably 0.1 to 50 parts by mass, per part by mass of the compound represented by formula (4). The solvents may be a mixture of two or more different types of solvents, if necessary.

In step 1-2, the reaction may be performed, if necessary, in an inert gas atmosphere, such as nitrogen, argon, or helium, which do not affect the reaction.

The obtained carbonate salt (6-2) can be purified according to common methods, such as concentration and recrystallization, or can be used as a raw material for step 2 without purification.

The compound represented by formula (7) may be a commercial product or may be obtained by a known method. Alternatively, for example, (I) when X=N or O, or (II) when X=C, the compound represented by formula (7) can be obtained as described below.

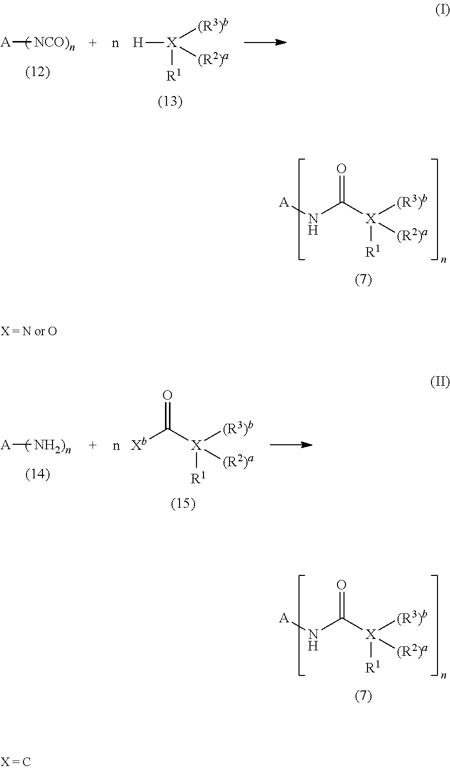

(In the formulas, A, R¹, R², R³, a, b, and n are as defined above. X^{b} represents OH, OCH₃, OC₂H₅, or O-(succinimide).)
(I) When X=N (a nitrogen atom) or O (an oxygen atom), the compound represented by formula (7) can be obtained by using a compound represented by formula (13) in an amount of n ml or an excessive amount per mole of an isocyanate compound represented by formula (12), and, if necessary, in the presence of a solvent, performing a reaction at 10° C. to the boiling temperature of the solvent for 1 to 24 hours. Examples of the solvent include tetrahydrofuran, ethyl acetate, acetonitrile, toluene, acetone, and the like.
(ii) When X=C (a carbon atom), the compound represented by formula (7) can be obtained by using a compound represented by formula (15) in an amount of n mol or an excessive amount per mole of an amine compound represented by formula (14), and, if necessary, in the presence of a solvent, performing a reaction at 10° C. to the boiling temperature of the solvent for 1 to 24 hours. Examples of the solvent include tetrahydrofuran, ethyl acetate, acetonitrile, toluene, acetone, and the like.

Specific examples of the isocyanate compound represented by formula (12) are shown below. However, the present invention is not limited thereto.

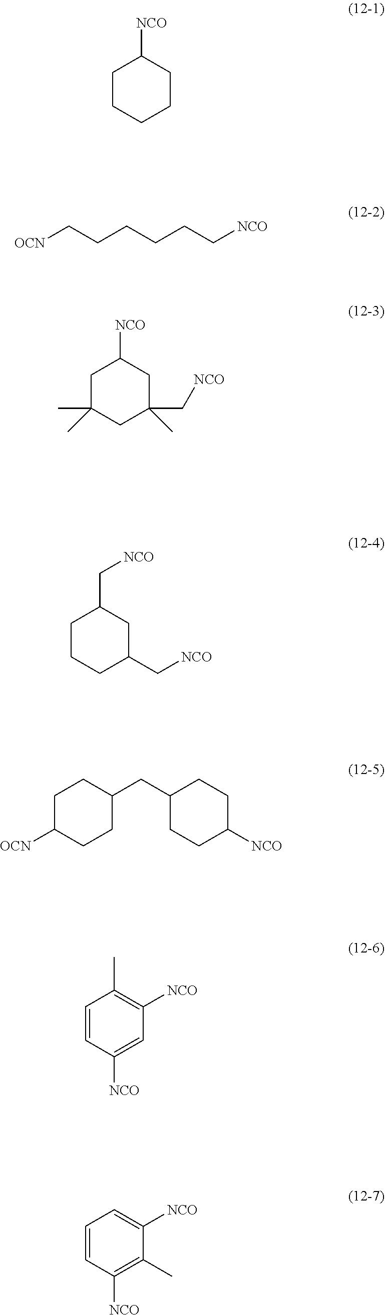

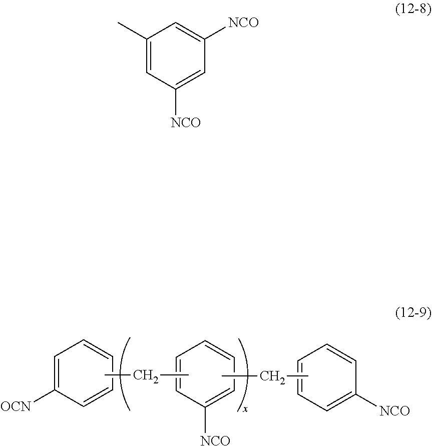

(In formula (12-9), x represents an integer of 0 or more and 20 or less, and preferably 1 or more and 20 or less)

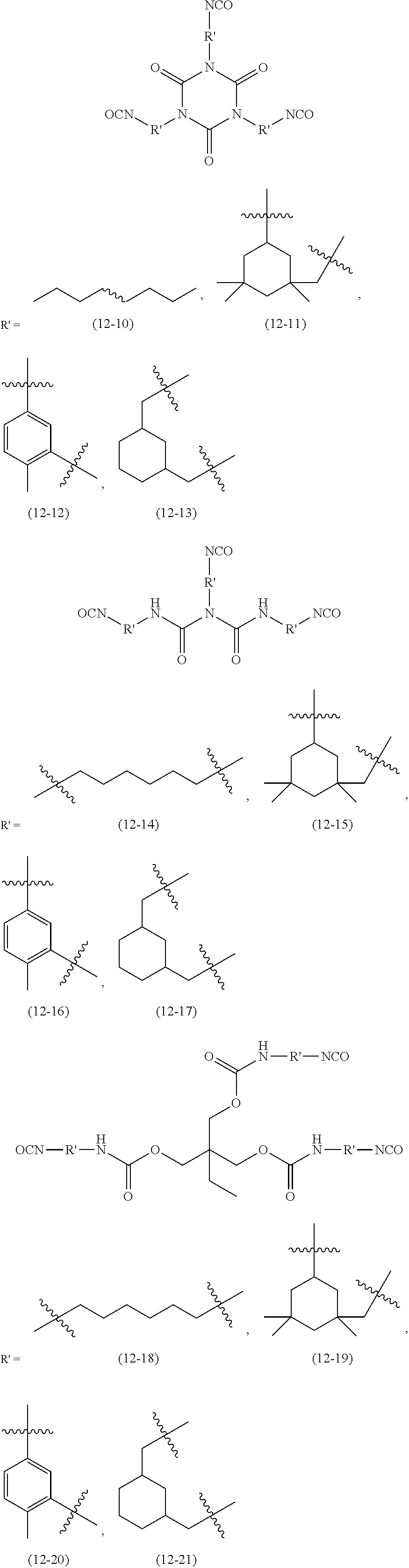

The isocyanate compound represented by formula (12) is preferably a compound represented by (12-1), (12-2), (12-6), or (1.2-14, and particularly preferably (12-1), (12-2), or (12-14).

Specific examples of the compound represented by formula (13) include phenols, such as phenol, xylenol, cresol, resorcinol, nitrophenol, and chlorophenol; oximes, such as acetone oxime, methyl ethyl ketone oxime, and cyclohexanone oxime; alcohols, such as methanol, ethanol, n-propyl alcohol, isopropyl alcohol, n-butyl alcohol, isobutyl alcohol, t-butyl alcohol, t-pentanol, ethylene glycol monoethyl ether, ethylene glycol monobutyl ether, diethylene glycol monoethyl ether, propylene glycol monomethyl ether, and benzyl alcohol; and the like.

Specific examples of the amine compound represented by formula (14) are shown below. However, the present invention is not limited thereto.

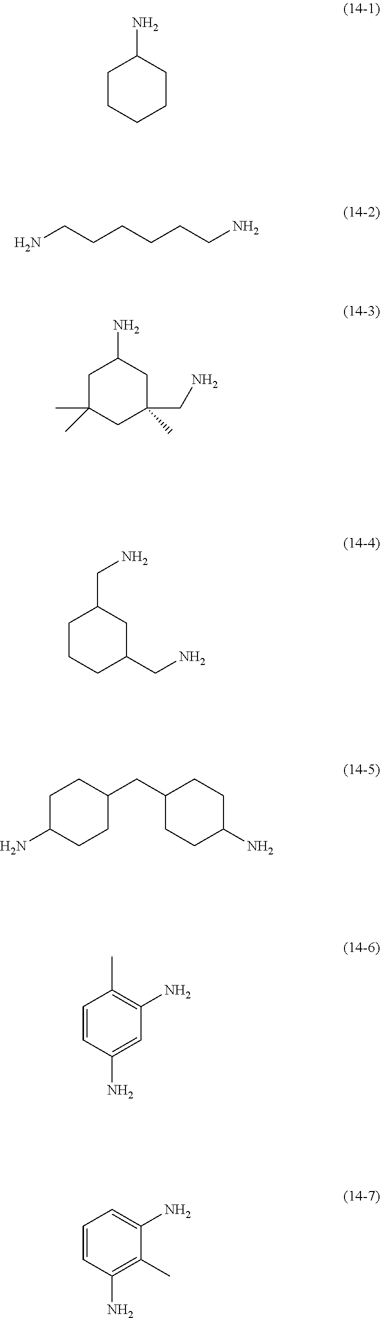

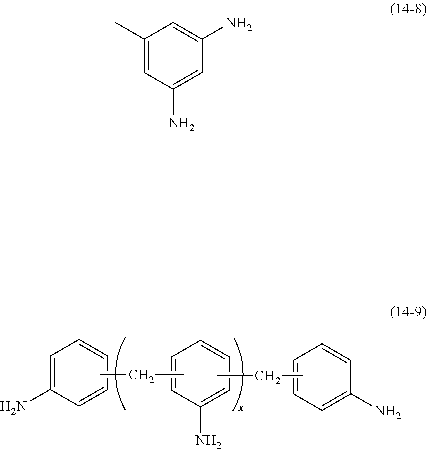

(In formula (14-9), x represents an integer of 0 or more and 20 or less, and preferably 1 or more and 20 or less.)

The amine compound represented by formula (14) is preferably a compound represented by (14-1), (14-2) or (14-6), and particularly preferably (14-1) or (14-2).

Specific examples of the compound represented by formula (15) include carboxylic acids, such as formic acid, acetic acid, propionic acid, butyric acid, pentanoic acid, hexanoic acid, heptanoic acid, octanoic acid, 2-ethylhexanoic acid, capric acid, lauric acid, tetradecyl acid, palmitic acid, octadecyl acid, cyclohexanoic acid, ethoxyacetic acid, propoxyacetic acid, 2-(2-methoxyethoxy)acetic acid, 2-(2-ethoxyethoxy)acetic acid, 2-(2-propoxyethoxy)acetic acid, 3-methoxypropanoic acid, 3-ethoxypropanoic acid, 3-(2-methoxyethoxy)propanoic acid, 3-(2-ethoxyethoxy)propanoic acid, 3-(2-propoxyethoxy)propanoic acid, 3-(3-methoxypropoxy)propanoic acid, 3-(3-ethoxypropoxy)propanoic acid, 3-(3-propoxypropoxy)propanoic acid, oleic acid, linoleic acid, sorbic acid, benzoic acid, phthalic acid, isophthalic acid, terephthalic acid, lactic acid, salicylic acid, trifluoroacetic acid, oxalic acid, malonic acid, succinic acid, glutaric acid, sebacic acid, adipic acid, fumaric acid, maleic acid, 1,7-heptanedicarboxylic acid, and 1,10-decanedicarboxylic acid 1,4-cyclohexanedicarboxylic acid. Preferred are formic acid, acetic acid, propionic acid, butyric acid, pentanoic acid, hexanoic acid, heptanoic acid, octanoic acid, and 2-ethylhexanoic acid, and more preferred are acetic acid, and an ester having an OCH₃, OC₂H₅ or O-(succinimide) group derived from the above carboxylic acids.

When the compound represented by formula (6) is an onium salt in which Q° in formula (6) represents the cation represented by formula (3-1), other than the method comprising step 1-1 or step 1-2, the production is also possible, for example, by a method comprising the following step 3.

### Step 3:

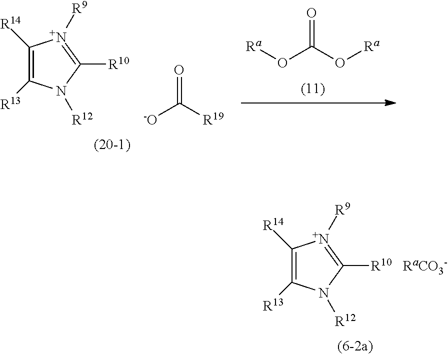

(In the formulas, R^{a}, R⁹, R¹⁰, R¹², R¹³, R¹⁴, and R¹⁹ are as defined above.)

An imidazolium carboxylate represented by formula (20-1) (hereinafter referred to as “the imidazolium carboxylate (20-1)”) is reacted with the ester carbonate (11) to produce a compound in which R represents R^{a}, and Q⁺ represents an organic cation represented by formula (3-1) in formula (6) (hereinafter referred to as “the carbonate salt (6-2a)”). R^{a} is as defined above.

The carbonate salt (6-2a) can be used as a raw material for step 2; however, if necessary, the following step 4 may be further performed to prepare a compound in which R represents a hydrogen atom, and Q⁺ represents an organic cation represented by formula (3-1) in formula (6) (hereinafter referred to as “the carbonate salt (6-1a)”) for use as the carbonate salt (6) in step 2.

### Step 4:

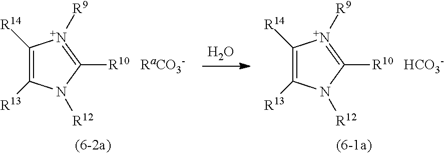

(In the formulas, R^{a}, R⁹, R¹⁰, R¹², R¹³, and R¹⁴ are as defined above.)

The carbonate salt (6-2a) and water are reacted to obtain the carbonate salt (6-1a).

Step 3 is described below.

The imidazolium carboxylate represented by formula (20-1), which is used as a raw material, may be a commercial product or may be produced by a known method, or can be produced by the method described below.

In step 3, the amount of the ester carbonate (11) for use is usually 1 to 10 mol, and preferably 1 to 3 mol, per mole of the imidazolium carboxylate (20-1).

The reaction temperature is usually 10° C. to 120° C. or the boiling temperature of the solvent.

The reaction time is usually 1 to 20 hours, and preferably 1 to 1.0 hours.

The ester carbonate compound (7) may be used in an excessive amount for use as the solvent; alternatively, other solvents may be used. Examples of other solvents include methanol, tetrahydrofuran, ethyl acetate, acetonitrile, toluene, acetone, and the like. The amount of the solvent when used is usually 100 parts by mass or less, and preferably 0.1 to 50 parts by mass, per part by mass of the compound represented by formula (4). The solvents may be a mixture of two or more different types of solvents, if necessary.

The reaction may be performed, if necessary, in an inert gas atmosphere, such as nitrogen, argon, or helium, which do not affect the reaction.

After completion of the reaction, the carbonate salt (6-2a) can be obtained, for example, by removing impurities (e.g., unreacted raw materials) by washing with an organic solvent, or concentrating the reaction liquid, and may be purified by recrystallization, etc., if necessary.

Step 4 is described below.

The amount of water for use is usually 1 mol or more, and preferably 1 to 10 mol, per mole of the carbonate salt (6-2a). Water can also be used in an excessive amount for use as a reaction solvent.

The reaction temperature when the carbonate salt (6-2a) is reacted with water is usually 10° C. or more, preferably 10° C. to 1.00° C., and more preferably 10° C. to 80° C.

The reaction time is usually 0.1 to 10 hours, and preferably 0.1 to 5 hours.

Water can be used as a solvent. When a solvent other than water is used, examples of the solvent include methanol, tetrahydrofuran, ethyl acetate, acetonitrile, toluene, acetone, and the like. The amount of the solvent for use is usually 100 parts by mass or less, and preferably 0.1 to 50 parts by mass, per part by mass of the compound represented by formula (4). The solvents may be a mixture of two or more different types of solvents, if necessary.

The reaction may be performed, if necessary, in an inert, gas atmosphere, such as nitrogen, argon, or helium, which do not affect the reaction.

The obtained carbonate salt. (6-1a) can be purified according to common methods, such as concentration and recrystallization, or can be used as a raw material for step 2 without purification.

The imidazolium carboxylate represented by formula (20-1) for use in step 3 can be produced by the following method.

A dicarbonyl compound represented by the following formula (16), amine compounds represented by the following formula (17x) and formula (17y), an aldehyde represented by the following formula (18), and a monovalent aliphatic carboxylic acid, such as acetic acid, represented by the following formula (19) are reacted to obtain an imidazolium carboxylate represented by the following formula (20-1) (hereinafter referred to as “the imidazolium carboxylate (20-1)¹”).

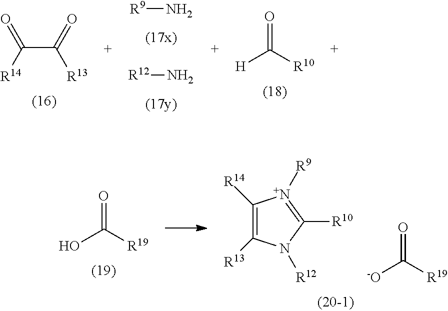

(In the formulas, R⁹, R¹⁰, R¹², R¹³, R¹⁴, and R¹⁹ are as defined above.)

The dicarbonyl compound represented by formula (16) (hereinafter referred to as “the dicarbonyl compound (16)”) is preferably, for example, glyoxal, diacetyl, 3,4-hexanedione, 2,3-pentanedione, 2,3-heptanedione, 5-methyl-2,3-hexanedione, 3-methyl-2,3-cyclopentanedione, 1,2-cyclohexanedione, 1-phenyl-1,2-propanedione, or dibenzoyl, more preferably glyoxal or diacetyl, and even more preferably glyoxal.

Examples of the amine compound represented by formula (17x) (hereinafter referred to as “the amine compound (17x)”) and the amine compound represented by formula (17y) (hereinafter referred to as “the amine compound (17y)”) include tert-butylamine, 1,1,3,3-tetramethylbutylamine, and 1-adamantylamine. Preferred is 1,1,3,3-tetramethylbutylamine.

Examples of the aldehyde represented by formula (18) include aliphatic, alicyclic, or aromatic aldehydes, such as formaldehyde, acetaldehyde, propionaldehyde, and benzaldehyde.

Examples of the monovalent aliphatic carboxylic acid represented by formula (19) (hereinafter referred to as “the aliphatic carboxylic acid (19)”) include carboxylic acids, such as formic acid, acetic acid, propionic acid, butyric acid, pentanoic acid, hexanoic acid, heptanoic acid, octanoic acid, 2-ethylhexanoic acid, capric acid, lauric acid, tetradecyl acid, palmitic acid, octadecyl acid, cyclohexanoic acid, ethoxyacetic acid, propoxyacetic acid, 2-(2-methoxyethoxy)acetic acid, 2-(2-ethoxyethoxy)acetic acid, 2-(2-propoxyethoxy)acetic acid, 3-methoxypropanoic acid, 3-ethoxypropanoic acid, 3-(2-methoxyethoxy)propanoic acid, 3-(2-ethoxyethoxy)propanoic acid, 3-(2-propoxyethoxy)propanoic acid, 3-(3-methoxypropoxy)propanoic acid, 3-(3-ethoxypropoxy)propanoic acid, 3-(3-propoxypropoxy)propanoic acid, oleic acid, linoleic acid, sorbic acid, benzoic acid, phthalic acid, isophthalic acid, terephthalic acid, lactic acid, salicylic acid, and trifluoroacetic acid. Preferred are formic acid, acetic acid, propionic acid, butyric acid, pentanoic acid, hexanoic acid, heptanoic acid, octanoic acid, and 2-ethylhexanoic acid, and more preferred is acetic acid.

Specific examples of the imidazolium carboxylate (20-1) include 1,3-dimethylimidazolium formate, 1-ethyl-3-methylimidazolium formate, 1-butyl-3-methylimidazolium formate, 1-methyl-3-octylimidazolium formate, 1-methyl-3-(1,1,3,3-tetramethylbutyl)imidazolium formate, 1-methyl-3-(2-ethylhexyl)imidazolium formate, 1-dodecyl-3-methylimidazolium formate, 1-methyl-3-octadecylimidazolium formate, 1-benzyl-3-methylimidazolium formate, 1,3-dibutylimidazolium formate, 1-butyl-3-ethylimidazolium formate, 1-butyl-3-octylimidazolium formate, 1-butyl-3-(1,1,3,3-tetramethylbutyl)imidazolium formate, 1-butyl-3-(2-ethylhexylii imidazolium formate, 1-butyl-3-dodecylimidazolium formate, 1-butyl-3-octadecylimidazolium formate, 1-benzyl-3-butylimidazolium formate, 1,3-dioctylimidazolium formate, 1,3-bis(1,1,3,3-tetramethylbutyl)imidazolium formate, 1-ethyl-3-octylimidazolium formate, 1-ethyl-3-(1,1,3,3-tetramethylbutyl)imidazolium formate, 1-octyl-3-(2-ethylhexyl)imidazolium formate, 1-(1,1,3,3-tetramethylbutyl)-3-(2-ethylhexyl) imidazolium formate, 1-dodecyl-3-octylimidazolium formate, 1-dodecyl-3-(1,1,3,3-tetramethylbutyl) imidazolium formate, 1-octyl-3-octadecylimidazolium formate, 1-(1,1,3,3-tetramethylbutyl)-3-octadecylimidazolium formate, 1-benzyl-3-octylimidazolium formate, 1-benzyl-3-(1,1,3,3-tetramethylbutyl)imidazolium formate, 1,3-bis(2-ethylhexyl)imidazolium formate, 1-ethyl-3-(2-ethylhexyl) imidazolium formate, 1-(2-ethylhexyl)-3-dodecylimidazolium formate, 1-(2-ethylhexyl)-3-octadecylimidazolium formate, 1-benzyl-3-(2-ethylhexyl) imidazolium formate, 1,3-didodecylimidazolium formate, 1-dodecyl-3-octadecylimidazolium formate, 1-benzyl-3-dodecylimidazolium formate, 1,3-dioctadecylimidazolium formate, 1-benzyl-3-octadecylimidazolium formate, 1,3-dibenzylimidazolium formate; 1,3-dimethylimidazolium acetate, 1-ethyl-3-methylimidazolium acetate, 1-butyl-3-methylimidazolium acetate, 1-methyl-3-octylimidazolium acetate, 1-methyl-3-(1,1,3,3-tetramethylbutyl)imidazoliurm acetate, 1-methyl-3-(2-ethylhexyl)imidazolium acetate, 1-dodecyl-3-methylimidazolium acetate, 1-methyl-3-octadecylimidazolium acetate, 1-benzyl-3-methylimidazolium acetate, 1,3-dibutylimidazolium acetate, 1-butyl-3-ethylimidazolium acetate, 1-butyl-3-octylimidazolium acetate, 1-butyl-3-(1,1,3,3-tetramethylbutyl)imidazolium acetate, 1-butyl-3-(2-ethylhexyl)imidazolium acetate, 1-butyl-3-dodecylimidazolium acetate, 1-butyl-3-octadecylimidazolium acetate, 1-benzyl-3-butylimidazolium acetate, 1,3-dioctylimidazolium acetate, 1,3-bis(1,1,3,3-tetramethylbutyl)imidazolium acetate, 1-ethyl-3-octylimidazolium acetate, 1-ethyl-3-(1,1,3,3-tetramethylbutyl)imidazolium acetate, 1-octyl-3-(2-ethylhexyl) imidazolium acetate, 1-1 (1,3,3-tetramethylbutyl)-3-(2-ethylhexyl) imidazolium acetate, 1-dodecyl-3-octylimidazolium acetate, 1-dodecyl-3-(1,1,3,3-tetramethylbutyl)imidazolium acetate, 1-octyl-3-octadecylimidazolium acetate, 1-(1,1,3,3-tetramethylbutyl)-3-octadecylimidazolium acetate, 1-benzyl-3-octylimidazolium acetate, 1-benzyl-3-(1,1,3,3-tetramethylbutyl)imidazolium acetate, 1,3-bis(2-ethylhexyl)imidazolium acetate, 1-ethyl-3-(2-ethylhexyl) imidazolium acetate, 1-(2-ethylhexyl)-3-dodecylimidazolium acetate, 1-(2-ethylhexyl)-3-octadecylimidazolium acetate, 1-benzyl-3-(2-ethylhexyl) imidazolium acetate, 1,3-didodecylimidazolium acetate, 1-dodecyl-3-octadecylimidazolium acetate, 1-benzyl-3-dodecylimidazolium acetate, 1,3-dioctadecylimidazolium acetate, 1-benzyl-3-octadecylimidazolium acetate, 1,3-dibenzylimidazolium acetate; 1,3-dimethylimidazolium 2-ethylhexanoate, 1-ethyl-3-methylimidazolium 2-ethylhexanoate, 1-butyl-3-methylimidazolium 2-ethylhexanoate, 1-methyl-3-octylimidazolium 2-ethylhexanoate, 1-methyl-3-(1,1,3,3-tetramethylbutyl)imidazolium 2-ethylhexanoate, 1-methyl-3-(2-ethylhexyl) imidazolium 2-ethylhexanoate, 1-dodecyl-3-methylimidazolium 2-ethylhexanoate, 1-methyl-3-octadecylimidazolium 2-ethylhexanoate, 1-benzyl-3-methylimidazolium 2-ethylhexanoate, 1,3-dibutylimidazolium 2-ethylhexanoate, 1-butyl-3-ethylimidazolium 2-ethylhexanoate, 1-butyl-3-octylimidazolium 2-ethylhexanoate, 1-butyl-3-(1,1,3,3-tetramethylbutyl)imidazolium 2-ethylhexanoate, 1-butyl-3-(2-ethylhexyl) imidazolium 2-ethylhexanoate, 1-butyl-3-dodecylimidazolium 2-ethylhexanoate, 1-butyl-3-octadecylimidazolium 2-ethylhexanoate, 1-benzyl-3-butylimidazolium 2-ethylhexanoate, 1,3-dioctylimidazolium 2-ethylhexanoate, 1,3-bis(1,1,3,3-tetramethylbutyl)imidazolium 2-ethylhexanoate, 1-ethyl-3-octylimidazolium 2-ethylhexanoate, 1-ethyl-3-(1,1,3,3-tetramethylbutyl)imidazolium 2-ethylhexanoate, 1-octyl-3-(2-ethylhexyl)imidazolium 2-ethylhexanoate, 1-(1,1,3,3-tetramethylbutyl)-3-(2-ethylhexyl) imidazolium 2-ethylhexanoate, 1-dodecyl-3-octylimidazolium 2-ethylhexanoate, 1-dodecyl-3-(1,1,3,3-tetramethylbutyl)imidazolium 2-ethylhexanoate, 1-octyl-3-octadecylimidazolium 2-ethylhexanoate, 1-(1,1,3,3-tetramethylbutyl)-3-octadecylimidazolium 2-ethylhexanoate, 1-benzyl-3-octylimidazolium 2-ethylhexanoate, 1-benzyl-3-(1,1,3,3-tetramethylbutyl)imidazolium 2-ethylhexanoate, 1,3-bis(2-ethylhexyl)imidazolium 2-ethylhexanoate, 1-ethyl-3-(2-ethylhexyl) imidazolium 2-ethylhexanoate, 1-(2-ethylhexyl)-3-dodecylimidazolium 2-ethylhexanoate, 1-(2-ethylhexyl)-3-octadecylimidazoliun 2-ethylhexanoate, 1-benzyl-3-(2-ethylhexyl) imidazolium 2-ethylhexanoate, 1,3-didodecylimidazolium 2-ethylhexanoate, 1-dodecyl-3-octadecylimidazolium 2-ethylhexanoate, 1-benzyl-3-dodecylimidazolium 2-ethylhexanoate, 1,3-dioctadecylimidazolium 2-ethylhexanoate, 1-benzyl-3-octadecylimidazolium 2-ethylhexanoate, 1,3-dibenzylimidazolium 2-ethylhexanoate; and 1,3-dimethylbenzimidazoliun formate, 1,3-dimethylbenzimidazolium acetate, and 1,3-dimethylbenzimidazolium 2-ethylhexanoate.

The imidazolium carboxylate (1) is preferably 1,3-dimethylimidazolium acetate, 1-ethyl-3-methylimidazolium acetate, 1-butyl-3-methylimidazolium acetate, 1-methyl-3-octylimidazolium acetate, 1-methyl-3-(1,1,3,3-tetramethylbutyl)imidazolium acetate, 1-methyl-3-(2-ethylhexyl) imidazolium acetate, 1-dodecyl-3-methylimidazolium acetate, 1-benzyl-3-methylimidazolium acetate, 1,3-dibutylimidazolium acetate, 1-butyl-3-ethylimidazolium acetate, 1-butyl-3-octylimidazolium acetate, 1-butyl-3-(1,1,3,3-tetramethylbutyl)imidazolium acetate, 1-butyl-3-(2-ethylhexyl) imidazolium acetate, 1-butyl-3-dodecylimidazolium acetate, 1-benzyl-3-butylimidazolium acetate, 1,3-dioctylimidazolium acetate, 1,3-bis(1,1,3,3-tetramethylbutyl)imidazolium acetate, 1-ethyl-3-octylimidazolium acetate, 1-ethyl-3-(1,1,3,3-tetramethylbutyl)imidazolium acetate, 1-octyl-3-(2-ethylhexyl)imidazolium acetate, 1-(1,1,3,3-tetramethylbutyl)-3-(2-ethylhexyl) imidazolium acetate, 1-dodecyl-3-octylimidazolium acetate, 1-dodecyl-3-(1,1,3,3-tetramethylbutyl)imidazolium acetate, 1-benzyl-3-octylimidazolium acetate, 1-benzyl-3-(1,1,3,3-tetramethylbutyl)imidazolium acetate, 1,3-bis(2-ethylhexyl) imidazolium acetate, I-ethyl-3-(2-ethylhexyl, imidazolium acetate, 1-(2-ethylhexyl)-3-dodecylimidazolium acetate, 1-benzyl-3-(2-ethylhexyl)imidazolium acetate, 1,3-didodecylimidazolium acetate, 1-benzyl-3-dodecylimidazolium acetate, or 1,3-dibenzylimidazolium acetate.

As the dicarbonyl compound (16), an aqueous solution or an alcohol solution, such as methanol or butanol, may be used as it is.

The amounts of the amine compound (17x) and amine compound (17y) (the amine compound (17x) and amine compound (17y) are hereinafter correctively referred to as “the amine compound (17)”) for use are usually such that the amount of the amine compound (17) is 0.1 to 10 mol, and preferably 0.5 to 3 mol, per mole of the dicarbonyl compound (16), 2 mol of the amine compound (17) is reacted per mole of the dicarbonyl compound (16) to form 1 mol of the imidazolium carboxylate (20-1); however, for example, if the amount of the amine compound (17) is less than 2 mol, the dicarbonyl compound (16) (raw material) and a polymer of the dicarbonyl compound (16) will be present in addition to the target imidazolium carboxylate (6-1). Further, if more than 2 mol of the amine compound (17) is used per mole of the dicarbonyl compound (16), an excessive amount of the amine compound (16) will be present in addition to the target imidazolium carboxylate (20-1). The imidazolium carboxylate (20-1) in which compounds other than an imidazolium cation are present together can also be used as a raw material for step 3.

The ratio of the amine compound (17x) to the amine compound (17y) (amine compound (17x):amine compound (17y)) is not particularly limited, and is within the range of 0:100 to 100:0. When the amine compound (17x):amine compound (17y) is 0:100, or the amine compound (17x):amine compound (17y) is 100:0, then R⁹=R¹⁰.

When formaldehyde is used as the aldehyde (18), an aqueous solution or an alcohol solution, such as methanol or butanol, may be used as it is. The amount of the aldehyde (18) for use is usually 0.1 to 10 mol, and preferably 0.5 to 5.0 mol, per mole of the dicarbonyl compound (16).

The amount of the monovalent aliphatic carboxylic acid (19), such as acetic acid, for use is usually 0.1 to 10 mol, preferably 0.5 to 2 mol, and even more preferably 1 to 1.5 mol, per mole of the dicarbonyl compound (16).

The optimal reaction temperature varies depending on the raw materials, solvents, etc. used, and is usually −10° C. or more, and preferably 0° C. to 100° C. The reaction time is usually 0.1 to 48 hours, and preferably 0.5 to 12 hours.

A solvent may or may not be used. When a solvent is used, the solvent used is not particularly limited, as long as it does not affect the reaction. Specific examples of solvents include aromatic hydrocarbons, such as toluene, benzene, and xylene; aliphatic or alicyclic hydrocarbons, such as methylcyclohexane, cyclohexane, hexane, heptane, and octane; halogenated hydrocarbons, such as dichloromethane, chloroform, carbon tetrachloride, and 1,2-dichloroethane; ethers, such as diethyl ether, tetrahydrofuran, and 1,4-dioxane; lower alcohols, such as methanol and ethanol; N,N-dimethylformamide, acetonitrile, water, and the like. Preferred among these are aromatic hydrocarbons, lower alcohols, and water; and particularly preferred are toluene and water. The solvents may be a mixture of two or more different types of solvents, if necessary.

The amount of the solvent for use is usually 50 parts by mass or less, and preferably 0.1 to 10 parts by mass, per part by mass of the dicarbonyl compound (16).

The reaction may be performed, if necessary, in an inert gas atmosphere, such as nitrogen, argon, or helium, which do not affect the reaction.

After completion of the reaction, the imidazolium carboxylate (20-1) can be obtained, for example, by removing impurities (e.g., unreacted raw materials) by washing with an organic solvent, or concentrating the reaction liquid, and may be purified by recrystallization, etc., if necessary.

Blocked Isocyanate Composition Comprising Blocked Isocyanate Compound and Onium Salt Represented by Formula (1)

The blocked isocyanate composition of the present invention comprises an onium salt represented by formula (1) and a blocked isocyanate compound.

Examples of the blocked isocyanate compound include compounds obtained by reacting isocyanates and blocking agents to block the isocyanate groups in the isocyanates with the blocking agents. The blocked isocyanate compounds may be used singly or as a mixture of two or more.

The isocyanate that constitutes the blocked isocyanate compound is not particularly limited, as long as it is a compound having two or more isocyanate groups. Examples of isocyanates include the following:
(i) aliphatic isocyanates,
(ii) alicyclic isocyanates,
(iii) aromatic isocyarates,
(iv) aromatic aliphatic isocyanates, and
(v) modified isocyanates formed from at least one member selected from the group consisting of aliphatic isocyanates, alicyclic isocyanates, aromatic isocyanates, and aromatic aliphatic isocyanates.

Preferred among these are the aliphatic isocyanates (i), alicyclic isocyanates (ii), and modified isocyanates (v) formed from at least one member selected from the group consisting of aliphatic isocyanates, alicyclic isocyanates, aromatic isocyanates, and aromatic aliphatic isocyanates.

These isocyanates may be used singly or as a mixture of two or more.

Examples of aliphatic isocyanates include 1,4-tetramethylene diisocyanate, 1,6-hexamethylene diisocyanate, 2,2,4-trimethylhexamethylene diisocyanate, 2,4,4-trimethylhexamethylene diisocyanate, lysine diisocyanate, dimer acid diisocyaniate, and the like.

Examples of alicyclic isocyanates include 1,3-bis(isocyanatomethyl)cyclohexane, 1,4-bis(isocyanatomethyl)cyclohexane, 3-isocyanatomethyl-3,3,5-trimethylcyclohexane (isophorone diisocyanate (IPDI)), bis-(4-isocyanatocyclohexyl)methane, norbornane diisocyanate, and the like.

Examples of aromatic isocyanates include 2,4′-diphenylmethane diisocyanate, 4,4′-diphenylmethane diisocyanate, crude diphenylmethane diisocyanate, 1,4-phenylene diisocyanate, 2,4-tolylene diisocyanate, 2,6-tolylene diisocyanate, 3,3′-dimethyl-4,4′-diisocyanatobiphenyl, 3,3′-dimethyl-4,4′-diisocyanatodiphenylmethane, 1,5-naphthylene diisocyanate, and the like.

Examples of aromatic aliphatic isocyanates include 1,3-xylylene diisocyanate, 1,4-xylylene diisocyanate, α,α,α′,α′-tetramethylxylylene diisocyanate, and the like.

Examples of modified isocyanates include isocyanate-terminated compounds obtained by the reaction of the above isocyanate compounds with compounds having an active hydrogen group, and reaction products of the isocyanate compounds and/or the isocyanate-terminated compounds (e.g., adduct-type isocyanates, and modified isocyanates obtained by allophanatization reaction, carbodiimidization reaction, uretodionization reaction, isocyanuration reaction, uretoniminization reaction, biuretization reaction, or the like); and preferably adduct-type isocyanates, isocyanates modified by isocyanuration reaction, and isocyanates modified by biuretization reaction (isocyanates having a biuret bond).

An isocyanate having a biuret bond is obtained by reacting a so-called biuretizing agent, such as water, tert-butanol, or urea, with an isocyanate at a molar ratio of the biuretizing agent/isocyanate groups in the isocyanate of about 1/2 to about 1/100, followed by purification by removing the unreacted isocyanate. An isocyanate having an isocyanurate bond is obtained, for example, by performing the cyclic trimerization reaction using a catalyst etc., stopping the reaction when the conversion rate reaches about 5 to about 80 mass %, and removing the unreacted isocyanate for purification. In this case, a mono- to hexavalent alcohol compound can be used in combination.

Examples of isocyanates having a biuret bond include a biuret modified product of 1,6-hexamethylene diisocyanate (HDI), a biuret modified product of isophorone diisocyanate (IPDI), and a biuret modified product of toluene diisocyanate (TDI) shown below. Commercial products include Desmodur N75, Desmodur N100, and Desmodur N3200 (all produced by Sumika Covestro Urethane Co., Ltd.); Duranate 24A-100, Duranate 22A-75P, and Duranate 21S-75E (all produced by Asahi Kasei Corporation); and the like.

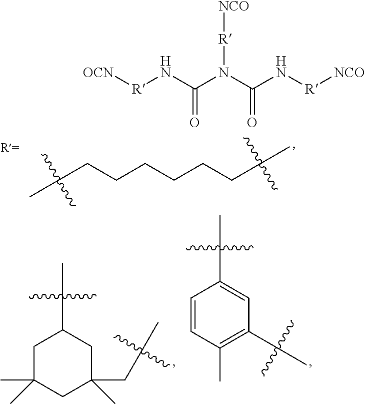

An isocyanate having an isocyanurate bond is obtained, for example, by performing an isocyanuration reaction using a catalyst etc., stopping the reaction when the conversion rate reaches about 5 to about 80 mass %, and removing the unreacted isocyanate for purification. In this case, a mono- to hexavalent alcohol compound can be used in combination.

The catalyst for the above isocyanuration reaction is generally preferably a basic catalyst.

Examples of the catalyst include the following:
(1) hydroxides of tetraalkylanmonium, such as tetramethylammonium, tetraethylammonium, and trimethylbenzylammonium; and organic weak acid salts, such as acetic acid and capric acid;
(2) hydroxides of hydroxyalkylammonium, such as trimethylhydroxypropylammonium, trimethylhydroxyethylammonium, triethylhydroxypropylammonium, and triethylhrdroxyethylammionium; and organic weak acid salts, such as acetic acid and capric acid;
(3) metal salts of alkyl carboxylic acids with, for example, tin, zinc, and lead;
(4) metal alcoholates of sodium, potassium, etc.;
(5) aminosilyl group-containing compounds, such as hexamethyldisilazane;
(6) Mannich bases;
(7) combination of tertiary amines and epoxy compounds; and
(8) phosphorus compounds, such as tributylphosphine.

These can be used in combination of two or more.

If the catalyst may adversely affect paints or coating film properties, the catalyst may be neutralized with an acidic compound. Examples of acidic compounds include inorganic acids, such as hydrochloric acid, phosphorous acid, and phosphoric acid; sulfonic acids or derivatives thereof, such as methanesulfonic acid, p-tolueneoulfonic acid, p-toluenesulfcnic acid methyl ester, and p-toluenesulfonic acid ethyl ester; ethyl phosphate, diethyl phosphate, isopropyl phosphate, diisopropyl phosphate, butyl phosphate, dibutyl phosphate, 2-ethylhexyl phosphate, di(2-ethylhexyl)phosphate, isodecyl phosphate, diisodecyl phosphate, oleyl acid phosphate, tetracosyl acid phosphate, ethyl glycol acid phosphate, butyl pyrophosphate, butyl phosphite, and the like. These may be used in combination of two or more.

Examples of isocyanates having an isocyanurate bond include isocyanurate-modified HDI, isocyanurate-modified IPDI, and isocyanurate-modified TDI shown below. Commercial products include Sumidur N3300, Desmodur 3900, Desmodur Z4470BA, Desmodur XP2763, Desmodur I:1351BA, and Desmodur HIBA (all produced by Sumika Covestro Urethane Co., Ltd.); Duranate TPA-100, Duranate MFA-75B, Duranate TUL-100, and Duranate TSA-100 (all produced by Asahi Kasei Corporation); and the like.

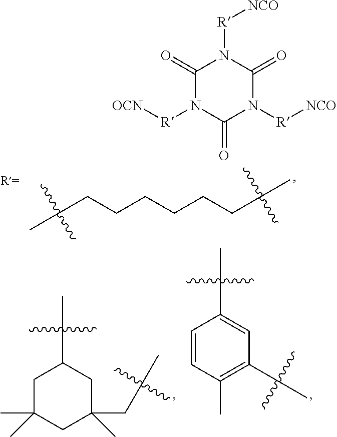

An isocyanate having a urethane bond is obtained, for example, by reacting a di- to hexavalent alcohol compound, such as trimethylolpropane (hereinafter referred to as TMP), with a diisocyanate at a molar ratio of hydroxyl groups in the alcohol compound/isocyanate groups in the isocyanate of about 1/2 to about 1/100, and then removing the unreacted isocyanate for purification. Removal of the unreacted isocyanate for purification is not necessarily required.

Examples of isocyanates having a urethane bond include a reaction product of HDI and TMP, a reaction product of IPDI and TMP, and a reaction product of TDI and TMP shown below. Commercial products include Sumidur HT, Desmodur L75(C), Desmodur Ultra L75, and Desmodur L67 BA (all produced by Sumika Covestro Urethane Co., Ltd.); Duranate P301-75E, Duranate AE700-100, Duranate E402-80B, and Duranate E405-70B (all produced by Asahi Kasei Corporation); and the like.

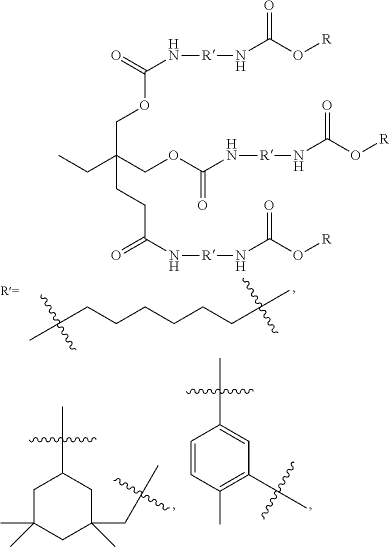

Examples of known blocking agents for isocyanates in which some isocyanate groups of the above isocyanates or modified isocyanates are blocked with known blocking agents include phenols, such as phenol, thiophenol, methylthiophenol, xylenol, cresol, resorcinol, nitrophenol, and chlorophenol; oximes, such as acetone oxime, methyl ethyl ketone oxime, and cyclohexanone oxime; alcohols, such as methanol, ethanol, n-propyl alcohol, isopropyl alcohol, n-butyl alcohol, isobutyl alcohol, t-butyl alcohol, t-pentanol, ethylene glycol monoethyl ether, ethylene glycol monobutyl ether, diethylene glycol moroethyl ether, propylene glycol monomethyl ether, and benzyl alcohol; pyrazoles, such as 3,5-dimethyl pyrazole and 1,2-pyrazole; triazoles, such as 1,2,4-triazole; halogen-substituted alcohols, such as ethylene chlorohydrin and 1,3-dichloro-2-propanol; lactams, such as ε-caprolactam, δ-valerolactam, γ-butyrolactam, and β-propyllactam; active methylene compounds, such as methyl acetoacetate, ethyl acetoacetate, acetylacetone, methyl malonate, and ethyl malonate; and the like. Other examples include amines, imides, mercaptans, imines, ureas, diaryls, and the like.

Examples of blocking agents include alcohol compounds, phenol compounds, amine compounds, lactam compounds, oxime compounds, ketoenol compounds, active methylene compounds, pyrazole compounds, triazole compounds, imide compounds, mercaptan compounds, imine compounds, urea compounds, and diaryl compounds; preferably alcohol compounds, lactam compounds, oxime compounds, and pyrazole compounds; preferably alcohol compounds because when they are combined with the onium salt represented by formula (1), the blocking agent can be dissociated in a short period of time particularly even at low temperatures of less than 100° C.; and particularly preferably fluorinated alcohol compounds.

Examples of alcohol compounds include methanol, ethanol, n-propyl alcohol, isopropyl alcohol, n-butyl alcohol, isobutyl alcohol, t-butyl alcohol, t-pentanol, ethylene glycol monoethyl ether, ethylene glycol monobutyl ether, diethylene glycol monoethyl ether, propylene glycol monomethyl ether, benzyl alcohol, and the like. Alcohol compounds include fluorinated alcohol compounds, such as 2,2,2-trifluoroethanol and 1,1,1,3,3,3-hexafluoro-2-propanol.

The alcohol compound is preferably a fluorinated alcohol compound, and particularly preferably 2,2,2-trifluoroethanol.

Examples of phenol compounds include phenol, thiophenol, methylthiophenol, xylenol, cresol, resorcinol, nitrophenol, chlorophenol, 2-hydroxypyridine, and the like.

Examples of amine compounds include diisopropylamine and the like.

Examples of lactam compounds include ε-caprolactam, δ-valerolactam, γ-butyrolactam, and the like; and preferably ε-caprolactam.

Specific examples of oxime compounds include formaldehyde oxime, acetaldehyde oxime, acetone oxime, methyl ethyl ketoxime, methyl isobutyl ketoxime, and the like; and preferably methyl ethyl ketoxime.

Examples of pyrazole compounds include 1,2-pyrazole, 3,5-dimethylpyrazole, and the like. Examples of triazole compounds include 1,2,4-triazole and the like, and preferably 3,5-dimethylpyrazole.

Examples of active methylene compounds include methyl acetoacetate, ethyl acetoacetate, acetylacetone, methyl malonate, ethyl malonate, and the like.

In the blocked isocyanate composition of the present invention, known catalysts for polyurethane production, additives, pigments, solvents, and the like that are commonly used in this technical field can be used, if necessary.

Known catalysts for polyurethane production are not particularly limited. Examples include tin compounds, such as dibutyltin dilaurate, dibutyltin di-2-ethylhexanate, dioctyltin dilaurate, dibutyltin diacetate, dibutyltin dioxide, dioctyltin dioxide, tin acetylacetonate, tin acetate, tin octylate, and tin laurate; bismuth compounds, such as bismuth octylate, bismuth naphthenate, and bismuth acetylacetonate; titanium compounds, such as tetra-n-butyl titanate, tetraisopropyl titanate, and titanium terephthalate; tertiary amine compounds, such as triethylamine, N,N,N′,N′-tetramethylethylenediamine, N,N,N′,N′-tetramethylpropylenediamine, N,N,N′,N″,N″-pentamethyldiethylenetriamine, N,N,N′,N″,N″-pentamethyldipropylenetriamine, N,N,N,N′-tetramethylguanidine, 1,3,5-tris(N,N-dimethylaminopropyl)hexahydro-S-triazine, 1,4-diazabicyclo[2.2.2]octane (DABCO), 1,8-diazabicyclo[5.4.0]undecene-7, triethylenediamine, N,N,N′,N′-tetramethylhexamethylenediamine, N-methyl-N′-(2-dimethylaminoethyl)piperazine, N,N′-dimethylpiperazine, dimethylcyclohexylamine, N-methylmorpholine, N-ethylrorpholine, bis(2-dimethylaminoethyl)ether, 1-methylimidazole, 1,2-dimethylimidazole, 1-isobutyl-2-methylimidazole, and 1-dimethylaminopropylimidazole; and quaternary ammonium salt compounds, such as tetraalkylammonium halides (e.g., tetramethylammonium chloride), tetraalkylammonium hydroxides (e.g., tetramethylarmmnium hydroxide salts), tetraalkylammonium organic acid salts (e.g., tetramethylammonium-2-ethylhexanoate, 2-hydroxypropyl trimethylamnonium formate, and 2-hydroxypropyl trimethylamonium-2-ethylhexanoate).

Additives are not particularly limited. Examples include hindered amine-based, benzotriazole-based, and benzophenone-based N absorbers; perchlorate-based and hydroxylamine-based coloration inhibitors; hindered phenol-based, phosphorus-based, sulfur-based, and hydrazide-based antioxidants; tin-based, zinc-based, and amine-based urethanization catalysts; leveling agents, rheology control agents, pigment dispersants, and the like.

Pigments are not particularly limited. Examples include organic pigments, such as quinacridone-based, azo-based, and phthalocyanine-based pigments; inorganic pigments, such as titanium oxide, barium sulfate, calcium carbonate, and silica; and other pigments, such as carbon-based pigments, metal foil pigments, and rust-preventive pigments.

Solvents are not particularly limited. Examples include hydrocarbons, such as benzene, toluene, xylene, cyclohexane, mineral spirit, and naphtha; ketones, such as acetone, methyl ethyl ketone, and methyl isobutyl ketone; esters, such as ethyl acetate, butyl acetate, and cellosolve acetate; alcohols, such as methanol, ethanol, 2-propanol, butanol, 2-methoxyethanol, 2-ethoxyethanol, and 2-butoxyethanol; polyhydric alcohols, such as ethylene glycol, propylene glycol, diethylene glycol, polyethylene glycol, and glycerol; water; and the like. These solvents may be used singly or in combination of two or more.

The thermosetting resin composition of the present invention is explained.

The thermosetting resin composition of the present invention comprises the blocked isocyanate composition of the present invention and a compound having an isocyanate-reactive group.

Examples of the compound having an isocyanate-reactive group include compounds having two or more active hydrogen groups, such as polyols, polyamines, and alkanolamines. These compounds having an isocyanate-reactive group may be a mixture of two or more.

In the present invention, polyols are compounds having two or more hydroxyl groups. Examples include polyester polyols, polyether polyols, acrylic polyols, polyolefin polyols, fluorine polyols, and the like. Preferred polyols among these are acrylic polyols in terms of weather resistance, chemical resistance, and hardness. Alternatively, polyols preferred in terms of mechanical strength and oil resistance are polyester polyols. These polyols may be a mixture of two or more.

Examples of polyether polyols include active hydrogen compounds, such as aliphatic amine polyols, aromatic amine polyols, Mannich polyols, polyhydric alcohols, polyhydric phenols, and bisphenols; compounds obtained by adding alkylene oxides to these active hydrogen compounds; and the like. These polyether polyols may be a mixture of two or more.

Examples of aliphatic amine polyols include alkylenediamine-based polyols and alkanolamine-based polyols. These polyol compounds are polyfunctional polyol compounds having terminal hydroxyl groups obtained by the ring-opening addition of at least one cyclic ether, such as ethylene oxide or propylene oxide, using alkylenediamine or alkanolamine as an initiator. As the alkylenediamine, known compounds can be used without limitation. Specifically, C₂₋₆ alkylenediamines, such as ethylenediamine, propylenediamine, butylenediamine, hexamethylenediamine, and neopentyldiamine, are preferably used. These aliphatic amine polyols may be a mixture of two or more.

Aromatic amine polyols are polyfunctional polyether polyol compounds having terminal hydroxyl groups obtained by the ring-opening addition of at least one cyclic ether, such as ethylene oxide or propylene oxide, using an aromatic diamine as an initiator. As the initiator, a known aromatic diamine can be used without limitation. Specific examples include 2,4-toluenediamine, 2,6-toluenediamine, diethyltoluenediamine, 4,4′-diaminodiphenylmethane, p-phenylenediamine, o-phenylenediamine, naphthalenediamine, and the like. Among these, toluenediamine (2,4-toluenediamine, 2,6-toluenediamine, or a mixture thereof) is particularly preferably used. These aromatic amine polyols may be a mixture of two or more.

Mannich polyols are active hydrogen compounds obtained by the Mannich reaction of phenol and/or an alkyl-substituted derivative thereof, formaldehyde, and alkanolamine, or polyol compounds obtained by the ring-opening addition polymerization of the active hydrogen compounds with at least one of ethylene oxide and propylene oxide. These Mannich polyols may be a mixture of two or more.

Examples of polyhydric alcohols include dihydric alcohols (e.g., ethylene glycol, propylene glycol, 1,4-butanediol, 1,6-hexanediol, diethylene glycol, triethylene glycol, dipropylene glycol, and neopentyl glycol), trihydric or higher alcohols (e.g., glycerol, trimethylolpropane, pentaerythritol, methylglucoside, sorbitol, and sucrose), and the like. These polyhydric alcohols may be a mixture of two or more.

Examples of polyhydric phenols include pyrogallol, hydroquinone, and the like. These polyhydric phenols may be a mixture of two or more.

Examples of bisphenols include bisphenol A, bisphenol S, bisphenol F, low-condensates of phenols and formaldehyde, and the like. These bisphenols may be a mixture of two or more.

Polyester polyols can be obtained, for example, by the condensation reaction of a single dibasic acid or a mixture of two or more dibasic acids with a single polyhydric alcohol or a mixture of two or more polyhydric alcohols.

Examples of dibasic acids include carboxylic acids, such as succinic acid, adipic acid, dimer acid, maleic anhydride, phthalic anhydride, isophthalic acid, terephthalic acid, and 1,4-cyclohexanedicarboxylic acid; and the like.

Examples of polyhydric alcohols include ethylene glycol, propylene glycol, diethylene glycol, 1,4-butanediol, neopentyl glycol, 1,6-hexanediol, trimethylpentanediol, cyclohexanediol, trimethylolpropane, glycerol, pentaerythritol, 2-methylolpropanediol, ethoxylated trimethylolpropane, and the like.

As a specific method for producing polyester polyols, for example, the condensation reaction can be carried out by mixing the above components, and heating the mixture at about 160 to 220° C. Alternatively, for example, polycaprolactones obtained by the ring-opening polymerization of lactones, such as caprolactone, with polyhydric alcohols can also be used as polyester polyols.

These polyester polyols can be modified by using, for example, aromatic diisocyanates, aliphatic diisocyanates, alicyclic diisocyanates, and isocyanates obtained from them. Among these, in terms of weather resistance, yellowing resistance, etc., polyester polyols are preferably modified by using aliphatic diisocyanates, alicyclic diisocyanates, and isocyanates obtained from them.

When the thermosetting resin composition of the present embodiment is used as an aqueous-based paint, some carboxylic acids derived from the dibasic acid etc. in the polyester polyol can be allowed to remain and neutralized with a base, such as amine or ammonia, thereby forming the polyester polyol into a water-soluble or water-dispersible resin.

Polyether polyols can be obtained, for example, by any of the following methods (1) to (3).

(1) A method of performing random or block addition of an alkylene oxide alone or a mixture of alkylene oxides to a polyhydroxy compound alone or a mixture of polyhydroxy compounds using a catalyst to obtain polyether polyols.

Examples of catalysts include hydroxides (of lithium, sodium, potassium, etc.), strong base catalysts (alcoholates, alkylamines, etc.), composite metal cyanide compound complexes (metal porphyrins, zinc hexacyanocobaltate complexes, etc.), and the like.

Examples of alkylene oxides include ethylene oxide, propylene oxide, butylene oxide, cyclohexene oxide, styrene oxide, and the like.

(2) A method of reacting polyamine compounds with alkylene oxides to obtain polyethers polyols.

Examples of polyamine compounds include ethylene diamines and the like.

Examples of alkylene oxides include those mentioned in (1).

(3) A method of polymerizing acrylamide etc. using the polyether polyols obtained in (1) or (2) as media to obtain so-called polymer polyols.

Examples of polyhydroxy compounds include the following (i) to (vi).
(i) diglycol, ditrimethylolpropane, pentaerythritol, dipentaerythritol, etc.
(ii) sugar alcohol compounds, such as erythritol, D-threitol, L-arabinitol, ribitol, xylitol, sorbitol, mannitol, galactitol, and rhamnitol
(iii) monosaccharides, such as arabinose, ribose, xylose, glucose, mannose, galactose, fructose, sorbose, rhamnose, fucose, and ribodesose
(iv) disaccharides, such as trehalose, sucrose, maltose, cellobiose, gentiobiose, lactose, and melibiose
(v) trisaccharides, such as raffinose, gentianose, and melezitose
(vi) tetrasaccharides, such as stachyose

Acrylic polyols can be obtained, for example, by polymerizing polymerizable monomers having one or more active hydrogens per molecule, or by copolymerizing polymerizable monomers having one or more active hydrogens per molecule with other monomers copolymerizable with the polymerizable monomers, if necessary.

Examples of polymerizable monomers having one or more active hydrogens per molecule include the following (i) to (vi). These may be used singly or in combination of two or more.
(i) acrylic acid esters having active hydrogen, such as 2-hydroxyethyl acrylate, 2-hydroxypropyl acrylate, and 2-hydroxybutyl acrylate
(ii) methacrylic acid esters having active hydrogen, such as 2-hydroxyethyl methacrylate, 2-hydroxypropyl methacrylate, 2-hydroxybutyl methacrylate, 3-hydroxypropyl methacrylate, and 4-hydroxybutyl methacrylate
(iii) (meth)acrylic acid esters having polyvalent active hydrogen, such as (meth)acrylic acid monoesters of triols, such as glycerol and trimethylolpropane
(iv) monoethers of polyether polyols (e.g., polyethylene glycol, polypropylene glycol, and polybutylene glycol) with the above (meth)acrylic acid esters having active hydrogen
v) adducts of glycidyl (meth)aczylate with monobasic acids (e.g., acetic acid, propionic acid, and p-tert-butyl benzoic acid)
(vi) adducts obtained by the ring-opening polymerization of lactones (e.g., ε-caprolactam and γ-valerolactone) with the active hydrogen of the above (meta)acrylic acid esters having active hydrogen

Examples of monomers copolymerizable with the above polymerizable monomers include the following (i) to (iv). These may be used singly or in combination of two or more.
(i) (meth)acrylic acid esters, such as methyl acrylate, ethyl acrylate, isopropyl acrylate, n-butyl acrylate, 2-ethylhexyl acrylate, methyl methacrylate, ethyl methacrylate, isopropyl methacrylate, n-butyl methacrylate, isobutyl methacrylate, n-hexyl methacrylate, cyclohexyl methacrylate, lauryl methacrylate, and glycidyl methacrylate
(ii) unsaturated carboxylic acids, such as acrylic acid, methacrylic acid, maleic acid, and itaconic acid; and unsaturated amides, such as acrylamide, N-methylolacrylamide, and diacetoneacrylamide
(iii) vinyl monomers having a hydrolyzable silyl group, such as vinyl trimethoxysilane, vinyl methyl dimethoxysilane, and γ-(meth)acrylopropyltrimethoxysilane
(iv) other polymerizable monomers, such as styrene, vinyl toluene, vinyl acetate, acrylonitrile, and dibutyl fumarate

As a specific method for producing acrylic polyols, for example, the above monomer components are subjected to solution polymerization in the presence of a known radical polymerization initiator, such as a peroxide or an azo compound, optionally followed by dilution with an organic solvent etc., thereby obtaining acrylic polyols.

When the thermosetting resin composition of the present embodiment is used as an aqueous-based paint, aqueous-based acrylic polyols can be produced by solution polymerization of the above monomer components, and conversion to an aqueous layer, or by using a known method, such as emulsion polymerization. In that case, the acidic portions of carboxylic acid-containing monomers and sulfonic acid-containing monomers, such as acrylic acid and methacrylic acid, can be neutralized with amine or ammonia to make acrylic polyols water-soluble or water-dispersible.

Examples of polyolefin polyols include polybutadiene having two or more hydroxyl groups, hydrogenated polybutadiene having two or more hydroxyl groups, hydrogenated polyisoprene having two or more hydroxyl groups, and the like.

In the polyolefin polyol, the number of hydroxyl groups is preferably three because higher coating film strength can be obtained.

In the present specification, “fluorine polyols” refer to polyols containing fluorine in the molecule. Specific examples of fluorine polyols include the copolymers of fluoroolefin, cyclovinyl ether, hydroxyalkyl vinyl ether, and vinyl monocarboxylate disclosed in JPS57-34107A, JPS61-275311A, etc.

The lower limit of the hydroxyl value of the polyol is preferably 10 mgKOH/g or more, more preferably 20 mgKOH/g or more, and even more preferably 30 mgKOH/g or more.

On the other hand, the upper limit of the hydroxyl value of the polyol is not particularly limited, and may be, for example, 200 mgKOH/g or less.

Specifically, the hydroxyl value of the polyol is preferably 10 mgKOH/g or more and 200 mgKOH/g or less, more preferably 20 mgKOH/g or more and 200 mgKOH/g or less, and even more preferably 30 mgKOH/g or more and 200 mgKOH/g or less.

Further, the acid value of the polyol is preferably 0 mgKOH/g or more and 30 mgKOH/g or less.

The hydroxyl value and acid value can be measured according to JIS K1557.

The molar equivalent ratio (NCO/OH) of isocyanate groups in the blocked isocyanate composition to hydroxyl groups in the polyol is preferably 0.2 or more and 5.0 or less, more preferably 0.4 or more and 3.0 or less, and even more preferably 0.5 or more and 2.0 or less.

Usable polyamines are those having two or more primary amino groups or secondary amino groups per molecule. Preferred among these are those having three or more such amino groups per molecule.

Specific examples of polyamines include diamines, such as ethylenediamine, propylenediamine, butylenediamine, triethylenediamine, hexamethylenediamine, 4,4′-diaminodicyclohexylmethane, piperazine, 2-methylpiperazine, and isophoronediamine; chain polyamines having three or more amino groups, such as bishexamethylenetriamine, diethylenetriamine, triethylenetetramine, tetraethylenepentamine, pentamethylenehexamine, and tetrapropylenepentamine; and cyclic polyamines, such as 1,4,7,10,13,16-hexaazacyclooctadecane, 1,4,7,10-tetraazacyclodecane, 1,4,8,12-tetraazacyclopentadecane, and 1,4,8,11-tetraazacyclotetradecane.

Alkanolamines refer to compounds having an amino group and a hydroxyl group per molecule. Examples of alkanolamines include monoethanolamine, diethanolamine, aminoethylethanolamine, N-(2-hydroxypropyl)ethylenediamine, mono-, di-(n- or iso-)propanolamine, ethylene glycol-bis-propylamine, neopentanolamine, methylethanclamine, and the like.

The thermosetting resin composition of the present embodiment may contain, if necessary, melamine-based curing agents, such as complete alkyl type, methylol type, and alkylamino group type alkyl.

The thermosetting resin composition of the present embodiment may contain an organic solvent.

Further, the compound having an isocyanate-reactive group and the blocked isocyanate composition described above may contain an organic solvent.

Preferable organic solvents are those that are compatible with the blocked isocyanate composition.

Specific examples of organic solvents include hydrocarbons, such as benzene, toluene, xylene, cyclohexane, mineral spirit, and naphtha; ketones, such as acetone, methyl ethyl ketone, and methyl isobutyl ketone; esters, such as ethyl acetate, butyl acetate, and cellosolve acetate; alcohols, such as methanol, ethanol, 2-propanol, butanol, 2-methoxyethanol, 2-ethoxyethanol, and 2-butoxyethanol; polyhydric alcohols, such as ethylene glycol, propylene glycol, diethylene glycol, polyethylene glycol, and glycerol; water; and the like. These solvents may be used singly or in combination of two or more.

Further, the thermosetting resin composition of the present embodiment can be used as an aqueous thermosetting resin composition dissolved or dispersed in water. When the thermosetting resin composition of the present invention is used as an aqueous thermosetting resin composition, in order to improve the compatibility of the thermosetting resin composition, surfactants, solvents that tend to be miscible with water, etc. may be used for the blocked isocyanate composition of the present invention. Examples of surfactants include anionic surfactants, such as aliphatic soaps, rosin acid soaps, alkyl sulfonates, dialkylaryl sulfonates, alkyl sulfosuccinates, polyoxyethylene alkyl sulfates, and polyoxyethylene alkylaryl sulfates; and nonionic surfactants, such as polyoxyethylene alkyl ethers, polyoxyethylene alkyl aryl ethers, and polyoxyethylene oxypropylene block copolymers. Examples of solvents that tend to be miscible with water include diethylene glycol dimethyl ether, diethylene glycol diethyl ether, propylene glycol monomethyl ether acetate, propylene glycol monomethyl ether, propylene glycol dimethyl ether, dipropylene glycol dimethyl ether, isobutanol, butyl glycol, N-methylpyrrolidone, butyl diglycol, butyl diglycol acetate, and the like.

Preferred among the above solvents are diethylene glycol dimethyl ether, diethylene glycol diethyl ether, propylene glycol monomethyl ether, propylene glycol dimethyl ether, dipropylene glycol dimethyl ether, isobutanol, butyl glycol, N-methylpyrrolidone, and butyl diglycol; and more preferred are diethylene glycol dimethyl ether, diethylene glycol diethyl ether, propylene glycol dimethyl ether, and dipropylene glycol dimethyl ether. These solvents may be used singly or in combination of two or more. Ester solvents, such as ethyl acetate, n-butyl acetate, and cellosolve acetate, are not preferred because the solvents themselves may hydrolyze during storage.

In the thermosetting resin composition of the present invention, the mixing ratio of the blocked isocyanate composition and the compound having an isocyanate-reactive group is determined by the required physical properties, and is not particularly limited. The mixing ratio is generally within the following range: [the amount of effective isocyanate groups (mol) in the blocked isocyanate compound in the blocked isocyanate composition]/[the amount of active hydrogen groups (mol) in the compound having an isocyanate-reactive group]=0.2 to 5, and preferably 0.5 to 3. The effective isocyanate groups in the blocked isocyanate compound refer to isocyanate groups that are regenerated when the blocking agent is dissociated from the blocked isocyanate compound.

In the thermosetting resin composition of the present invention, known catalysts for polyurethane production, additives, pigments, and the like that are commonly used in this technical field can be used, if necessary. These may be used as a mixture with known blocked isocyanates.

Known catalysts for polyurethane production are not particularly limited. Examples include tin compounds, such as dibutyltin dilaurate, dibutyltin di-2-ethylhexanate, dioctyltin dilaurate, dibutyltin diacetate, dibutyltin dioxide, dioctyltin dioxide, tin acetylacetonate, tin acetate, tin octylate, and tin laurate; bismuth compounds, such as bismuth octylate, bismuth naphthenate, and bismuth acetylacetonate; titanium compounds, such as tetra-n-butyl titanate, tetraisopropyl titanate, and titanium terephthalate; tertiary amine compounds, such as triethylamine, N,N,N′,N′-tetramethylethylenediamine, N,N,N′,N′-tetramethylpropylenediamine, N,N,N′,N″,N″-pentamethyldiethylenetriamine, N,N,N′,N″,N″-pentamethyldipropylenetriamine, N,N,N′,N′-tetramethylguanidine, 1,3,5-tris(N,N-dimethylaminopropyl)hexahydro-S-triazine, 1,4-diazabicyclo[2.2.2]octane (DABCO), 1,8-diazabicyclo[5.4.0]undecene-7, triethylenediamine, N,N,N′,N′-tetramethylhexamethylenediamine, N-methyl-N′-(2-dimethylaminoethyl)piperazine, N,N′-dimethylpiperazine, dimethylcyclohexylamine, N-methylmorpholine, N-ethylmorpholine, bis(2-dimethylaminoethyl)ether, 1-methylimidazole, 1,2-dimethylimidazole, 1-isobutyl-2-methylimidazole, and 1-dimethylaminopropylimidazole; and quaternary ammonium salt compounds, such as tetraalkylammonium halides (e.g., tetramethylammonium chloride), tetraalkylanonium hydroxides (e.g., tetramethylammonium hydroxide salts), tetraalkylammonium organic acid salts (e.g., tetramethylannonium-2-ethylhexanoate, 2-hydroxypropyl trimethylammonium formate, and 2-hydroxypropyl trimethylammonium-2-ethylhexanoate).

Additives are not particularly limited. Examples include hindered amine-based, benzotriazole-based, and benzophenone-based UV absorbers; perchlorate-based and hydroxylamine-based coloration inhibitors; hindered phenol-based, phosphorus-based, sulfur-based, and hydrazide-based antioxidants; tin-based, zinc-based, and amine-based urethanization catalysts; leveling agents, antifoaming agents, rheology control agents, thixotropy-imparting agents, thickeners, light stabilizers, plasticizers, surfactants, coupling agents, flame retardants, rust inhibitors, fluorescent whitening agents, pigment dispersants, and the like that are commonly used in this technical field.

Pigments are not particularly limited. Examples include organic pigments, such as quinacridone-based, azo-based, and phthalocyanine-based pigments; inorganic pigments, such as titanium oxide, barium sulfate, calcium carbonate, and silica; and other pigments, such as carbon-based pigments, metal foil pigments, and rust-preventive pigments.

Examples of known blocked isocyanates include blocked isocyanates obtained by reacting isocyanates and known blocking agents. Examples of known blocking agents include phenol compounds, such as phenol, thiophenol, methylthiophenol, xylenol, cresol, resorcinol, nitrophenol, and chlorophenol; oxime compounds, such as acetone oxime, methyl ethyl ketone oxime, and cyclohexanone oxime; alcohol compounds, such as methanol, ethanol, n-propyl alcohol, isopropyl alcohol, n-butyl alcohol, isobutyl alcohol, t-butyl alcohol, t-pentanol, ethylene glycol monoethyl ether, ethylene glycol monobutyl ether, diethylene glycol monoethyl ether, propylene glycol monomethyl ether, and benzyl alcohol; pyrazole compounds, such as 3,5-dimethyl pyrazole and 1,2-pyrazole; triazole compounds, such as 1,2,4-triazole; halogen-substituted alcohol compounds, such as ethylene chlorohydrin and 1,3-dichloro-2-propanol; lactam compounds, such as ε-caprolactam, δ-valerolactam, γ-butyrolactam, and β-propyllactam; active methylene compounds, such as methyl acetoacetate, ethyl acetoacetate, acetylacetone, methyl malonate, and ethyl malonate; and the like. Other examples include amine compounds, imide compounds, mercaptan compounds, imine compounds, urea compounds, diaryl compounds, and the like.

The thermosetting resin composition of the present invention can be used as paints for automobiles, for buildings, for metal products such as steel furniture, for wooden products such as musical instruments, for mechanical vehicles such as construction machinery, for building materials such as sashes, and for electrical appliances such as office equipment; coating materials for artificial leather, rubber rolls, etc.; inks, adhesives, pressure-sensitive adhesives, sealing materials for electronic components, sealing materials for automobiles, buildings, etc., molding materials for 3D printers, and the like.

Next, the method for curing the thermosetting resin composition of the present invention is explained.

In the method of the present invention, the thermosetting resin composition, which is a mixture of the blocked isocyanate composition and the compound having an isocyanate-reactive group described above, is heated.

The reaction temperature varies depending on the blocked isocyanate compound and the onium salt (1) in the blocked isocyanate composition used, but is generally about 60 to 250° C., and preferably about 80 to 200° C. The reaction time is about 30 seconds to 5 hours, and preferably about 1 minute to 60 minutes.

The cured product of the present invention can be produced through the above method for curing the thermosetting resin composition of the present invention.

### EXAMPLES

The present invention is described in more detail below with reference to Production Examples and Examples; however, the present invention is not limited to these Examples.

### (1) Conditions of Infrared Spectroscopy (IR Analysis)

Device: FT/IR-6600, produced by JASCO Corporation
Measurement method: total reflection measurement method (crystal: germanium)
Cumulative number: 16

### (2)¹H-NMR Analysis Conditions

Device: AV400 produced by Bruker Corporation
Frequency: 400 MHz

### (3) Measurement Conditions of Curing Temperature and Time

Device: Madoka automatic curing time measuring device produced by Cyber Co., Ltd.
Stirring rod: Model number 3JC-5060W
Stirring rate: rotation 100 rpm, revolutions 25 rpm

### (4) Calculation of Solids Content

About 1.5 g of a sample was heated at 110° C. for 3 hours, and the solids content (%) in the sample was calculated from the mass before and after heating.

### (5) Formulation of Thermosetting Resin Composition

A blocked isocyanate compound, a compound having an isocyanate-reactive group, and an amidate compound were added such that effective NCO group (mol):hydroxyl group (mol):amidate group (mol)=1.00:0.95:0.05, and methyl isobutyl ketone was added such that solids content in the blocked isocyanate compound (g):solvent (g)=1.0:1.0. The solvent referred to here includes the solvent used for diluting the blocked isocyanate compound. The effective NCO group (mol) and hydroxyl group (mol) were calculated according to the following formulas.

Effective NCO group (mol)=amount of the blocked isocyanate used (g)/effective NCO group content (t) in the blocked isocyanate/4.202

Hydroxyl group (mol)=amount of the polyol used (g)×hydroxyl value of the polyol (mgKOH/g)/56.1

### Production Example 1: Synthesis of 1,3-di-tert-butylimidazolium hydrogen carbonate ([tBurI] [C])

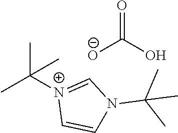

In a 1-L cylindrical flask purged with nitrogen, 40% glyoxal (299.98 g, 2.065 mol), a 40% formaldehyde aqueous solution (151.71 g, 2.20 mol), and acetic acid (154.30 g, 2.569 mol) were mixed. The mixture was heated at 50° C., and tert-butylamine (250.39 g, 3.422 mol) was added dropwise over 1 hour. The mixture after dropwise addition was stirred at 50° C. for 3 hours, and cooled to 25° C. The cooled reaction liquid was concentrated under reduced pressure at 60° C. for 1 hour. Dimethyl carbonate (313.03 g, 3.474 mol) was added to the concentrated residue, followed by stirring under reflux for 24 hours. The stirred reaction liquid was cooled to 25° C. and then concentrated under reduced pressure at 60° C. for 1 hour. Water (400 g) was added to the concentrated residue, followed by stirring at 25° C. for 1 hour. The stirred reaction liquid was cooled to 25° C. and then concentrated under reduced pressure at 60° C. for 1 hour. Acetone (500 g) was added to the concentrated residue, followed by stirring at 25° C. for 1 hour, and then filtration was performed. The filtered residue was dried under reduced pressure at 60° C. for 1 hour to give 100 g (24.1%) of [tBuI] [C]. ¹H-NMR (CDCl₃) δ (ppm)=7.35 (s, 2H), 1.75 (s, 18H)

### Production Example 2: Synthesis of (2-methoxyethyl) diethylmethylammonium methyl carbonate

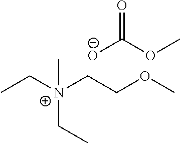

Methoxyethyldiethylamine (1100 g, 8.38 mol), dimethyl carbonate (1512.3 g, 16.79 mol), and methanol (274.8 g) were added to a 5-L autoclave purged with nitrogen. The mixture was stirred at 130° C. for 26 hours. 2732.5 g of the reaction liquid was obtained as a (2-methoxyethyl) diethylmethylammonium methyl carbonate methanol solution.

### Production Example 3: Synthesis of tri-n-butyl-n-hexylphosphonium hydrogen carbonate

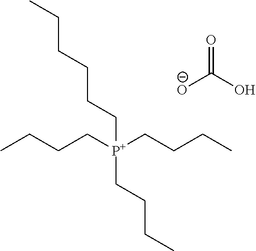

Tri-n-butyl-n-hexylphosphonium bromide (1.00 g, 2.72 mol) and potassium hydrogen carbonate (3.02 g, 30.1 mmol) were added to a 100-ml cylindrical flask purged with nitrogen. Further, dichloromethane (5 g) was added, followed by stirring at 25° C. for 10 hours. The obtained suspension was filtered at 25° C. The filtrate was concentrated under reduced pressure at 60′C to give 530 mg (yield: 55.9%) of tri-n-butyl-n-hexylphosphonium hydrogen carbonate. ¹H-NMR (CD₂Cl₃) δ (ppm)=2.36-2.26 (m, 8H), 1.49-1.35 (m, 16H), 1.24-1.20 (m, 4H), 0.86 (t, J=7.2 Hz, 9H), 0.79 (t, J=7.2 Hz, 3H)

### Production Example 4: Synthesis of N-hexadecylpyridinium hydrogen carbonate

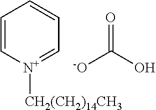

N-hexadecylpyridinium bromide (1.00 g, 2.60 mmol) and potassium hydrogen carbonate (3.00 g, 29.9 mmol) were added to a 100-ml cylindrical flask purged with nitrogen. Further, dichloromethane (5 g) was added, followed by stirring at 25° C. for 10 hours. The obtained suspension was filtered at 25° C. The filtrate was concentrated under reduced pressure at 60° C. to give 1.12 g (yield: quant.) of N-hexadecylpyridinium hydrogen carbonate.

¹H-NMR (CD₂Cl₃) δ (ppm)=9.54 (dd, J=1.2, 6.8 Hz, 2H), 8.54 (tt, J=1.2, 6.8 Hz, 1H), 8.13 (dd, 6.8, 7.6 Hz, 2H), 4.95 (t, J=7.6 Hz, 2H), 1.99 (t, J=7.6, 2H) 1.33-1.18 (m, 26H) 0.81 (t, J=6.8 Hz, 3H)

### Production Example 5: Synthesis of methyl-N-phenyl carbamate

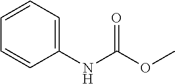

Methanol (25.7 g, 802 mmol) and triethylamine (100 mg, 0.98 mmol) were added to a 100-ml cylindrical flask purged with nitrogen. After heating to 50° C., phenyl isocyanate was added dropwise, followed by aging at 50° C. for 5 hours. The obtained reaction liquid was concentrated under reduced pressure at 60° C. to give 25.18 g (yield: 97.2%) of methyl-N-phenyl carbamate.

¹H-NMR (CD₂Cl₃) δ (ppm)=7.39 (d, J=7.6 Hz, 2H), 7.30 (t, J=8.4 Hz, 2H) 7.06 (tt, J=1.2, 7.2 Hz, 1H), 3.74 (s, 3H)

### Production Example 6: Synthesis of 2,2,2-trifluoroethyl-N-n-hexylcarbamate

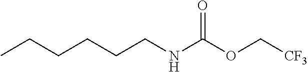

2,2,2-Trifluoroethanol (20.13 g, 200 mmol), toluene (20 g), and triethylamine (196 mg, 1.93 mmol) were added to a 100-ml cylindrical flask purged with nitrogen. After heating to 50° C., n-hexyl isocyanate was added dropwise, followed by aging at 50° C. for 5 hours. The obtained reaction liquid was concentrated under reduced pressure at 60° C. to give 24.70 g (yield: 86.9%) of 2,2,2-trifluoroethyl-N-n-hexylcarbamate.

¹H-NMR (CD₂Cl₁) δ (ppm)=4.46 (q, J=8.8 Hz, 2H), 4.46 (q, J=7.2 Hz, 2H), 1.50 (t, J=7.2 Hz, 2H), 1.35-1.29 (m, 6H), 0.89 (t, J=6.8 Hz, 3H)

### Production Example 7: Method for Producing methyl-N-2-ethylhexyl carbamate [MN2EHC]

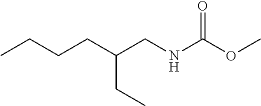

2-Ethylhexylamine (produced by Tokyo Chemical Industry Co., Ltd.) (50 g, 386 mmol) and potassium tert-butoxide (produced by Tokyo Chemical Industry Co., Ltd.) (433 mg, 3.86 mmol) were added and dissolved in dimethyl carbonate (produced by Tokyo Chemical Industry Co., Ltd.) (38.1 g, 424 mmol) in a 200-ml cylindrical flask purged with nitrogen. The obtained solution was stirred at 60° C. for 6 hours for reaction. The obtained reaction liquid was concentrated under reduced pressure at 60° C. for 1 hour to give 53.0 g (yield: 73.4%) of [MN22EHC] represented by the above formula. The ° H-NMR analysis results of the compound represented by the above formula are shown below.

¹H-NMR (CD₂Cl₂) δ (ppm)=3.62 (s, 3H), 3.06 (t, J=6.0 Hz, 2H), 1.40-1.23 (m, 9H), 0.92-0.85 (m, 6H)

### Production Example 8: Method for Producing toluene dimethylcarbamate ([TDMC])

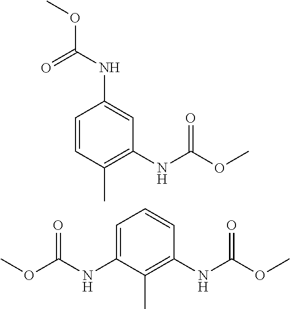

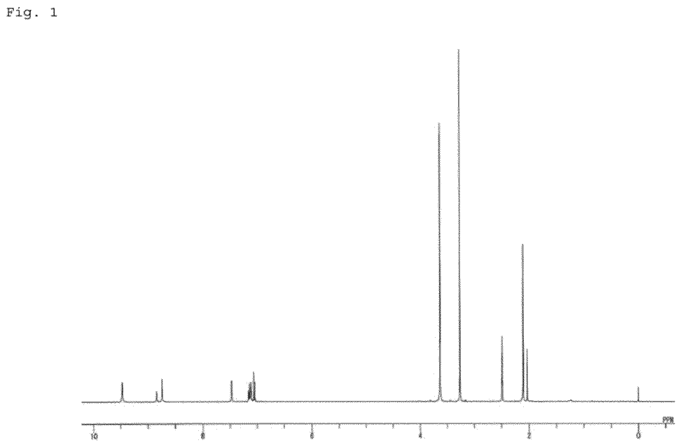

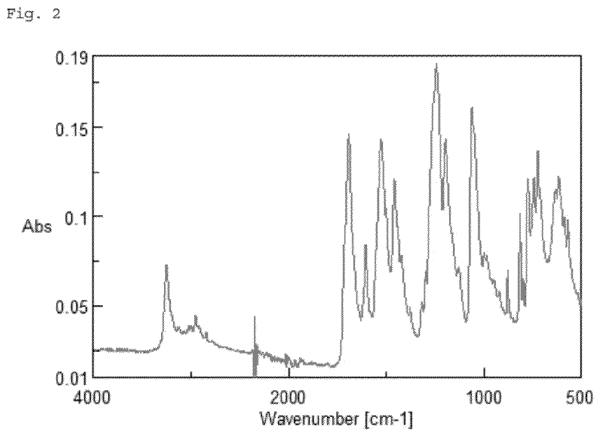

Methanol (45.7 g) and triethylamine (produced by Tokyo Chemical Industry Co., Ltd.) (5.80 mg, 0.574 mmol) were added to a 180-ml cylindrical flask purged with nitrogen. After heating to 60° C., toluene diisocyanate (produced by Tokyo Chemical Industry Co., Ltd.) (10.0 g, 57.4 mmol) was added dropwise at 60 to 65° C. The obtained solution was stirred at 60° C. for 2 hours for reaction. The obtained reaction liquid was concentrated under reduced pressure at 60° C. for 1 hour to give 13.1 g (yield: quant.) of [TDMC] represented by the above formula. This mixture is assumed to be a mixture of the compounds represented by the above formulas. FIG. 1 shows the ¹H-NMR (DMSO-d₆) analysis results of (TDMC), and FIG. 2 shows the IR analysis results.

### Production Example 9: Method for Producing triethylmethylanmonium methyl carbonate ([TEMA] [MC])

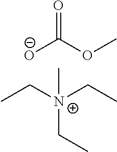

Triethylamine (produced by Tokyo Chemical Industry Co., Ltd.) (100 g, 988 mmol) and dimethyl carbonate (produced by Tokyo Chemical Industry Co., Ltd.) (150 g, 1.66 mol) were added to a 180-ml autoclave purged with nitrogen. The mixture was stirred at 125° C. for 12 hours and then cooled to 25° C. The cooled reaction liquid was concentrated under reduced pressure at 60° C. for 1 hour to give 121 g (yield: 64%) of [TEMA] [MC] represented by the above formula. The ¹H-NMR analysis results of the compound represented by the above formula are shown below.

¹H-NMR (CD₂Cl₂) δ (ppm)=3.44 (q, J=6.8, 6H), 3.37 (s, 3H), 1.32 (t, J=6.80, 9H)

### Production Example A-1: Synthesis of 1,3-di-tert-butylimidazolium methyl-N-phenyl carbamate ([DtBI] [MNPhC])

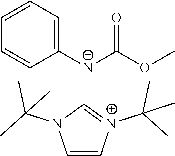

The di-tert-butylimidazolium hydrogen carbonate (7.28 g, 30.45 mmol) obtained in Production Example 1 and the methyl-N-phenyl carbamate (4.619 g, 30.56 mmol) obtained in Production Example 5 were added to a 200-ml cylindrical flask purged with nitrogen, and toluene (10 g) was added for suspension. The obtained suspension was stirred at 25° C. for 1 hour for reaction. The obtained reaction liquid was concentrated under reduced pressure at 60° C. for 1 hour to give 10.62 g (yield: quant.) of [DtBI] [MNPhC] represented by the above formula. The ¹H-NMR analysis results of the compound represented by the above formula are shown below.

¹H-NMR (MeOD-d₆) δ (ppm)=7.87 (s, 2H), 7.41 (d, J=8.0 Hz, 2H), 7.26 (t, J=8.4 Hz, 2H), 7.00 (t, J=7.2 Hz, 1H), 3.73 (s, 3H), 1.69 (s, 18H)

### Production Example A-2: Synthesis of 1,3-di-tert-butylimidazolium 2,2,2-trifluoroethyl-N-n-hexylcarbamate ([DtBI] [TFENPhC])

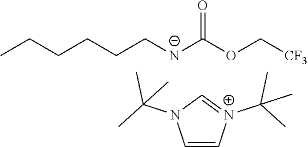

The di-tert-butylimidazolium hydrogen carbonate (1.00 g, 4.40 mmol) obtained in Production Example 1 and the 2,2,2-trifluoroethyl-N-n-hexylcarbamate (1.06 g, 4.40 mmol) obtained in Production Example 6 were added to a 100-ml cylindrical flask purged with nitrogen, and toluene (10 g) was added for suspension. The obtained suspension was stirred at 25° C. for 1 hour for reaction. The obtained reaction liquid was concentrated under reduced pressure at 60° C. for 1 hour to give 1.95 g (yield: quant.) of [DtBI] [TFENPhC] represented by the above formula. The ¹H-NMR analysis results of the compound represented by the above formula are shown below.

¹H-NMR (CD₂Cl₂) δ (ppm)=10.35 (a, 1H), 7.56 (s, 2H), 3.81 (q, J=9.6 Hz, 2H), 2.98 (q, J=6.4 Hz, 2H), 1.67 (9, 18H), 1.38-1.35 (m, 2H), 1.28-1.24 (m, 6H), 0.84 (t, J=6.0 Hz, 3H)

### Production Example A-3: Synthesis of 1,3-di-tert-butylimidazolium N-phenylacetamidate ([DtBI] [AcA])

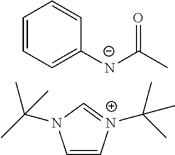

The di-tert-butylimidazolium hydrogen carbonate (5.00 g, 20.63 mmol) obtained in Production Example 1 and acetanilide (3.11 g, 23.03 mmol) were added to a 100-ml cylindrical flask purged with nitrogen, and toluene (15 g) was added for suspension. The obtained suspension was stirred at 25° C. for 6 hours for reaction. The obtained reaction liquid was concentrated under reduced pressure at 60° C. for 1 hour to give 7.69 g (yield: quant.) of [DtBI] [AcA] represented by the above formula. The ¹H-NMR analysis results of the compound represented by the above formula are shown below.

¹H-NMR (CD₂Cl₂) δ (ppm)=10.07 (br, 1H), 7.72 (dd, J=8.8, 1.2 Hz, 2H), 7.51 (s, 2H), 7.16 (dd, J=8.4, 7.6 Hz, 2H), 6.94 (tt, J=7.2, 1.2 Hz, 1H), 2.11 (s, 3H), 1.69 (s, 18H)

### Production Example A-4: Synthesis of 1,3-di-tert-butylimidazolium N,N′-dimethylurate ([DtBI] [DMU])

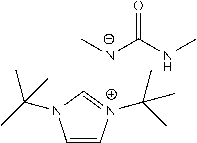

The di-tert-butylimidazolium hydrogen carbonate (5.00 g, 20.63 mmol) obtained in Production Example 1 and dimethylurea (1.81 g, 20.63 mmol) were added to a 100-ml cylindrical flask purged with nitrogen, and toluene (15 g) was added for suspension. The obtained suspension was stirred at 25° C. for 6 hours for reaction. The obtained reaction liquid was concentrated under reduced pressure at 60′C for 1 hour to give 6.68 g (yield: quant.) of [DtBI] [DMU] represented by the above formula. The ¹H-NMR analysis results of the compound represented by the above formula are shown below.

¹H-NMR (CD₂Cl₂) δ (ppm)=9.98 (br, 1H), 7.60 (s, 2H), 6.90 (br, 11), 2.58 (s, 3H), 2.57 (s, 3H), 1.66 (s, 18H) (0429)

### Production Example A-5: Synthesis of (2-methoxyethyl) diethylmethylammonium methyl-N-phenyl carbamate ([DEME] [MNPhC])

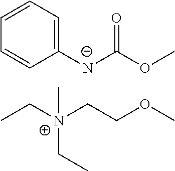

The (2-methoxyethyl)diethylmethylammonium methyl carbonate (5.90 g, 25.0 mmol) obtained in Production Example 2 and the methyl-N-phenyl carbamate (3.77 g, 25.0 mmol) obtained in Production Example 5 were added to a 50-ml eggplant flask purged with nitrogen, and tetrahydrofuran (10 g) was added for suspension. The obtained suspension was stirred at 60° C. for 3 hours for reaction. The obtained reaction liquid was cooled to 25° C., and then concentrated under reduced pressure at 60° C. for 1 hour to give 6.98 g (yield: 89.9%) of [DEME] [MNPhC] represented by the above formula. The ¹H-NMR analysis results of the compound represented by the above formula are shown below.

¹H-NMR (CDCl₃) δ (ppm)=7.46 (d, J=7.6 Hz, 2H), 7.20 (t, J=7.6 Hz, 2H), 6.94 (t, J=7.6 Hz, 1H), 3.68-3.66 (m, 5H), 3.49-3.43 (m, 6H), 3.25 (s, 3H), 3.10 (s, 3H), 1.23 (t, J=7.2 Hz, 6H)

### Production Example A-6: Synthesis of tri-n-butyl-n-hexylphosphonium methyl-N-phenyl carbamate ([TBHP] [MNPhC])

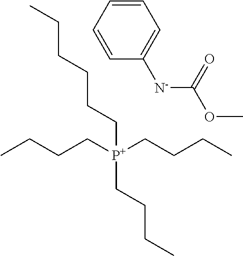

The tri-n-butyl-n-hexylphosphonium hydrogen carbonate (3.00 g, 8.60 mmol) obtained in Production Example 3 and the methyl-N-phenyl carbamate (1.36 g, 9.03 mmol) obtained in Production Example 5 were added to a 100-ml cylindrical flask purged with nitrogen, and dichloromethane (9 g) was added for suspension. The obtained suspension was stirred at 25° C. for 6 hours. After the stirred suspension was filtered, the filtered residue was dried under reduced pressure at 60° C. for 1 hour to give 3.44 g (yield: 91.4%) of [TBHP] [MNPhC] represented by the above formula. The ¹H-NMR analysis results of the compound represented by the above formula are shown below.

¹H-NMR (CD₂Cl₂) δ (ppm)=7.44 (d, J=8.0 Hz, 2H), 7.27 (t, J 7.2 Hz, 2H), 7.02 (tt, 7.6, 1.2 Hz, 1H), 3.76 (s, 3H), 2.39-2.30 (m, 8H), 1.56-1.45 (m, 16H), 1.33-1.29 (m, 4H), 0.96 (t, J=7.2 Hz, 9H), 0.89 (t, J=6.8 Hz, 3H)

### Production Example A-7: Synthesis of N-hexadecylpyridinium methyl-N-phenyl carbamate ([HdPy] [MNPhC])

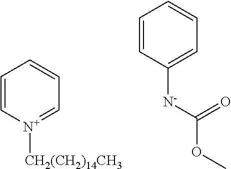

The N-hexadecylpyridinium hydrogen carbonate (3.00 g, 8.20 mmol) obtained in Production Example 4 and the methyl-N-phenyl carbamate (1.30 g, 8.61 mmol) obtained in Production Example 5 were added to a 100-ml cylindrical flask purged with nitrogen, and dichloromethane (9 g) was added for suspension. The obtained suspension was stirred at 25° C. for 6 hours. After the stirred suspension was filtered, the filtered residue was dried under reduced pressure at 60° C. for 1 hour to give 4.03 g (yield: quant.) of [HdPy] [MNPhC] represented by the above formula. The ¹H-NMR analysis results of the compound represented by the above formula are shown below.

¹H-NMR (CD₂Cl₂) δ (ppm)=9.41 (dd, J=7.2, 1.6 Hz, 2H), 8.48 (tt, J=8.0, 1.2 Hz, 1H), 8.09 (t, J=6.8 Hz, 2H), 7.45 (d, J=8.0 Hz, 2H), 7.28 (t, J=7.6 Hz, 2H), 7.03 (tt, J a 7.6, 1.2 Hz, 1H), 4.92 (t, J=7.6 Hz, 2H), 3.72 (s, 3H), 2.02 (m, 2H), 1.33-1.25 (m, 26H), 0.872 (t, J=7.2 Hz, 3H)

### Production Example A-8: Method for Producing trimethyl-n-octylammonium dimethylurea (([TMGA] [DMU])

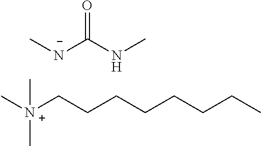

The trimethyl-n-octylammonium methyl carbonate (27.8 g, 113 mol) obtained in Comparative Production Example A-1 and dimethylurea (produced by Tokyo Chemical Industry Co., Ltd.) (10.0 g, 113 mmol) were added to a 200-ml cylindrical flask purged with nitrogen, and methanol (17.49 g) was added for dissolution. The obtained solution was stirred under reflux for 5 hours for reaction. The obtained reaction liquid was concentrated under reduced pressure at 60° C. for 1 hour to give 29.13 g (yield: quant.) of [TMOA] [DMU] represented by the above formula. The ¹H-NMR analysis results of the compound represented by the above formula are shown below.

¹H-NMR (CD₂Cl₂) δ (ppm)=3.38 (t, J=4.0 Hz, 2H), 3.25 (s, 9H), 2.63 (s, 6H), 1.74-1.70 (m, 2H), 1.35-1.28 (m, 10H), 0.88 (t, J=7.2 Hz, 3H)

### Production Example A-9: Method for Producing trimethyl-n-octylammonium methyl-N-2-ethylhexyl carbamate ([TMOA] [MN2EHC])

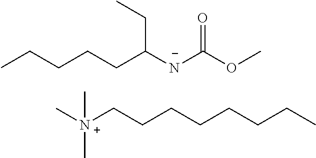

The trimethyl-n-octylammonium methyl carbonate (26.1 g, 106 mol) obtained in Comparative Production Example A-1 and [MN2EHC](20.0 g, 106 mmol) obtained in Production Example 7 were added to a 200-ml cylindrical flask purged with nitrogen, and methanol (92 g) was added for dissolution. The obtained solution was stirred under reflux for 6 hours for reaction. The obtained reaction liquid was concentrated under reduced pressure at 60° C. for 1 hour to give 42.15 g (yield: quant.) of [TMQA] [MN2EHC] represented by the above formula. The ¹H-NMR analysis results of the compound represented by the above formula are shown below. ¹H-NMR (CD₂Cl₂) δ (ppm)=3.60 (s, 3H), 3.43 (t, J=7.2 Hz, 2H), 3.30 (s, 9H), 3.06 (t, J=4.0 Hz, 2H), 1.73-1.69 (m, 2H), 1.39-1.24 (m, 19H) 0.90-0.85 (m, 9H)

### Production Example A-10: Method for Producing trimethyl-n-octylammonium toluene dimethylcarbamate ([TMOA] [TDMC])

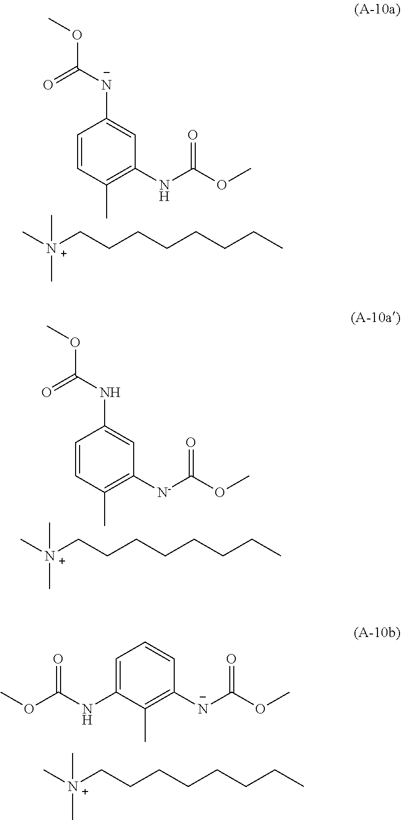

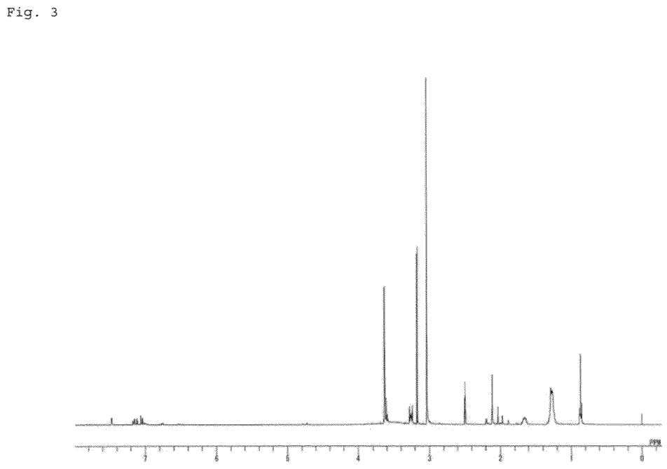

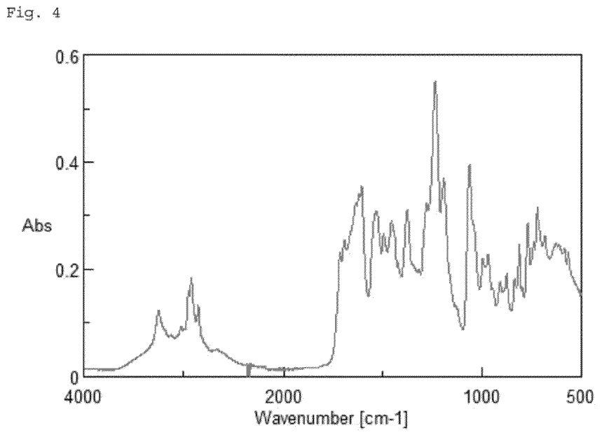

The trimethyl-n-octylammonium methyl carbonate (5.14 g, 20.9 mmol) obtained in Comparative Production Example A-1 and the toluene bis(methylcarbamate) (5.00 g, 20.9 mmol) obtained in Production Example 8 were added to a 200-ml cylindrical flask purged with nitrogen, and THF (15 g) was added for dissolution. The obtained solution was stirred under reflux for 3 hours for reaction. The obtained reaction liquid was concentrated under reduced pressure at 60° C. for 1 hour to give 9.94 g (yield: quant.) of a mixture of compounds represented by the above formulas ([TMOA] [TDMC]). FIG. 3 shows the H-NMR (DMSO-d₆) analysis results of the obtained mixture [TMOA] [TDMC], and FIG. 4 shows the IR analysis results.

### Production Example A-11: Method for Producing trimethyl-n-octylammonium methyl-N-phenyl carbamate ([TMOA] [MNPhC])

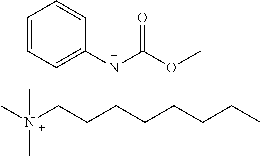

The trimethyl-n-octylammonium methyl carbonate (3 g, 12.1 mol) obtained in Comparative Production Example A-1 and the methyl-N-phenyl carbamate (1.82 g, 12.1 mmol) obtained in Production Example 5 were added to a 200-ml cylindrical flask purged with nitrogen, and THF (5 g) was added for dissolution. The obtained solution was stirred under reflux for 4 hours for reaction. The obtained reaction liquid was concentrated under reduced pressure at 60° C. for 1 hour to give 4.23 g (yield: quant.) of [TMOA] [MNPhC] represented by the above formula. The ¹H-NMR analysis results of the compound represented by the above formula are shown below.

¹H-NMR (DMSO-d₆) δ (ppm)=7.45 (dd, J=8.4, 0.8 Hz, 2H), 7.26 (t, J=7.6 Hz, 2H), 6.97 (tt, J=7.2, 0.8 Hz, 1H), 3.65 (s, 3H), 3.37 (t, J=4.0 Hz, 2H), 3.04 (s, 9H), 16.7-1.643 (m, 2H), 1.29-1.28 (m, 10H), 0.87 (t, J=7.2 Hz, 3H)

### Production Example A-12: Method for Producing trimethyl-n-octylammonium methyl-N-phenyl carbamate ([TMOA] [MNPhC])

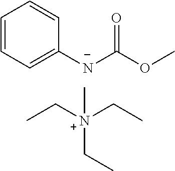

[TMOA] [MC](10 g, 52.2 mmol) obtained in Production Example 9 and the methyl-N-phenyl carbamate (7.89 g, 52.2 mmol) obtained in Production Example 5 were added to a 100-ml autoclave purged with nitrogen, and THE (20 g) was added for dissolution. The mixture was stirred under reflux conditions for 3 hours and then cooled to 25° C. The cooled reaction liquid was concentrated under reduced pressure at 60° C. for 1 hour to give 16.3 g (yield: quant.) of [TEMA] [MNPhC] represented by the above formula. The ¹H-NMR analysis results of the compound represented by the above formula are shown below.

¹H-NMR (DMSO-d₆) δ (ppm)=¹H-NMR (DMSO-d₆) δ (ppm)=7.42 (dd, J=8.8, 1.2 Hz, 2H), 7.18 (t, J=7.6 Hz, 2H), 6.85 (tt, J=7.6, 1.2 Hz, 1H), 3.58 (s, 3H), 3.24 (q, J=7.6 Hz, 6H), 2.88 (s, 3H), 1.18 (t, J=7.2 Hz, 9H)

### Comparative Production Example A-1: Method for Producing trimethyl-n-octylammonium methyl carbonate ([TMOA] [MC])

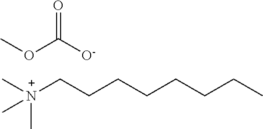

Dimethyl-n-octylamine (produced by Tokyo Chemical Industry Co., Ltd.) (10.00 g, 63.5 mmol) and dimethyl carbonate (17.1 g, 190 mmol) were added to a 100-ml autoclave purged with nitrogen. The mixture was stirred at 120′C for 12 hours and then cooled to 25° C. The cooled reaction liquid was concentrated under reduced pressure at 60° C. for 1 hour to give 1.6.2 g (yield: quant.) of [TMOA] [MC] represented by the above formula. The ¹H-NMR analysis results of the compound represented by the above formula are shown below.

¹H-NMR (DMSO-d₄) δ (ppm)=3.27 (t, J=8.4 Hz, 2H), 3.16 (s, 3H), 3.04 (br, 9H), 1.66 (br, 2H) 1.27 (br, 10H), 0.87 (t, J=6.8 Hz, 3H)

### Production Example B-1: Synthesis of 2,2,2-trifluoroethanol-blocked HDI biuret

150.0 g (NCO group: 0.81 mol) of HDI biuret (Desmodur N3200A, NCO group content: 22.8(%), produced by Sumika Covestro Urethane Co., Ltd.) and 73.3 g of methyl isobutyl ketone (“MIBK” below) were placed in a 200-mL three-necked reactor purged with nitrogen and heated to 65° C., followed by addition of 1.4 g of triethylamine (“TEA” below). Thereafter, 27.0 g (0.99 mol) of 2,2,2-trifluoroethanol (“TFE” below) and 79.4 g of MIBK were added dropwise to the reactor and stirred at 65° C. for 2 hours. Then, the disappearance of the infrared absorption peak of isocyanate group near 2270 cm⁻¹ was confirmed by infrared spectroscopic analysis. The obtained reaction solution was concentrated under reduced pressure to remove TEA and most of MIBK, and 59.9 g of MIBK was added, thus obtaining 306.1 g of a MIBK solution of TFE-blocked HDI biuret. The obtained TFE-blocked HDI biuret had a solids content of 76.1% and an effective NCO group content of 11.2%.

### Example 1

The TFE-blocked HDI biuret obtained in Production Example B-1, a polyester polyol (P-510, produced by Kuraray Co., Ltd.), and the blocking agent dissociation catalyst [DtBI] [MNPhC]produced in Production Example A-1 were added such that the formulation of a thermosetting resin composition satisfied that effective NCO group (mol):hydroxyl group (mol):curing catalyst (mol)=1.00:0.95:0.05, The mixture was then stirred for 30 minutes, thus preparing a thermosetting resin composition.

About 0.6 mL of the prepared thermosetting resin composition was poured onto the hot plate of the automatic curing time measuring device that had been heated to 80° C. or 100° C. in advance, and stirring was performed. During this procedure, the curing time was measured and evaluated, taking the time between the stirring torque immediately after the start of stirring of less than 1% (0.04 mN-m) and the stirring torque exceeding 50% (0.86 mN-m) as the curing time. Table 1 shows the results.

### Examples 2 to 11 and Comparative Examples 1 and 2

Thermosetting resin compositions were prepared in the same manner as in Example 1, except that the blocking agent dissociation catalysts shown in Examples A-2 to A-11, or Comparative Production Example A-1 and Production Example 1 were used. The curing time was measured and evaluated. Table 5 shows the results.

TABLE 5

| | | Heating temperature (° C.) | |
|---|---|---|---|
| | Blocking agent | 80° C. | 100° C. |
| | dissociation catalyst | Curing time (min) | |
| Example 1 | [DtBl][MNPhC] | 30 | 9 |
| Example 2 | [DtBl][TFENPhC] | 30 | 11 |
| Example 3 | [DtBl][AcA] | 28 | 11 |
| Example 4 | [DtBl][DMU] | 25 | 14 |
| Example 5 | [DEME][MNPhC] | 30 | 8 |
| Example 6 | [TMOA][DMU] | 40 | 14 |
| Example 7 | [TMOA][MN2EHC] | 38 | 12 |
| Example 8 | [TMOA][MNPhC] | 42 | 8 |
| Example 9 | [TEMA][MNPhC] | 45 | 13 |
| Example 10 | tBtlPhU | 36 | 10 |
| Example 11 | [TMOA][TDMC] | 42 | 15 |
| Comparative | [TMOA][MC] | Not cured even | 20 |
| Example 1 | | after 60 minutes | |
| Comparative | [tBul][C] | Not cured even | 30 |
| Example 2 | | after 60 minutes | |

1. A blocking agent dissociation catalyst for blocked isocyanates comprising an onium salt represented by the following formula (1): 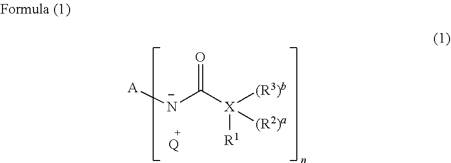
wherein
n represents an integer of 1 or more,
R¹, R², and R³ are the same or different, and each represents a hydrogen atom or a substituted or unsubstituted hydrocarbon group,
A represents a hydrogen atom or a substituted or unsubstituted hydrocarbon group when n=1, and represents a substituted or unsubstituted n-valent hydrocarbon group when n represents an integer of 2 or more,
wherein some or all of R¹, R², and R³ may be bonded together to form one or more ring structures,
X represents a carbon atom, a nitrogen atom, or an oxygen atom,
a represents 0 or 1,
b represents 0 or 1,
wherein when X represents a carbon atom, a=1 and b=1, when X represents a nitrogen atom, a=1 and b=0, and when X represents an oxygen atom, a=0 and b=0, and
Q⁺ represents a cation.

2. The blocking agent dissociation catalyst for blocked isocyanates according to claim 1, wherein in formula (1), Q⁺ represents an organic cation.

3. The blocking agent dissociation catalyst for blocked isocyanates according to claim 1,
wherein
in formula (1), Q⁺ represents an organic cation represented by the following formula (2) or formula (3): 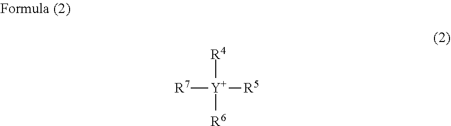
wherein
R⁴, R⁵, R⁶, and R⁷ are the same or different, and each represents a substituted or unsubstituted hydrocarbon group, wherein some or all of R⁴, R, R⁶, and R⁷ may be bonded together to form one or more ring structures, and
Y⁺ represents a nitrogen cation or a phosphorus cation; 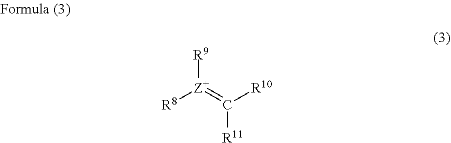
wherein
R⁸, R⁹, and R¹¹ are the same or different, and each represents a substituted or unsubstituted hydrocarbon group,
R¹⁰ represents a hydrogen atom, or a substituted or unsubstituted hydrocarbon group, wherein some or all of R⁸, R⁹, R¹⁰, and R¹¹ may be bonded together to form one or more ring structures, and
Z⁺ represents a nitrogen cation or a phosphorus cation.

4. The blocking agent dissociation catalyst for blocked isocyanates according to claim 3,
wherein
Y⁺ represents a nitrogen cation, or
the organic cation represented by formula (3) represents an organic cation represented by formula (3-1) or formula (3-2): 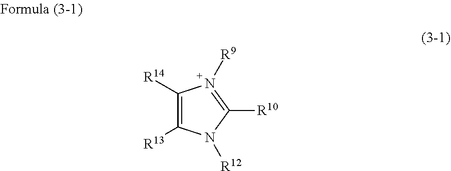
wherein
R⁹ and R¹² each represent a substituted or unsubstituted hydrocarbon group, and
R¹⁰, R¹³, and R¹⁴ are the same or different, and each represents a hydrogen atom or a substituted or unsubstituted hydrocarbon group, wherein some or all of R⁹, R¹⁰, R¹², R¹³, and R¹⁴ may be bonded together to form one or more ring structures; 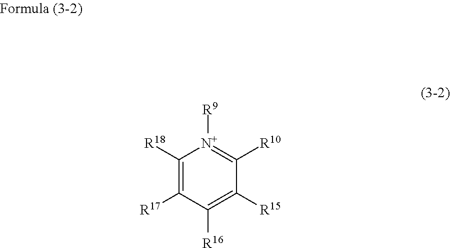
wherein
R⁹ represents a substituted or unsubstituted hydrocarbon group, and
R¹⁰, R¹⁵, R¹⁶, R¹⁷, and R¹⁸ are the same or different, and each represents a hydrogen atom or a substituted or unsubstituted hydrocarbon group, wherein some or all of R⁹, R¹⁰, R¹⁵, R¹⁶, R¹⁷, and
R¹⁸ may be bonded together to form one or more ring structures.

5. The blocking agent dissociation catalyst for blocked isocyanates according to claim 1,
wherein
when n=1, A represents a hydrogen atom, a substituted or unsubstituted aliphatic hydrocarbon group, a substituted or unsubstituted alicyclic hydrocarbon group, a substituted or unsubstituted aromatic hydrocarbon group, or a substituted or unsubstituted aromatic aliphatic hydrocarbon group, and
when n represents an integer of 2 or more, A represents a substituted or unsubstituted n-valent aliphatic hydrocarbon group, a substituted or unsubstituted n-valent alicyclic hydrocarbon group, a substituted or unsubstituted n-valent aromatic hydrocarbon group, or a substituted or unsubstituted n-valent aromatic aliphatic hydrocarbon group.

6. The blocking agent dissociation catalyst for blocked isocyanates according to claim 1, wherein n represents an integer of 1 to 20.

7. The blocking agent dissociation catalyst for blocked isocyanates according to claim 1,
wherein
when X represents a carbon atom, R¹, R², and R³ are the same or different, and each represents a hydrogen atom or a substituted or unsubstituted alkyl group,
when X represents a nitrogen atom, R¹ and R² are the same or different, and each represents a hydrogen atom or a substituted or unsubstituted alkyl group, and
when X represents an oxygen atom, R¹ represents a substituted or unsubstituted alkyl group.

8. A blocked isocyanate composition comprising the blocking agent dissociation catalyst for blocked isocyanates of claim 1 and a blocked isocyanate compound.

9. The blocked isocyanate composition according to claim 8, wherein the blocked isocyanate compound is a blocked isocyanate compound blocked with at least one blocking agent selected from the group consisting of alcohol compounds, phenol compounds, amine compounds, lactam compounds, oxime compounds, keto-enol compounds, active methylene compounds, pyrazole compounds, triazole compounds, imide compounds, mercaptan compounds, imine compounds, urea compounds, and diaryl compounds.

10. The blocked isocyanate composition according to claim 8, wherein the blocked isocyanate compound is a blocked isocyanate compound blocked with a fluorinated alcohol compound.

11. A thermosetting resin composition comprising the blocked isocyanate composition of claim 8 and a compound having an isocyanate-reactive group.

12. The thermosetting resin composition according to claim 11, wherein the compound having an isocyanate-reactive group is a polyol compound.

13. A cured product obtained by curing the thermosetting resin composition of claim 11.

14. A method for producing a cured product, comprising curing the thermosetting resin composition of claim 11 by heating.

15. An onium salt represented by the following formula (1): 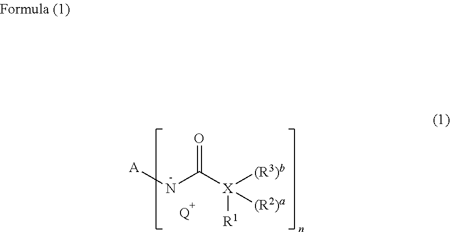
wherein
n represents an integer of 1 or more,
R¹, R², and R³ are the same or different, and each represents a hydrogen atom or a substituted or unsubstituted hydrocarbon group,
A represents a hydrogen atom or a substituted or unsubstituted hydrocarbon group when n=1, and represents a substituted or unsubstituted n-valent hydrocarbon group when n represents an integer of 2 or more,
wherein some or all of R¹, R², and R³ may be bonded together to form one or more ring structures,
X represents a carbon atom, a nitrogen atom, or an oxygen atom,
a represents 0 or 1,
b represents 0 or 1,
wherein when X represents a carbon atom, a=1 and b=1, when X represents a nitrogen atom, a=1 and b=0, and when X represents an oxygen atom, a=0 and b=0, and
Q⁺ represents an organic cation represented by the following formula (2) or formula (3): 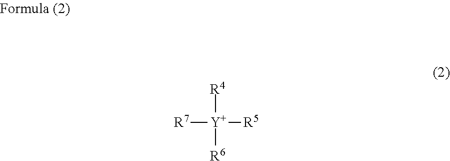
wherein
R⁴, R⁵, R⁶, and R⁷ are the same or different, and each represents a substituted or unsubstituted hydrocarbon group, wherein some or all of R⁴, R⁵, R⁶, and R⁷ may be bonded together to form one or more ring structures, and
Y⁺ represents a nitrogen cation or a phosphorus cation; 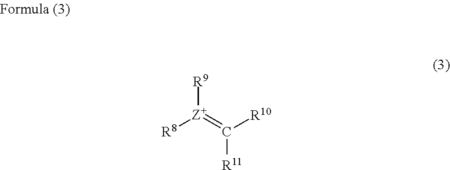
wherein
R⁸, R⁹, and R¹¹ are the same or different, and each represents a substituted or unsubstituted hydrocarbon group,
R¹⁰ represents a hydrogen atom or a substituted or unsubstituted hydrocarbon group, wherein some or all of R⁸, R⁹, R¹⁰, and R¹¹ may be bonded together to form one or more ring structures, and
Z⁺ represents a nitrogen cation or a phosphorus cation;
with the proviso that 1,3-di-isopropyl-4,5-dimethylimidazolium N,N′-diphenylureate is excluded.

16. The onium salt according to claim 15,
wherein
Y⁺ represents a nitrogen cation, or
the organic cation represented by formula (3) is an organic cation represented by formula (3-1) or formula (3-2): 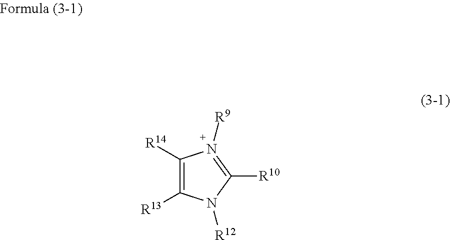
wherein
R⁹ and R¹² each represent a substituted or unsubstituted hydrocarbon group, and
R¹⁰, R¹³, and R¹⁴ are the same or different, and each represents a hydrogen atom or a substituted or unsubstituted hydrocarbon group, wherein some or all of R⁹, R¹⁰, R¹², R¹³, and R¹⁴ may be bonded together to form one or more ring structures; 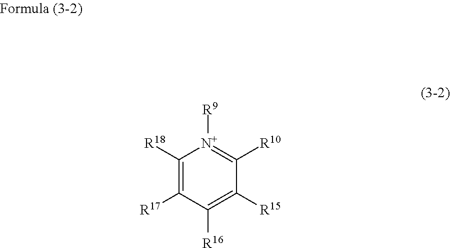
wherein
R⁹ represents a substituted or unsubstituted hydrocarbon group, and
R¹⁰, R¹⁵, R¹⁶, R¹⁷, and R¹⁸ are the same or different, and each represents a hydrogen atom or a substituted or unsubstituted hydrocarbon group, wherein some or all of R⁹, R¹⁰, R¹⁵, R¹⁶, R¹⁷, and
R¹⁸ may be bonded together to form one or more ring structures.

17. The onium salt according to claim 16,
wherein
when n=1, A represents a hydrogen atom, a substituted or unsubstituted aliphatic hydrocarbon group, a substituted or unsubstituted alicyclic hydrocarbon group, a substituted or unsubstituted aromatic hydrocarbon group, or a substituted or unsubstituted aromatic aliphatic hydrocarbon group, and
when n represents an integer of 2 or more, A represents a substituted or unsubstituted n-valent aliphatic hydrocarbon group, a substituted or unsubstituted n-valent alicyclic hydrocarbon group, a substituted or unsubstituted n-valent aromatic hydrocarbon group, or a substituted or unsubstituted n-valent aromatic aliphatic hydrocarbon group.

18. The onium salt according to any claim 15,
wherein n represents an integer of 1 to 20.

19. The onium salt according to claim 15, containing one anion selected from (4-1) to (4-4) and one organic cation selected from (b-i) and (b-ii): 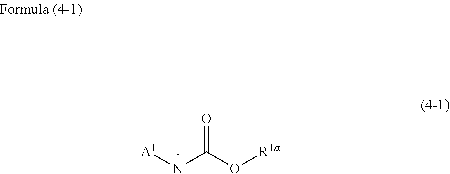
wherein
A¹ represents a substituted or unsubstituted aromatic hydrocarbon group, and
R^{1a} represents a substituted or unsubstituted aliphatic hydrocarbon group; 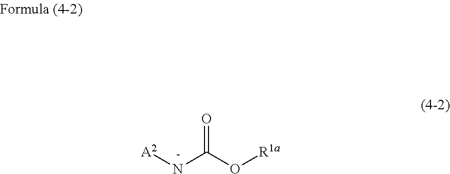
wherein
A² and R^{1a} are the same or different, and each represents a substituted or unsubstituted aliphatic hydrocarbon group; 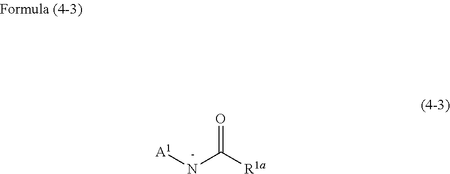
wherein A² and R^{1a} are as defined above; 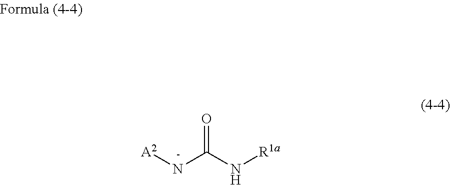
wherein A² and R^{1a} are as defined above;
(b-i) an organic cation represented by formula (2): 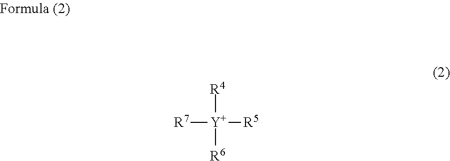
wherein
R⁴, R⁵, R⁶, and R⁷ are the same or different, and each represents a substituted or unsubstituted hydrocarbon group, wherein some or all of R⁴, R⁵, R⁶, and R⁷ may be bonded together to form one or more ring structures, and
Y⁺ represents a nitrogen cation;
(b-ii) an organic cation represented by the following formula (3-1): 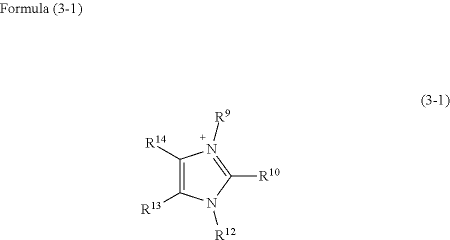
wherein
R⁹ and R¹² each represent a substituted or unsubstituted hydrocarbon group, and
R¹⁰, R¹³, and R¹⁴ are the same or different, and each represents a hydrogen atom or a substituted or unsubstituted hydrocarbon group, wherein some or all of R⁹, R¹⁰, R¹², R¹³, and R¹⁴ may be bonded together to form one or more ring structures.

20. The onium salt according to claim 15, containing one anion selected from (a-i) to (a-vi) and one organic cation selected from (b-i) and (b-ii): 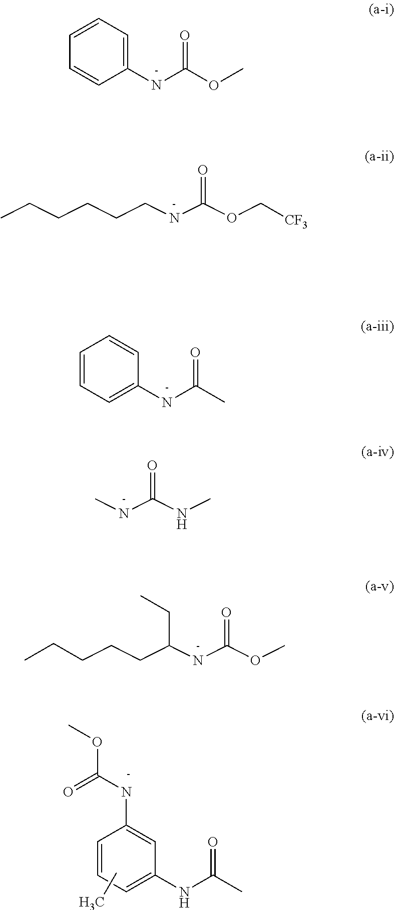
(b-i) an organic cation represented by formula (2): 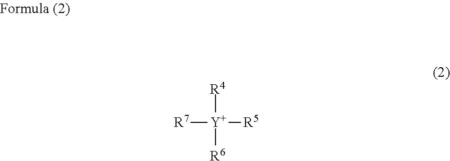
wherein
R⁴, R⁵, R⁶, and R⁷ are the same or different, and each represents a substituted or unsubstituted hydrocarbon group, wherein some or all of R⁴, R, R⁶, and R⁷ may be bonded together to form one or more ring structures, and
Y⁺ represents a nitrogen cation;
(b-ii) an organic cation represented by the following formula (3-1): 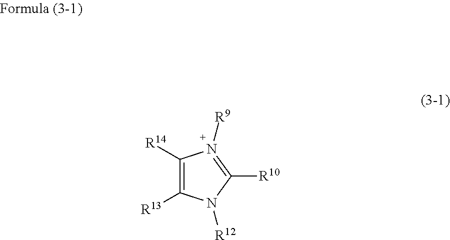
wherein
R⁹ and R¹² each represent a substituted or unsubstituted hydrocarbon group, and
R¹⁰, R¹³, and R¹⁴ are the same or different, and each represents a hydrogen atom or a substituted or unsubstituted hydrocarbon group, wherein some or all of R⁹, R¹⁰, R¹², R¹³, and R¹⁴ may be bonded together to form one or more ring structures.

21. The onium salt according to claim 15, wherein the onium salt is represented by any one of the following: 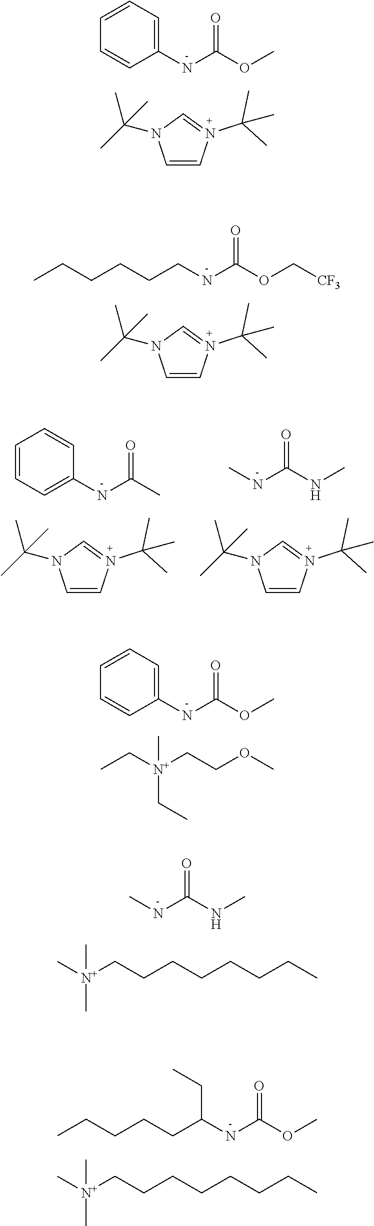 [chemical structure]
